(19) United States
(12) Patent Application Publication
(10) Pub. No.: US 2006/0252916 A1
(43) Pub. Date: Nov. 9, 2006

(54) Modified glucagon-like peptide-1 analogs

(75) Inventors: Richard Dennis DiMarchi, Greenfield, IN (US); Lianshan Zhang, Carmel (IN)

Correspondence Address: ELI LILLY & COMPANY, PATENT DIVISION, P.O. BOX 6288, INDIANAPOLIS, IN, 46206-6288, US

(73) Assignee: ELI LILLY AND COMPANY, INDIANAPOLIS, IN (US)

(21) Appl. No.: 10/516,490

(22) PCT Filed: Jun. 2, 2003
(86) PCT No.: PCT/US03/15395

§ 371 Date: Dec. 1, 2004

Related U.S. Application Data
(60) Provisional application No. 60/385,927, filed on Jun. 4, 2002.

(51) Int. Cl. A61K 38/26; C07K 14/605

(52) U.S. Cl. 530/324; 514/12

(57) ABSTRACT

The invention encompasses GLP-1 compounds containing a GLP-1 peptide or a GLP-1 peptide with an extended C-terminus that is modified with a reactive group that is capable of forming covalent bonds with a blood component to form a conjugate. The conjugates may be formed in vivo or ex vivo. Methods of treating a subject in need of GLP-1 receptor stimulation using these GLP-1 compounds are also disclosed.

### BACKGROUND OF THE INVENTION

A large body of pre-clinical and clinical research data suggests that glucagon-like pepide-1 (GLP-1) shows great promise as a treatment for non-insulin dependent diabetes mellitus (NIDDM) especially when oral agents begin to fail. GLP-1 induces numerous biological effects such as stimulating insulin secretion, inhibiting glucagon secretion, inhibiting gastric emptying, enhancing glucose utilization, and inducing weight loss. Further, pre-clinical studies suggest that GLP-1 may also act to prevent the pancreatic β cell deterioration that occurs as the disease progresses. Perhaps the most salient characteristic of GLP-1 is its ability to stimulate insulin secretion without the associated risk of hypoglycemia that is seen when using insulin therapy or some types of oral therapies that act by increasing insulin expression.

As NIDDM progresses, it becomes extremely important to achieve near normal glycemic control and thereby minimize the complications associated with prolonged hyperglycemia. GLP-1 would appear to be the drug of choice. However, the usefulness of therapy involving GLP-1 peptides has been limited by the fact that GLP-1(1-37) is poorly active, and the two naturally occurring truncated peptides, GLP-1(7-37)OH and GLP-1(7-36)NH₂, are rapidly cleared in vivo and have extremely short in vivo half-lives.

It is known that endogenously produced dipeptidyl-peptidase IV (DPP-IV) inactivates circulating GLP-1 peptides by removing the N-terminal histidine and alanine residues and is a major reason for the short in vivo half-life. Thus, recent efforts have focused on the development of GLP-1 peptides that are resistant to DPP-IV degradation. Some of these resistant peptides have modifications at the N-terminus (See U.S. Pat. No. 5,705,483), and some are derivatized GLP-1 peptides wherein large acyl groups that prevent DPP-IV from accessing the N-terminus of the peptide are attached to various amino acids (See WO 98/08871); In an alternative approach, GLP-1 peptides that are resistant to degradation have been sought through modification of GLP-1 peptides with reactive groups capable of covalently bonding to blood components (See U.S. Pat. No. 6,329,336).

The present invention addresses the need for GLP-1 peptides that are resistant to degradation through the development of novel GLP-1 compounds that contain GLP-1 peptides that are modified with reactive groups that interact with blood components to form conjugates. These conjugates increase the biological half-lives of the GLP-1 peptide and improve bio-availability. The increased stability of these novel GLP-1 peptides is achieved while maintaining their biological activity. Thus, the present invention makes possible therapy which involves delivering biologically active GLP-1 peptides such that therapeutic serum levels are achieved.

### SUMMARY OF THE INVENTION

It has now been found that GLP-1 peptides can be modified with reactive groups capable of forming covalent bonds to yield GLP-1 compounds, which can then be conjugated to blood components so as to stabilize the GLP-1 peptides.

One embodiment of the present invention is a GLP-1 compound having a GLP-1 peptide modified with an activated disulfide bond group or S-sulfonate, the GLP-1 peptide having the amino acid sequence of formula 1 (SEQ ID NO:1) provided that the GLP-1 compound does not have certain sequences as described herein.

Yet another embodiment of the present invention is a GLP-1 compound having a GLP-1 peptide modified with an activated disulfide bond group or S-sulfonate, the GLP-1 peptide having the amino acid sequence of formula 3 (SEQ ID NO:3) provided that if Xaa₃₉, Xaa₄₀, Xaa₄₁, Xaa₄₂, Xaa₄₃, Xaa₄₄, Xaa₄₅, Xaa₄₆, or Xaa₄₇ is absent each amino acid downstream is absent and further provided that the GLP-1 peptide does not have the following C-terminal amino acid extension beginning at Xaa₃₆: Gly-Pro-Ser-Ser-Gly-Ala-Pro-Pro-Pro-Ser-NH₂.

A further embodiment of the present invention is a GLP-1 compound having a GLP-1 peptide modified with an activated disulfide bond group or S-sulfonate, the GLP-1 peptide having the amino acid sequence of formula 5 (SEQ ID NO:5), wherein said GLP-1 peptide is modified at Lys³⁷, and provided that the GLP-1 compound does not have certain sequences as described herein.

Yet another embodiment of the present invention is a GLP-1 compound having a GLP-1 peptide modified with an activated disulfide bond group or S-sulfonate, the GLP-1 peptide having the amino acid sequence of formula 8 (SEQ ID NO:8), wherein said extended GLP-1 peptide is modified at a single Lys which occurs at one of Xaa₃₇, Xaa₃₈, Xaa₃₉, Xaa₄₀, Xaa₄₁, Xaa₄₂, Xaa₄₃, Xaa₄₄; Xaa₄₅, Xaa₄₆, Xaa₄₇, or Xaa₄₈; and provided that if Xaa₃₉, Xaa₄₀, Xaa₄₁, Xaa₄₂, Xaa₄₃, Xaa₄₄, Xaa₄₅, Xaa₄₆, or Xaa₄₇ is absent each amino acid downstream is absent and further provided that the GLP-1 peptide does not have the following C-terminal amino acid extension beginning at Xaa₃₆: Gly-Pro-Ser-Ser-Gly-Ala-Pro-Pro-Pro-Ser-NH₂.

Another embodiment of the present invention is a GLP-1 compound having a GLP-1 peptide modified with a succimimidyl group and a maleimido group, the GLP-1 peptide having the amino acid sequence of formula 9 (SEQ ID NO: 9), provided that the GLP-1 compound does not have certain sequences as described herein. Preferred embodiments of formulas 1 through 15 include GLP-1 peptides that have valine or glycine at position 8 and glutamic acid at position 22.

The present invention also encompasses a method of stimulating the GLP-1 receptor in a subject in need of such stimulation, said method comprising the step of administering to the subject an effective amount of the GLP-1 peptides described herein. Subjects in need of GLP-1 receptor stimulation include those with non-insulin dependent diabetes, stress-induced hyperglycemia, and obesity.

### DETAILED DESCRIPTION OF THE INVENTION

A GLP-1 compound of the present invention encompasses a GLP-1 peptide that has been modified by attaching a reactive group that is capable of covalently binding to a blood component.

A GLP-1 peptide is a polypeptide having sufficient similarity to GLP-1(7-37)OH such that the GLP-1 peptide exhibits insulinotrophic activity. Accordingly, GLP-1 peptides of the present invention include naturally occurring or native GLP-1 peptides. Preferably, the GLP-1 peptides of the present invention have various amino acid changes relative to the native GLP-1 molecules and have sufficient similarity to GLP-1(7-37)OH such that the GLP-1 peptides exhibits insulinotrophic activity. The various amino acid changes may occur through changes to the native GLP-1 molecules with naturally occurring or non-naturally occurring amino acids. The “extended GLP-1 peptides” according to the present invention have various amino acid substitutions relative to the native GLP-1(7-37) or GLP-1(7-36) molecule and have additional amino acids extending from the C-terminus.

Native GLP-1(7-37)OH has the amino acid sequence of SEQ ID NO:16: ⁷His-Ala-Glu-¹⁰Gly-Thr-Phe-Thr-Ser-¹⁵Asp-Val-Ser-Ser-Tyr-²⁰Leu-Glu-Gly-Gln-Ala-²⁵Ala-Lys-Glu-Phe-Ile-³⁰Ala-Trp-Leu-Val-Lys-³⁵Gly-Arg-³⁷Gly (SEQ ID NO:16).

The native GLP-1 molecule is also amidated in vivo such that the glycine residue at position 37 is replaced with an amide group. By custom in the art, the amino terminus of GLP-1(7-37)OH has been assigned residue number 7 and the carboxy-terminus, number 37. The other amino acids in the polypeptide are numbered consecutively, as shown in SEQ ID NO:16. For example, in SEQ ID NO:16, position 12 is phenylalanine and position 22 is glycine. The same numbering system is used for the GLP-1 peptides and extended GLP-1 peptides of the present invention.

GLP-1 peptides include “GLP-1 analogs” which have sufficient homology to GLP-1(7-37)OH, GLP-1(7-36)NH₂ or a fragment of GLP-1(7-37)OH or GLP-1(7-36)NH₂ such that the analog has insulinotropic activity. Preferably, a GLP-1 analog has the amino acid sequence of GLP-1(7-37)OH or a fragment thereof, modified so that from one, two, three, four, five, or six amino acids differ from the amino acid in the corresponding position of GLP-1(7-37)OH or a fragment of GLP-1(7-37)OH. Likewise, the first 31 amino acids of an extended GLP-1 analog has the amino acid sequence of GLP-1(7-37)OH or a fragment thereof, modified so that from one, two, three, four, five, or six amino acids differ from the amino acid in the corresponding position of GLP-1(7-37)OH or a fragment of GLP-1(7-37)OH.

In the nomenclature used herein to designate GLP-1 peptides, the substituting amino acid and its position is indicated prior to the parent structure. For example, Glu²²-GLP-1(7-37)OH designates a GLP-1 compound in which the glycine normally found at position 22 of GLP-1(7-37)OH has been replaced with glutamic acid; Val⁸-Glu²²-GLP-1(7-37)OH designates a GLP-1 compound in which alanine normally found at position 8 and glycine normally found at position 22 of GLP-1(7-37)OH have been replaced with valine and glutamic acid, respectively.

A “GLP-1 fragment” is a polypeptide obtained after truncation of one or more amino acids from the N-terminus and/or C-terminus of GLP-1(7-37)OH or a GLP-1(7-37)OH analog. The nomenclature used to describe GLP-1(7-37)OH carries over to GLP-1 fragments. For example, GLP-1(9-36)OH denotes a GLP-1 fragment obtained by truncating two amino acids from the N-terminus and one amino acid from the C-terminus. The amino acids in the fragment are denoted by the same number as the corresponding amino acid in GLP-1(7-37)OH, For example, the N-terminal glutamic acid in GLP-1(9-36)OH is at position 9; position 12 is occupied by phenylalanine; and position 22 is occupied by glycine, as in GLP-1(7-37)OH.

“Insulinotropic activity” refers to the ability to stimulate insulin secretion in response to elevated glucose levels, thereby causing glucose uptake by cells and decreased plasma glucose levels. Insulinotropic activity can be assessed by methods known in the art, including using in vivo experiments and in vitro assays that measure GLP-1 receptor binding activity or receptor activation, e.g., assays employing pancreatic islet cells or insulinoma cells, as described in EP 619,322 to Gelfand, et al., and U.S. Pat. No. 5,120,712, respectively. Insulinotropic activity is routinely measured in humans by measuring insulin levels or C-peptide levels.

Examples of non-naturally occurring amino acids include α-methyl amino acids (e.g., α-methyl alanine), D-amino acids, histidine-like amino acids (e.g., 2-amino-histidine, β-hydroxy-histidine, homohistidine, α-fluoromethyl-histidine and α-methyl-histidine), amino acids having an extra methylene in the side chain (“homo” amino acids) and amino acids in which a carboxylic acid functional group in the side chain is replaced with a sulfonic acid group (e.g., cysteic acid). Preferable non-natural amino acid analogs of cysteine include D-cysteine, homocysteine, or penicillamine. Preferably, however, the GLP-1 compounds of the present invention comprise only naturally occurring amino acids except as otherwise specifically provided herein.

As used herein, “reactive group” refers to chemical groups capable of forming a covalent bond. The term “linking group” refers to a chemical moiety that links or connects a reactive group to a GLP-1 peptide.

The term “orthogonal protecting group” as used herein refers to a protecting group on a synthetic peptide that is unique relative to the other protecting groups on the peptide, such that the orthogonal protecting group may be selectively removed while the other protecting groups remain attached to the peptide.

The term “blood component” as used herein refers to components in blood to which reactive groups in GLP-1 compounds can form covalent bonds. A blood component accordingly will contain a chemical group such as a thiol group, a hydroxyl group, or an amino group which can covalently bond to the reactive group of a GLP-1 compound of the present invention. Blood components include blood proteins, blood cells, and bodily tissues.

Blood components include both mobile or non-mobile blood proteins, cells, and tissues. Mobile blood components generally do not occupy a particular site for more than 5, and more typically, more than one minute. These mobile blood components remain in the blood for extended periods of time, having half-lives of about 12 or more hours. Such mobile blood components include serum albumin, transferrin, ferritin, and immunoglobulins. Non-mobile blood components include membrane receptors, interstitial proteins, fibrins, collagens, platelets, endothelial cells, epithelial cells, somatic cells, skeletal and smooth muscle cells, neuronal components, osteocytes, osteoclasts, and tissues, particularly those associated with the circulatory and lymphatic systems.

GLP-1 Peptides

The GLP-1 compounds of the present invention contain GLP-1 peptides that are modified through the attachment of a reactive group. A reactive group may be attached to a GLP-1 peptide at any of a number of sites on the peptide, including but not limited to lysine side chains, cysteine thiols, and carboxylic groups. Preferably, GLP-1 peptides to be modified at a lysine or cysteine and which terminate at position 37 respectively will have a lysine or cysteine at position 37 in the peptide. Accordingly, GLP-1 peptides of the present invention will include the GLP-1 peptides specified herein as well as the GLP-1 peptides that will result from substituting position 37 in these specified GLP-1 peptides with lysine or cysteine.

The GLP-1 peptides of the present invention typically have increased potency compared to Val⁸-GLP-1(7-37)OH. Native GLP-1(7-37)OH is rapidly degraded by dipeptidylamino-peptidase IV (DPP-IV) after injection and the half-life of GLP-1(7-37)OH is approximately five minutes. Analogs such as Val⁸-GLP-1(7-37)OH wherein the alanine at position 8 has been substituted with a different amino acid have been developed because these analogs are resistant to DPP-IV degradation and thus, have an increased half-life. However, these analogs are generally not potent enough to make administration by alternative delivery technology feasible on a commercial scale. Thus, Val⁸-GLP-1(7-37)OH is used as a comparator to illustrate the increased potency of the novel GLP-1 compounds encompassed by the present invention.

Preferably, the GLP-1 compounds of the present invention comprise GLP-1 analogs wherein the backbone for such analogs or fragments contains an amino acid other than alanine at position 8 (position 8 analogs). The backbone may also include L-histidine, D-histidine, or modified forms of histidine such as desamino-histidine, 2-amino-histidine, β-hydroxy-histidine, homohistidine, α-fluoromethyl-histidine, or α-methyl-histidine at position 7. It is preferable that these position 8 analogs contain one or more additional changes at positions 12, 16, 18, 19, 20, 22, 25, 27, 30, 33, and 37 compared to the corresponding amino acid of native GLP-1(7-37)OH. It is more preferable that these position 8 analogs contain one or more additional changes at positions 16, 18, 22, 25 and 33 compared to the corresponding amino acid of native GLP-1(7-37)OH.

In a preferred embodiment, the GLP-1 analog is GLP-1(7-37)OH wherein the amino acid at position 12 is selected from the group consisting of tryptophan or tyrosine. It is more preferred that in addition to the substitution at position 12, the amino acid at position 8 is substituted with glycine, valine, leucine, isoleucine, serine, threonine, or methionine and more preferably valine or glycine. It is even more preferred that in addition to the substitutions at position 12 and 8, the amino acid at position 22 is substituted with glutamic acid.

In another preferred embodiment, the GLP-1 analog is GLP-1(7-37)OH wherein the amino acid at position 16 is selected from the group consisting of tryptophan, isoleucine, leucine, phenylalanine, or tyrosine. It is more preferred that in addition to the substitution at position 16, the amino acid at position 8 is substituted with glycine, valine, leucine, isoleucine, serine, threonine, or methionine and more preferably valine or glycine. It is even more preferred that in addition to the substitutions at position 16 and 8, the amino acid at position 22 is substituted with glutamic acid. It is also preferred that in addition to the substitutions at positions 16 and 8, the amino acid at position 30 is substituted with glutamic acid. It is also preferred that in addition to the substitutions at positions 16 and 8, the amino acid at position 37 is substituted with histidine.

In another preferred embodiment, the GLP-1 analog is GLP-1(7-37)OH wherein the amino acid at position 18 is selected from the group consisting of tryptophan, tyrosine, phenylalanine, lysine, leucine, or isoleucine, preferably tryptophan, tyrosine, and isoleucine. It is more preferred that in addition to the substitution at position 18, the amino acid at position 8 is substituted with glycine, valine, leucine, isoleucine, serine, threonine, or methionine and more preferably valine or glycine. It is even more preferred that in addition to the substitutions at position 18 and 8, the amino acid at position 22 is substituted with glutamic acid. It is also preferred that in addition to the substitutions at positions 18 and 8, the amino acid at position 30 is substituted with glutamic acid. It is also preferred that in addition to the substitutions at positions 18 and 8, the amino acid at position 37 is substituted with histidine

In another preferred embodiment, the GLP-1 analog is GLP-1(7-37)OH wherein the amino acid at position 19 is selected from the group consisting of tryptophan or phenylalanine, preferably tryptophan. It is more preferred that in addition to the substitution at position 19, the amino acid at position 8 is substituted with glycine, valine, leucine, isoleucine, serine, threonine, or methionine and more preferably valine or glycine. It is even more preferred that in addition to the substitutions at position 19 and 8, the amino acid at position 22 is substituted with glutamic acid. It is also preferred that in addition to the substitutions at positions 19 and 8, the amino acid at position 30 is substituted with glutamic acid. It is also preferred that in addition to the substitutions at positions 19 and 8, the amino acid at position 37 is substituted with histidine

In another preferred embodiment, the GLP-1 analog is GLP-1(7-37)OH wherein the amino acid at position 20 is phenylalanine, tyrosine, or tryptophan. It is more preferred that in addition to the substitution at position 20, the amino acid at position 8 is substituted with glycine, valine, leucine, isoleucine, serine, threonine, or methionine and more preferably valine or glycine. It is even more preferred that in addition to the substitutions at position 20 and 8, the amino acid at position 22 is substituted with glutamic acid. It is also preferred that in addition to the substitutions at positions 20 and 8, the amino acid at position 30 is substituted with glutamic acid. It is also preferred that in addition to the substitutions at positions 20 and 8, the amino acid at position 37 is substituted with histidine

In another preferred embodiment, the GLP-1 analog is GLP-1(7-37)OH wherein the amino acid at position 25 is selected from the group consisting of valine, isoleucine, and leucine, preferably valine. It is more preferred that in addition to the substitution at position 25, the amino acid at position 8 is substituted with glycine, valine, leucine, isoleucine, serine, threonine, or methionine and more preferably valine or glycine. It is even more preferred that in addition to the substitutions at position 25 and 8, the amino acid at position 22 is substituted with glutamic acid. It is also preferred that in addition to the substitutions at positions 25 and 8, the amino acid at position 30 is substituted with glutamic acid. It is also preferred that in addition to the substitutions at positions 25 and 8, the amino acid at position 37 is substituted with histidine.

In another preferred embodiment, the GLP-1 analog is GLP-1(7-37)OH wherein the amino acid at position 27 is selected from the group consisting of isoleucine or alanine. It is more preferred that in addition to the substitution at position 27, the amino acid at position 8 is substituted with glycine, valine, leucine, isoleucine, serine, threonine, or methionine and more preferably valine or glycine. It is even more preferred that in addition to the substitutions at position 27 and 8, the amino acid at position 22 is substituted with glutamic acid. It is also preferred that in addition to the substitutions at positions 27 and 8, the amino acid at position 30 is substituted with glutamic acid. It is also preferred that in addition to the substitutions at positions 27 and 8, the amino acid at position 37 is substituted with histidine

In another preferred embodiment, the GLP-1 analog is GLP-1(7-37)OH wherein the amino acid at position 33 is isoleucine. It is more preferred that in addition to the substitution at position 33, the amino acid at position 8 is substituted with glycine, valine, leucine, isoleucine, serine, threonine, or methionine and more preferably valine or glycine. It is even more preferred that in addition to the substitutions at position 33 and 8, the amino acid at position 22 is substituted with glutamic acid. It is also preferred that in addition to the substitutions at positions 33 and 8, the amino acid at position 30 is substituted with glutamic acid. It is also preferred that in addition to the substitutions at positions 33 and 8, the amino acid at position 37 is substituted with histidine

It is also preferable that the GLP-1 peptides of the present invention have other combinations of substituted amino acids. The present invention encompasses a GLP-1 peptide comprising the amino acid sequence of formula 1 (SEQ ID NO:1)

Formula 1

| | |
|---|---|
| Xaa₇-Xaa₈-Glu-Gly-Thr-Xaa₁₂-Thr-Ser- | (SEQ ID NO: 1) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Lys-Gly-Arg- | |
| Xaa₃₇ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val or Ile;
Xaa₃₇ is: L-cysteine, D-cysteine, homocysteine, or penicillamine;
   provided that the GLP-1 peptide does not have the sequence of GLP-1(7-37)OH, GLP-1(7-36)-NH₂, Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser⁸-GLP-1(7-37)OH, Ser⁸-GLP-1(7-36)NH₂, Thr⁸-GLP-1(7-37)OH, Thr⁸-GLP-1(7-36)NH₂, Val⁸-Tyr¹²-GLP-1(7-37)OH, Val⁸-Tyr¹²-GLP-1(7-36)NH₂, Val⁸-Tyr¹⁶-GLP-1(7-37)OH, Val⁸-Tyr¹⁶-GLP-1(7-36)NH₂, Val⁸-Glu²²-GLP-1(7-37)OH, Val⁸-Glu²²-GLP-1(7-36)NH₂, Gly⁸-Glu²²-GLP-1(7-37)OH, Gly⁸-Glu²²-GLP-1(7-36)NH₂, Val⁸-Asp²²-GLP-1(7-37)OH, Val⁸-Asp²²-GLP-1(7-36)NH₂, Gly⁸-Asp²²-GLP-1(7-37)OH, Gly⁸-Asp²²-GLP-1(7-36)NH₂, Val⁸-Lys²²-GLP-1(7-37)OH, Val⁸-Lys²²-GLP-1(7-36)NH₂, Gly⁸-Lys²²-GLP-1(7-37)OH, Gly⁸-Lys²²-GLP-1(7-36)NH₂, Leu⁸-Glu²²-GLP-1(7-37)OH, Leu⁸-Glu²²-GLP-1(7-36)NH₂, Ile⁸-Glu²²-GLP-1(7-37)OH, Ile⁸-Glu²²-GLP-1(7-36)NH₂, Leu⁸-Asp²²-GLP-1(7-37)OH, Leu⁸-Asp²²-GLP-1(7-36)NH₂, Ile⁸-Asp²²-GLP-1(7-37)OH, Ile⁸-Asp²²-GLP-1(7-36)NH₂, Leu⁸-Lys²²-GLP-1(7-37)OH, Leu⁸-Lys²²-GLP-1(7-36)NH₂, Ile⁸-Lys²²-GLP-1(7-37)OH, Ile⁸-Lys²²-GLP-1(7-36)NH₂, Ser⁸-Glu²²-GLP-1(7-37)OH, Ser⁸-Glu²²-GLP-1(7-36)NH₂, Thr⁸-Glu²²-GLP-1(7-37)OH, Thr⁸-Glu²²-GLP-1(7-36)NH₂, Ser⁸-Asp²²-GLP-1(7-37)OH, Ser⁸-Asp²²-GLP-1(7-36)NH₂, Thr⁸-Asp²²-GLP-1(7-37)OH, Thr⁸-Asp²²-GLP-1(7-36)NH₂, Ser⁸-Lys²²-GLP-1(7-37)OH, Ser⁸-Lys²²-GLP-1(7-36)NH₂, Thr⁸-Lys²²-GLP-1(7-37)OH, Thr⁸-Lys²²-GLP-1(7-36)NH₂, Glu²²-GLP-1(7-37)OH, Glu²²-GLP-1(7-36)NH₂, Asp²²-GLP-1(7-37)OH, Asp²²-GLP-1(7-36)NH₂, Lys²²-GLP-1(7-37)OH, Lys²²-GLP-1(7-36)NH₂, Val⁸-Ala²⁷-GLP-1(7-37)OH, Val⁸-Glu²²-Ala²⁷-GLP-1(7-37)OH, Val⁸-Glu³⁰-GLP-1(7-37)OH, Val⁸-Glu³⁰-GLP-1(7-36)NH₂, Gly⁸-Glu³⁰-GLP-1(7-37)OH, Gly⁸-Glu³⁰-GLP-1(7-36)NH₂, Leu⁸-Glu³⁰-GLP-1(7-37)OH, Leu⁸-Glu³⁰-GLP-1(7-36)NH₂, Ile⁸-Glu³⁰-GLP-1(7-37)OH, Ile⁸-Glu³⁰-GLP-1(7-36)NH₂, Ser⁸-Glu³⁰-GLP-1(7-37)OH, Ser⁸-Glu³⁰-GLP-1(7-36)NH₂, Thr⁸-Glu³⁰-GLP-1(7-37)OH, Thr⁸-Glu³⁰-GLP-1(7-36)NH₂, Val⁸-His³⁷-GLP-1(7-37)OH, Val⁸-His³⁷-GLP-1(7-36)NH₂, Gly⁸-His³⁷-GLP-1(7-37)OH, Gly⁸-His³⁷-GLP-1(7-36)NH₂, Leu⁸-His³⁷-GLP-1(7-37)OH, Leu⁸-His³⁷ -GLP-1(7-36)NH_{2,} Ile⁸-His³⁷-GLP-1(7-37)OH, Ile⁸-His³⁷-GLP-1(7-36)NH_{2,} Ser⁸-His³⁷-GLP-1(7-37)OH, Ser¹-His³⁷-GLP-1(7-36)NH_{2,} Thr⁸-His³⁷-GLP-1(7-37)OH, Thr⁸-His³⁷-GLP-1(7-36)NH₂.

The present invention also encompasses a GLP-1 peptide comprising the amino acid sequence of formula 2 (SEQ ID) NO:2)

Formula 2

| | |
|---|---|
| Xaa₇-Xaa₈-Glu-Gly-Thr-Phe-Thr-Ser- | (SEQ ID NO:2) |
| Asp-Xaa₁₆-Ser-Xaa₁₈-Tyr-Leu-Glu- | |
| Xaa₂₂-Gln-Ala-Xaa₂₅-Lys-Glu-Phe-Ile- | |
| Ala-Trp-Leu-Xaa₃₃-Lys-Gly-Arg-Xaa₃₇ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Gly, Ala, Val, Leu, Ile, Ser, or Thr;
Xaa₁₆ is: Val, Phe, Tyr, or Trp;
Xaa₁₈ is: Ser, Tyr, Trp, Phe, Lys, Ile, Leu, or Val;
Xaa₂₂ is: Gly, Glu, Asp, or Lys;
Xaa₂₅ is: Ala, Val, Ile, or Leu;
Xaa₃₃ is: Val or Ile; and
Xaa₃₇ is: L-cysteine, D-cysteine, homocysteine, or penicillamine;
   provided that the GLP-1 peptide does not have the sequence of GLP-1(7-37)OH, GLP-1(7-36)-NH₂, Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser⁸-GLP-1(7-37)OH, Ser⁸-GLP-1(7-36)NH₂, Thr⁸-GLP-1(7-37)OH, Thr⁸-GLP-1(7-36)NH₂, Val⁸-Tyr¹⁶-GLP-1(7-37)OH, Val⁸-Tyr¹⁶-GLP-1(7-36)NH₂, Val⁸-Glu²²-GLP-1(7-37)OH, Val⁸-Glu²²-GLP-1(7-36)NH₂, Gly⁸-Glu²²-GLP-1(7-37)OH, Gly⁸-Glu²²-GLP-1(7-36)NH₂, Val⁸-Asp²²-GLP-1(7-37)OH, Val⁸-Asp²²-GLP-1(7-36)NH₂, Gly⁸-Asp²²-GLP-1(7-37)OH, Gly⁸-Asp²²-GLP-1(7-36)NH₂, Val⁸-Lys²²-GLP-1(7-37)OH, Val⁸-Lys²²-GLP-1(7-36)NH₂, Gly⁸-Lys²²-GLP-1(7-37)OH, Gly⁸-Lys²²-GLP-1(7-36)NH₂, Leu⁸-Glu²²-GLP-1(7-37)OH, Leu⁸-Glu²²-GLP-1(7-36)NH₂, Ile⁸-Glu²²-GLP-1(7-37)OH, Ile⁸-Glu²²-GLP-1(7-36)NH₂, Leu⁸-Asp²²-GLP-1(7-37)OH, Leu⁸-Asp²²-GLP-1(7-36)NH₂, Ile⁸-Asp²²-GLP-1(7-37)OH, Ile⁸-Asp²²-GLP-1(7-36)NH₂, Leu⁸-Lys²²-GLP-1(7-37)OH, Leu⁸-Lys²²-GLP-1(7-36)NH₂, Ile⁸-Lys²²-GLP-1(7-37)OH, Ile⁸-Lys²²-GLP-1(7-36)NH₂, Ser⁸-Glu²²-GLP-1(7-37)OH, Ser⁸-Glu²²-GLP-1(7-36)NH₂, Thr⁸-Glu²²-GLP-1(7-37)OH, Thr⁸-Glu²²-GLP-1(7-36)NH₂, Ser⁸-Asp²²-GLP-1(7-37)OH, Ser⁸-Asp²²-GLP-1(7-36)NH₂, Thr⁸-Asp²²-GLP-1(7-37)OH, Thr⁸-Asp²²-GLP-1(7-36)NH₂, Ser⁸-Lys²²-GLP-1(7-37)OH, Ser⁸-Lys²²-GLP-1(7-36)NH₂, Thr⁸-Lys²²-GLP-1(7-37)OH, Thr⁸-Lys²²-GLP-1(7-36)NH₂, Glu²²-GLP-1(7-37)OH, Glu²²-GLP-1(7-36)NH₂, Asp²²-GLP-1(7-37)OH, Asp²²-GLP-1(7-36)NH₂, Lys²²-GLP-1(7-37)OH, Lys²²-GLP-1(7-36)NH₂.

The present invention further encompasses a GLP-1 peptide comprising the amino acid sequence of formula 8 (SEQ ID NO:8)

Formula 8

| | |
|---|---|
| Xaa₇-Xaa₈-Glu-Gly-Thr-Xaa₁₂-Thr-Ser- | (SEQ ID NO:8) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Lys-Gly-Arg- | |
| Lys | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, Lys;
Xaa₂₅ is: Ala, Val, Ile, or Leu;
Xaa₂₇ is: Glu, Ile, or Ala;
Xaa₃₀ is: Ala or Glu; and
Xaa₃₃ is: Val, or Ile;
   provided that the GLP-1 peptide does not have the sequence of GLP-1(7-37)OH, GLP-1(7-36)-NH₂, Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser⁸-GLP-1(7-37)OH, Ser⁸-GLP-1(7-36)NH₂, Thr⁸-GLP-1(7-37)OH, Thr⁸-GLP-1(7-36)NH₂, Val⁸-Tyr¹²-GLP-1(7-37)OH, Val⁸-Tyr¹²-GLP-1(7-36)NH₂, Val⁸-Tyr¹⁶-GLP-1(7-37)OH, Val⁸-Tyr¹⁶-GLP-1(7-36)NH₂, Val⁸-Glu²²-GLP-1(7-37)OH, Val⁸-Glu²²-GLP-1(7-36)NH₂, Gly⁸-Glu²²-GLP-1(7-37)OH, Gly⁸-Glu²²-GLP-1(7-36)NH₂, Val⁸-Asp²²-GLP-1(7-37)OH, Val⁸-Asp²²-GLP-1(7-36)NH₂, Gly⁸-Asp²²-GLP-1(7-37)OH, Gly⁸-Asp²²-GLP-1(7-36)NH₂, Val⁸-Lys²²-GLP-1(7-37)OH, Val⁸-Lys²²-GLP-1(7-36)NH₂, Gly⁸-Lys²²-GLP-1(7-37)OH, Gly⁸-Lys²²-GLP-1(7-36)NH₂, Leu⁸-Glu²²-GLP-1(7-37)OH, Leu⁸-Glu²²-GLP-1(7-36)NH₂, Ile⁸-Glu²²-GLP-1(7-37)OH, Ile⁸-Glu²²-GLP-1(7-36)NH₂, Leu⁸-Asp²²-GLP-1(7-37)OH, Leu⁸-Asp²²-GLP-1(7-36)NH₂, Ile⁸-Asp²²-GLP-1(7-37)OH, Ile⁸-Asp²²-GLP-1(7-36)NH₂, Leu⁸-Lys²²-GLP-1(7-37)OH, Leu⁸-Lys²²-GLP-1(7-36)NH₂, Ile⁸-Lys²²-GLP-1(7-37)OH, Ile⁸-Lys²²-GLP-1(7-36)NH₂, Ser⁸-Glu²²-GLP-1(7-37)OH, Ser⁸-Glu²²-GLP-1(7-36)NH₂, Thr⁸-Glu²²-GLP-1(7-37)OH, Thr⁸-Glu²²-GLP-1(7-36)NH₂, Ser⁸-Asp²²-GLP-1(7-37)OH, Ser⁸-Asp²²-GLP-1(7-36)NH₂, Thr⁸-Asp²²-GLP-1(7-37)OH, Thr⁸-Asp²²-GLP-1(7-36)NH₂, Ser⁸-Lys²²-GLP-1(7-37)OH, Ser⁸-Lys²²-GLP-1(7-36)NH₂, Thr⁸-Lys²²-GLP-1(7-37)OH, Thr⁸-Lys²²-GLP-1(7-36)NH₂, Glu²²-GLP-1(7-37)OH, Glu²²-GLP-1(7-36)NH₂, Asp²²-GLP-1(7-37)OH, Asp²²-GLP-1(7-36)NH₂, Lys²²-GLP-1(7-37)OH, Lys²²-GLP-1(7-36)NH₂, Val⁸-Ala²⁷-GLP-1(7-37)OH, Val⁸-Glu²²-Ala²⁷-GLP-1(7-37)OH, Val⁸-Glu³⁰-GLP-1(7-37)OH, Val⁸-Glu³⁰-GLP-1(7-36)NH₂, Gly⁸-Glu³⁰-GLP-1(7-37)OH, Gly⁸-Glu³⁰-GLP-1(7-36)NH₂, Leu⁸-Glu³⁰-GLP-1(7-37)OH, Leu⁸-Glu³⁰-GLP-1(7-36)NH₂, Ile⁸-Glu³⁰-GLP-1(7-37)OH, Ile⁸-Glu³⁰-GLP-1(7-36)NH₂, Ser⁸-Glu³⁰-GLP-1(7-37)OH, Ser⁸-Glu³⁰-GLP-1(7-36)NH₂, Thr⁸-Glu³⁰-GLP-1(7-37)OH, Thr⁸-Glu³⁰-GLP-1(7-36)NH₂, Val⁸-His³⁷-GLP-1(7-37)OH, Val⁸-His³⁷-GLP-1(7-36)NH_{2,} Gly⁸-His³⁷-GLP-1(7-37)OH, Gly⁸-His³⁷-GLP-1(7-36)NH₂, Leu⁸-His³⁷-GLP-1(7-37)OH, Leu⁸-His³⁷-GLP-1(7-36)NH_{2,} Ile⁸-His³⁷-GLP-1(7-37)OH, Ile⁸-His³⁷-GLP-1(7-36)NH_{2,} Ser⁸-His³⁷-GLP-1(7-37)OH, Ser⁸-His³⁷-GLP-1(7-36)NH_{2,} Thr⁸-His³⁷-GLP-1(7-37)OH, Thr⁸-His³⁷-GLP-1(7-36)NH₂, Lys³⁷-GLP-1(7-37)OH.

The present invention also encompasses a GLP-1 peptide comprising the amino acid sequence of formula 9 (SEQ ID NO:9)

Formula 9

| | |
|---|---|
| Xaa₇-Xaa₈-Glu-Gly-Thr-Phe-Thr-Ser- | (SEQ ID NO:9) |
| Asp-Xaa₁₆-Ser-Xaa₁₈-Tyr-Leu-Glu- | |
| Xaa₂₂-Gln-Ala-Xaa₂₅-Lys-Glu-Phe-Ile- | |
| Ala-Trp-Leu-Xaa₃₃-Lys-Gly-Arg-Lys | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Gly, Ala, Val, Leu, Ile, Ser, or Thr;
Xaa₁₆ is: Val, Phe, Tyr, or Trp;
Xaa₁₈ is: Ser, Tyr, Trp, Phe, Lys, Ile, Leu, or Val;
Xaa₂₂ is: Gly, Glu, Asp, or Lys;
Xaa₂₅ is: Ala, Val, Ile, or Leu; and
Xaa₃₃ is: Val or Ile;
   provided that the GLP-1 peptide does not have the sequence of GLP-1(7-37)OH, GLP-1(7-36)-NH₂, Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser⁸-GLP-1(7-37)OH, Ser⁸-GLP-1(7-36)NH₂, Thr⁸-GLP-1(7-37)OH, Thr⁸-GLP-1(7-36)NH₂, Val⁸-Tyr¹²-GLP-1(7-37)OH, Val⁸-Tyr¹²-GLP-1(7-36)NH₂, Val⁸-Tyr¹⁶-GLP-1(7-37)OH, Val⁸-Tyr¹⁶-GLP-1(7-36)NH₂, Val⁸-Glu²²-GLP-1(7-37)OH, Val⁸-Glu²²-GLP-1(7-36)NH₂, Gly⁸-Glu²²-GLP-1(7-37)OH, Gly⁸-Glu²²-GLP-1(7-36)NH₂, Val⁸-Asp²²-GLP-1(7-37)OH, Val⁸-Asp²²-GLP-1(7-36)NH₂, Gly⁸-Asp²²-GLP-1(7-37)OH, Gly⁸-Asp²²-GLP-1(7-36)NH₂, Val⁸-Lys²²-GLP-1(7-37)OH, Val⁸-Lys²²-GLP-1(7-36)NH₂, Gly⁸-Lys²²-GLP-1(7-37)OH, Gly⁸-Lys²²-GLP-1(7-36)NH₂, Leu⁸-Glu²²-GLP-1(7-37)OH, Leu⁸-Glu²²-GLP-1(7-36)NH₂, Ile⁸-Glu²²-GLP-1(7-37)OH, Ile⁸-Glu²²-GLP-1(7-36)NH₂, Leu⁸-Asp²²-GLP-1(7-37)OH, Leu⁸-Asp²²-GLP-1(7-36)NH₂, Ile⁸-Asp²²-GLP-1(7-37)OH, Ile⁸-Asp²²-GLP-1(7-36)NH₂, Leu⁸-Lys²²-GLP-1(7-37)OH, Leu⁸-Lys²²-GLP-1(7-36)NH₂, Ile⁸-Lys²²-GLP-1(7-37)OH, Ile⁸-Lys²²-GLP-1(7-36)NH₂, Ser⁸-Glu²²-GLP-1(7-37)OH, Ser⁸-Glu²²-GLP-1(7-36)NH₂, Thr⁸-Glu²²-GLP-1(7-37)OH, Thr⁸-Glu²²-GLP-1(7-36)NH₂, Ser⁸-Asp²²-GLP-1(7-37)OH, Ser⁸-Asp²²-GLP-1(7-36)NH₂, Thr⁸-Asp²²-GLP-1(7-37)OH, Thr⁸-Asp²²-GLP-1(7-36)NH₂, Ser⁸-Lys²²-GLP-1(7-37)OH, Ser⁸-Lys²²-GLP-1(7-36)NH₂, Thr⁸-Lys²²-GLP-1(7-37)OH, Thr⁸-Lys²²-GLP-1(7-36)NH₂, Glu²²-GLP-1(7-37)OH, Glu²²-GLP-1(7-36)NH₂, Asp²²-GLP-1(7-37)OH, Asp²²-GLP-1(7-36)NH₂, Lys²²-GLP-1(7-37)OH, Lys²²-GLP-1(7-36)NH₂, Val⁸-Ala²⁷-GLP-1(7-37)OH, Val⁸-Glu²²-Ala²⁷-GLP-1(7-37)OH, Val⁸-Glu³⁰-GLP-1(7-37)OH, Val⁸-Glu³⁰-GLP-1(7-36)NH₂, Gly⁸-Glu³⁰-GLP-1(7-37)OH, Gly⁸-Glu³⁰-GLP-1(7-36)NH₂, Leu⁸-Glu³⁰-GLP-1(7-37)OH, Leu⁸-Glu³⁰-GLP-1(7-36)NH₂, Ile⁸-Glu³⁰-GLP-1(7-37)OH, Ile⁸-Glu³⁰-GLP-1(7-36)NH₂, Ser⁸-Glu³⁰-GLP-1(7-37)OH, Ser⁸-Glu³⁰-GLP-1(7-36)NH₂, Thr⁸-Glu³⁰-GLP-1(7-37)OH, Thr⁸-Glu³⁰-GLP-1(7-36)NH₂, Val⁸-His³⁷-GLP-1(7-37)OH, Val⁸-His³⁷-GLP-1(7-36)NH_{2,} Gly⁸-His³⁷-GLP-1(7-37)OH, Gly⁸-His³⁷-GLP-1(7-36)NH_{2,} Leu⁸-His³⁷-GLP-1(7-37)OH, Leu⁸-His³⁷-GLP-1(7-36)NH_{2,} Ile⁸-His³⁷-GLP-1(7-37)OH, Ile⁸-His³⁷-GLP-1(7-36)NH_{2,} Ser⁸-His³⁷-GLP-1(7-37)OH, Ser⁸-His³⁷-GLP-1(7-36)NH₂, Thr⁸-His³⁷-GLP-1(7-37)OH, Thr⁸-His³⁷-GLP-1(7-36)NH₂, Lys³⁷-GLP-1(7-37)OH.

The present invention further encompasses a GLP-1 peptide comprising the amino acid sequence of formula 15 (SEQ ID NO:15)

Formula 15

| | |
|---|---|
| Xaa₇-Xaa₈-Glu-Gly-Thr-Xaa₁₂-Thr-Ser- | (SEQ ID NO:15) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Lys-Gly-Arg- | |
| Xaa₃₇ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val, or Ile; and
Xaa₃₇ is: Gly, His, Lys, L-cysteine, D-cysteine, homocysteine, penicillamine, or NH₂, or is absent,
   provided that the GLP-1 peptide does not have the sequence of GLP-1(7-37)OH, GLP-1(7-36)-NH₂, Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser⁸-GLP-1(7-37)OH, Ser⁸-GLP-1(7-36)NH₂, Thr⁸-GLP-1(7-37)OH, Thr⁸-GLP-1(7-36)NH₂, Val⁸-Tyr¹²-GLP-1(7-37)OH, Val⁸-Tyr¹²-GLP-1(7-36)NH₂, Val⁸-Tyr¹⁶-GLP-1(7-37)OH, Val⁸-Tyr¹⁶-GLP-1(7-36)NH₂, Val⁸-Glu²²-GLP-1(7-37)OH, Val⁸-Glu²²-GLP-1(7-36)NH₂, Gly⁸-Glu²²-GLP-1(7-37)OH, Gly⁸-Glu²²-GLP-1(7-36)NH₂, Val⁸-Asp²²-GLP-1(7-37)OH, Val⁸-Asp²²-GLP-1(7-36)NH₂, Gly⁸-Asp²²-GLP-1(7-37)OH, Gly⁸-Asp²²-GLP-1(7-36)NH₂, Val⁸-Lys²²-GLP-1(7-37)OH, Val⁸-Lys²²-GLP-1(7-36)NH₂, Gly⁸-Lys²²-GLP-1(7-37)OH, Gly⁸-Lys²²-GLP-1(7-36)NH₂, Leu⁸-Glu²²-GLP-1(7-37)OH, Leu⁸-Glu²²-GLP-1(7-36)NH₂, Ile⁸-Glu²²-GLP-1(7-37)OH, Ile⁸-Glu²²-GLP-1(7-36)NH₂, Leu⁸-Asp²²-GLP-1(7-37)OH, Leu⁸-Asp²²-GLP-1(7-36)NH₂, Ile⁸-Asp²²-GLP-1(7-37)OH, Ile⁸-Asp²²-GLP-1(7-36)NH₂, Leu⁸-Lys²²-GLP-1(7-37)OH, Leu⁸-Lys²²-GLP-1(7-36)NH₂, Ile⁸-Lys²²-GLP-1(7-37)OH, Ile⁸-Lys²²-GLP-1(7-36)NH₂, Ser⁸-Glu²²-GLP-1(7-37)OH, Ser⁸-Glu²²-GLP-1(7-36)NH₂, Thr⁸-Glu²²-GLP-1(7-37)OH, Thr⁸-Glu²²-GLP-1(7-36)NH₂, Ser⁸-Asp²²-GLP-1(7-37)OH, Ser⁸-Asp²²-GLP-1(7-36)NH₂, Thr⁸-Asp²²-GLP-1(7-37)OH, Thr⁸-Asp²²-GLP-1(7-36)NH₂, Ser⁸-Lys²²-GLP-1(7-37)OH, Ser⁸-Lys²²-GLP-1(7-36)NH₂, Thr⁸-Lys²²-GLP-1(7-37)OH, Thr⁸-Lys²²-GLP-1(7-36)NH₂, Glu²²-GLP-1(7-37)OH, Glu²²-GLP-1(7-36)NH₂, Asp²²-GLP-1(7-37)OH, Asp²²-GLP-1(7-36)NH₂, Lys²²-GLP-1(7-37)OH, Lys²²-GLP-1(7-36)NH₂, Val⁸-Ala²⁷-GLP-1(7-37)OH, Val⁸-Glu²²-Ala²⁷-GLP-1(7-37)OH, Val⁸-Glu³⁰-GLP-1(7-37)OH, Val⁸-Glu³⁰-GLP-1(7-36)NH₂, Gly⁸-Glu³⁰-GLP-1(7-37)OH, Gly⁸-Glu³⁰-GLP-1(7-36)NH₂, Leu⁸-Glu³⁰-GLP-1(7-37)OH, Leu⁸-Glu³⁰-GLP-1(7-36)NH₂, Ile⁸-Glu³⁰-GLP-1(7-37)OH, Ile⁸-Glu³⁰-GLP-1(7-36)NH₂, Ser⁸-Glu³⁰-GLP-1(7-37)OH, Ser⁸-Glu³⁰-GLP-1(7-36)NH₂, Thr⁸-Glu³⁰-GLP-1(7-37)OH, Thr⁸-Glu³⁰-GLP-1(7-36)NH₂, Val⁸-His³⁷-GLP-1(7-37)OH, Val⁸-His³⁷-GLP-1(7-36)NH_{2,} Gly⁸-His³⁷-GLP-1(7-37)OH, Gly⁸-His³⁷-GLP-1(7-36)NH_{2,} Leu⁸-His³⁷-GLP-1(7-37)OH, Leu⁸-His³⁷-GLP-1(7-36)NH_{2,} Ile⁸-His³⁷-GLP-1(7-37)OH, Ile⁸-His³⁷-GLP-1(7-36)NH_{2,} Ser⁸-His³⁷-GLP-1(7-37)OH, Ser⁸-His³⁷-GLP-1(7-36)NH_{2,} Thr⁸-His³⁷-GLP-1(7-37)OH, Thr⁸-His³⁷-GLP-1(7-36)NH₂, Lys³⁷-GLP-1(7-37)OH.

It is preferable that the GLP-1 peptides of formula 1, 2, 8, 9, and 15 have 6 or fewer changes compared to the corresponding amino acids in native GLP-1(7-37)OH. More preferred analogs have 5 or fewer changes compared to the corresponding amino acids in native GLP-1(7-37)OH or have 4 or fewer changes compared to the corresponding amino acids in native GLP-1(7-37)OH or have 3 changes compared to the corresponding amino acids in native GLP-1(7-37)OH.

Some preferred GLP-1 peptides of formula 1, 2, 8, 9, and 15 having multiple substitutions include GLP-1(7-37)OH wherein position 8 is valine or glycine, position 22 is glutamic acid, position 16 is tyrosine, leucine or tryptophan, position 18 is tyrosine, tryptophan, or isoleucine, position 25 is valine and position 33 is isoleucine. Other preferred GLP-1 compounds include the following: Val⁸-Tyr¹⁶-GLP-1(7-37)OH, Val⁸-Tyr¹²-Glu²²-GLP-1(7-37)OH, Val⁸-Tyr¹⁶-Phe¹⁹-GLP-1(7-37)OH, Val⁸-Tyr¹⁶-Glu²²-GLP-1(7-37)OH, Val⁸-Trp¹⁶-Glu²²-GLP-1(7-37)OH, Val⁸-Leu¹⁶-Glu²²-GLP-1(7-37)OH, Val⁸-Ile¹⁶-Glu²²-GLP-1(7-37)OH, Val⁸-Phe¹⁶-Glu²²-GLP-1(7-37)OH, Val⁸-Trp¹⁸-Glu²²-GLP-1(7-37)OH, Val⁸-Tyr¹⁸-Glu²²-GLP-1(7-37)OH, Val⁸-Phe¹⁸-Glu²²-GLP-1(7-37)OH, and Val⁸-Ile¹⁸-Glu²²-GLP-1(7-37)OH.

The GLP-1 compounds of the present invention further comprise extended GLP-1 peptides that are modified through the attachment of a reactive group. A reactive group may be attached to an extended GLP-1 peptide at any of a number of sites on the peptide, including but not limited to lysine side chains, cysteine thiols, and carboxylic groups.

The extended GLP-1 peptides of the present invention have one or more changes selected from the following positions relative to GLP-1(7-37): 7, 8, 12, 16, 18, 19, 20, 22, 25, 27, 30, 33, 34, 36, and 37. In addition, these extended GLP-1 peptides have between 1 and 14 amino acids added after amino acid residue number 37, which are designated amino acid positions 38-51 (Xaa₃₈ through Xaa₅₁). The C-terminal amino acid of an extended GLP-1 peptide thus may occur at any of positions 38-51. As used herein, the terminology “any of positions 38 through 51” will collectively refer to the additional amino acids of all extended GLP-1 peptides, which will have varying lengths of additional amino acids at the C-terminus relative to GLP-1 (7-37)OH. For example, reference to a “lysine at any of positions 37-51” will encompass having a lysine at any of positions 37-38, 37-45, or 37-51 in extended GLP-1 peptides that terminate at positions 38, 45, or 51, respectively.

Extended GLP-1 peptides to be modified at a lysine will contain a lysine at any of positions 37 through 51. While more than one lysine may be present in the peptide, only one lysine at any of positions 37 through 51 will be modified. Preferably, GLP-1 peptides to be modified at a lysine will contain a single lysine at any of positions 37 through 51.

Extended GLP-1 peptides to be modified at a cysteine will contain a single cysteine which occurs at any of positions 37 through 51. The single cysteine may be L-cysteine, or alternatively, may be a cysteine analog, such as D-cysteine, homocysteine, or penicillamine.

Extended GLP-1 peptides to be modified at a lysine or cysteine respectively will have a lysine or cysteine at any of positions 37 through 51 in the peptide. Accordingly, extended GLP-1 peptides of the present invention will include the extended GLP-1 peptides specified herein as well as the extended GLP-1 peptides will result from substituting any of positions 37 through 51 in these specified extended GLP-1 peptides with lysine or cysteine.

The present invention encompasses extended GLP-1 peptides comprising any combination of the amino acids provided in formulas 3 (SEQ ID NO:3), 6 (SEQ ID NO:6), 10 (SEQ ID NO:10), or formula 13 (SEQ ID NO:13) wherein these extended GLP-1 peptides exhibit insulinotropic activity.

Preferably, the extended GLP-1 peptides of the present invention comprise extended GLP-1 analogs wherein the backbone for such analogs or fragments contains an amino acid other than alanine at position 8 (position 8 analogs). The backbone may also include L-histidine, D-histidine, or modified forms of histidine such as desamino-histidine, 2-amino-histidine, β-hydroxy-histidine, homohistidine, α-fluoromethyl-histidine, or α-methyl-histidine at position 7. It is preferable that these position 8 analogs contain one or more additional changes at positions 12, 16, 18, 19, 20, 22, 25, 27, 30, 33, 34, 36, and 37 compared to the corresponding amino acid of native GLP-1(7-37). It is more preferable that these position 8 analogs contain one or more additional changes at positions 16, 18, 22, 25 and 33 compared to the corresponding amino acid of native GLP-1(7-37).

In a preferred embodiment, the amino acid at position 12 of an extended GLP-1 peptide is selected from the group consisting of tryptophan or tyrosine. It is more preferred that in addition to the substitution at position 12, the amino acid at position 8 is substituted with glycine, valine, leucine, isoleucine, serine, threonine, or methionine and more preferably valine or glycine. It is even more preferred that in addition to the substitutions at position 12 and 8, the amino acid at position 22 is substituted with glutamic acid.

In another preferred embodiment, the amino acid at position 16 of an extended GLP-1 peptide is selected from the group consisting of tryptophan, isoleucine, leucine, phenylalanine, or tyrosine. It is preferred that the amino acid at position 16 is tryptophan. It is more preferred that in addition to the substitutions at position 16, the amino acid at position 8 is substituted with glycine, valine, leucine, isoleucine, serine, threonine, or methionine and more preferably valine or glycine. It is even more preferred that in addition to the substitutions at position 16 and 8, the amino acid at position 22 is substituted with glutamic acid. It is also preferred that in addition to the substitutions at positions 16 and 8, the amino acid at position 33 is substituted with isoleucine. It is also preferred that in addition to the substitutions at position 8, 16, and 22, the amino acid at position 36 is substituted with glycine and the amino acid at position 37 is substituted with proline.

In another preferred embodiment, the amino acid at position 18 of an extended GLP-1 peptide is selected from the group consisting of tryptophan, tyrosine, phenylalanine, lysine, leucine, or isoleucine, preferably tryptophan, tyrosine, and isoleucine. It is more preferred that in addition to the substitution at position 18, the amino acid at position 8 is substituted with glycine, valine, leucine, isoleucine, serine, threonine, or methionine and more preferably valine or glycine. It is even more preferred that in addition to the substitutions at position 18 and 8, the amino acid at position 22 is substituted with glutamic acid. It is also preferred that in addition to the substitutions at positions 18 and 8, the amino acid at position 33 is substituted with isoleucine. It is also preferred that in addition to the substitutions at position 8, 18, and 22, the amino acid at position 36 is substituted with glycine and the amino acid at position 37 is substituted with proline.

In another preferred embodiment, the amino acid at position 19 of an extended GLP-1 peptide is selected from the group consisting of tryptophan or phenylalanine, preferably tryptophan. It is more preferred that in addition to the substitution at position 19, the amino acid at position 8 is substituted with glycine, valine, leucine, isoleucine, serine, threonine, or methionine and more preferably valine or glycine. It is even more preferred that in addition to the substitutions at position 19 and 8, the amino acid at position 22 is substituted with glutamic acid. It is also preferred that in addition to the substitutions at position 8, 19, and 22, the amino acid at position 36 is substituted with glycine and the amino acid at position 37 is substituted with proline.

In another preferred embodiment, the amino acid at position 20 of an extended GLP-1 peptide is selected from the group consisting of phenylalanine, tyrosine, or tryptophan, preferably tryptophan. It is more preferred that in addition to the substitution at position 20, the amino acid at position 8 is substituted with glycine, valine, leucine, isoleucine, serine, threonine, or methionine and more preferably valine or glycine. It is even more preferred that in addition to the substitutions at position 20 and 8, the amino acid at position 22 is substituted with glutamic acid. It is also preferred that in addition to the substitutions at position 8, 20, and 22, the amino acid at position 36 is substituted with glycine and the amino acid at position 37 is substituted with proline.

In another preferred embodiment, the amino acid at position 25 of an extended GLP-1 peptide is selected from the group consisting of valine, isoleucine, and leucine, preferably valine. It is more preferred that in addition to the substitution at position 25, the amino acid at position 8 is substituted with glycine, valine, leucine, isoleucine, serine, threonine, or methionine and more preferably valine or glycine. It is even more preferred that in addition to the substitutions at position 25 and 8, the amino acid at position 22 is substituted with glutamic acid. It is also preferred that in addition to the substitutions at position 8, 22, and 25, the amino acid at position 36 is substituted with glycine and the amino acid at position 37 is substituted with proline.

In another preferred embodiment, the amino acid at position 27 of an extended GLP-1 peptide is selected from the group consisting of isoleucine or alanine. It is more preferred that in addition to the substitution at position 27, the amino acid at position 8 is substituted with glycine, valine, leucine, isoleucine, serine, threonine, or methionine and more preferably valine or glycine. It is even more preferred that in addition to the substitutions at position 27 and 8, the amino acid at position 22 is substituted with glutamic acid. It is also preferred that in addition to the substitutions at position 8, 22, and 27, the amino acid at position 36 is substituted with glycine and the amino acid at position 37 is substituted with proline.

In another preferred embodiment, the amino acid at position 33 of an extended GLP-1 peptide is isoleucine. It is more preferred that in addition to the substitution at position 33, the amino acid at position 8 is substituted with glycine, valine, leucine, isoleucine, serine, threonine, or methionine and more preferably valine or glycine. It is even more preferred that in addition to the substitutions at position 33 and 8, the amino acid at position 22 is substituted with glutamic acid. It is also preferred that in addition to the substitutions at position 8, 22, and 33 the amino acid at position 36 is substituted with glycine and the amino acid at position 37 is substituted with proline.

In another preferred embodiment, the amino acid at position 34 is aspartic acid. It is more preferred that in addition to the substitution at position 34, the amino acid at position 8 is substituted with glycine, valine, leucine, isoleucine, serine, threonine, or methionine and more preferably valine or glycine. It is even more preferred that in addition to the substitutions at position 34 and 8, the amino acid at position 22 is substituted with glutamic acid. It is also preferred that in addition to the substitutions at position 8, 22, and 34 the amino acid at position 36 is substituted with glycine and the amino acid at position 37 is substituted with proline.

The C-terminal extension portion fused to the GLP-1 analog backbones discussed above is at least 4 amino acids in length, preferably between 6 and 13 amino acids in length. Preferably, the extended GLP-1 peptides of the present invention have a serine, proline, or histidine at position 38; a serine, arginine, threonine, tryptophan, or lysine at position 39; a serine or glycine at position 40; an alanine, aspartic acid, arginine, glutamic acid, lysine or glycine at position 41; a proline or alanine at position 42; and a proline or alanine at position 43. Additional amino acids that may be added include a proline, serine, alanine, arginine, lysine, or histidine at position 44; a serine, histidine, proline, lysine or arginine at position 45; a histidine, serine, arginine, or lysine at position 46; a histidine, serine, arginine, or lysine at position 47, glycine or histidine at position 48, proline or histidine at position 49, and serine or histidine at position 50. Preferably, histidine is the C-terminal amino acid at either position 44, 45, 46, 47, 48, 49 or 50.

It is preferred that when Xaa₃₄ is aspartic acid, then Xaa₄₁ is arginine or lysine. It is also preferred that Xaa₃₉ is serine. It is also preferred that when Xaa₄₁ is aspartic acid or arginine, then Xaa₄₂, Xaa₄₃, and Xaa₄₄ are all proline. The C-terminal amino acid may be in the typical acid form or may be amidated.

It is also preferable that the extended GLP-1 peptides of the present invention have other combinations of substituted amino acids. The present invention encompasses an extended GLP-1 peptide comprising the amino acid sequence of formula 3 (SEQ ID NO:3)

Formula 3

| | |
|---|---|
| Xaa₇-Xaa₈-Glu-Gly-Thr-Xaa₁₂-Thr-Ser- | (SEQ ID NO:3) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Xaa₃₄-Gly- | |
| Xaa₃₆-Xaa₃₇-Xaa₃₈-Xaa₃₉-Xaa₄₀-Xaa₄₁- | |
| Xaa₄₂-Xaa₄₃-Xaa₄₄-Xaa₄₅-Xaa₄₆-Xaa₄₇- | |
| Xaa₄₈ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, or Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val or Ile;
Xaa₃₄ is: Lys, Asp, Arg, or Glu;
Xaa₃₆ is: Gly, Pro, or Arg;
Xaa₃₇ is: Gly, Pro, Ser, L-cysteine, D-cysteine, homocysteine, or penicillamine;
Xaa₃₈ is: Ser, Pro, His, L-cysteine, D-cysteine, homocysteine, penicillamine, NH₂;
Xaa₃₉ is: Ser, Arg, Thr, Trp, Lys, L-cysteine, D-cysteine, homocysteine, penicillamine, NH₂, or is absent;
Xaa₄₀ is: Ser, Gly, L-cysteine, D-cysteine, homocysteine, penicillamine, NH₂, or is absent;
Xaa₄₁ is: Ala, Asp, Arg, Glu, Lys, Gly, L-cysteine, D-cysteine, homocysteine, penicillamine, NH₂, or is absent;
Xaa₄₂ is: Pro, Ala, L-cysteine, D-cysteine, homocysteine, penicillamine, NH₂, or is absent;
Xaa₄₃ is: Pro, Ala, L-cysteine, D-cysteine, homocysteine, penicillamine, NH₂, or is absent;
Xaa₄₄ is: Pro, Ala, Arg, Lys, His, L-cysteine, D-cysteine, homocysteine, penicillamine, NH₂, or is absent;
Xaa₄₅ is: Ser, His, Pro, Lys, Arg, L-cysteine, D-cysteine, homocysteine, penicillamine, NH₂, or is absent;
Xaa₄₆ is: His, Ser, Arg, Lys, L-cysteine, D-cysteine, homocysteine, penicillamine, NH₂, or is absent;
Xaa₄₇ is: His, Ser, Arg, Lys, L-cysteine, D-cysteine, homocysteine, penicillamine, NH₂, or is absent; and
Xaa₄₈ is: L-cysteine, D-cysteine, homocysteine, penicillamine, NH₂, or is absent;

provided that if Xaa₃₉, Xaa₄₀, Xaa₄₁, Xaa₄₂, Xaa₄₃, Xaa₄₄, Xaa₄₅, Xaa₄₆, or Xaa₄₇ is absent each amino acid downstream is absent and further provided that the GLP-1 peptide does not have the following C-terminal amino acid extension beginning at Xaa₃₆: Gly-Pro-Ser-Ser-Gly-Ala-Pro-Pro-Pro-Ser-NH₂.

The present invention also encompasses an extended GLP-1 peptide comprising the amino acid sequence of formula 4 (SEQ ID NO:4)

Formula 4

| | |
|---|---|
| Xaa₇-Xaa₈-Glu-Gly-Thr-Phe-Thr-Ser- | (SEQ ID NO: 4) |
| Asp-Xaa₁₆-Ser-Ser-Tyr-Lys-Glu-Xaa₂₂- | |
| Gln-Ala-Xaa₂₅-Lys-Glu-Phe-Ile-Ala- | |
| Trp-Leu-Xaa₃₃-Xaa₃₄-Gly-Xaa₃₆-Xaa₃₇- | |
| Xaa₃₈-Xaa₃₉-Xaa₄₀-Xaa₄₁-Xaa₄₂-Xaa₄₃- | |
| Xaa₄₄-Xaa₄₅-Xaa₄₆-Xaa₄₇-Xaa₄₈ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Gly, Val, Leu, Ile, Ser, or Thr;
Xaa₁₆ is: Val, Trp, Ile, Trp, Phe, or Tyr;

Xaa₂₂ is: Gly, Glu, Asp, or Lys;
Xaa₂₅ is: Ala, Val, Ile, or Leu;
Xaa₃₃ is: Val or Ile;
Xaa₃₄ is: Lys, Asp, Arg, or Glu;
Xaa₃₆ is: Gly, Pro, or Arg;
Xaa₃₇ is: Gly, Pro, Ser, L-cysteine, D-cysteine, homocysteine, or penicillamine;
Xaa₃₈ is: Ser, Pro, His, L-cysteine, D-cysteine, homocysteine, penicillamine, NH₂, or is absent;
Xaa₃₉ is: Ser, Arg, Thr, Trp, Lys, L-cysteine, D-cysteine, homocysteine, penicillamine, NH₂, or is absent;
Xaa₄₀ is: Ser, Gly, L-cysteine, D-cysteine, homocysteine, penicillamine, NH₂, or is absent;
Xaa₄₁ is: Ala, Asp, Arg, Glu, Lys, Gly, L-cysteine, D-cysteine, homocysteine, penicillamine, NH₂, or is absent;
Xaa₄₂ is: Pro, Ala, L-cysteine, D-cysteine, homocysteine, penicillamine, NH₂, or is absent;
Xaa₄₃ is: Pro, Ala, L-cysteine, D-cysteine, homocysteine, penicillamine, NH₂, or is absent;
Xaa₄₄ is: Pro, Ala, Arg, Lys, His, L-cysteine, D-cysteine, homocysteine, penicillamine, NH₂, or is absent;
Xaa₄₅ is: Ser, His, Pro, Lys, Arg, L-cysteine, D-cysteine, homocysteine, penicillamine, NH₂, or is absent;
Xaa₄₆ is: His, Ser, Arg, Lys, L-cysteine, D-cysteine, homocysteine, penicillamine, NH₂, or is absent;
Xaa₄₇ is: His, Ser, Arg, Lys, L-cysteine, D-cysteine, homocysteine, penicillamine, NH₂, or is absent; and
Xaa₄₈ is: L-cysteine, D-cysteine, homocysteine, penicillamine, NH₂, or is absent;
   provided that if Xaa₄₂, Xaa₄₃, Xaa₄₄, Xaa₄₅, Xaa₄₆, or Xaa₄₇ is absent each amino acid downstream is absent and further provided that the GLP-1 peptide does not have the following C-terminal amino acid extension beginning at Xaa₃₆: Gly-Pro-Ser-Ser-Gly-Ala-Pro-Pro-Pro-Ser-NH₂.

The present invention further encompasses an extended GLP-1 peptide comprising the amino acid sequence of formula 5 (SEQ ID NO:5)

Formula 5

| | |
|---|---|
| Xaa₇-Xaa₈-Glu-Gly-Thr-Phe-Thr-Ser- | (SEQ ID NO:5) |
| Asp-Val-Ser-Ser-Tyr-Lys-Glu-Xaa₂₂- | |
| Gln-Ala-Xaa₂₅-Lys-Glu-Phe-Ile-Ala- | |
| Trp-Leu-Xaa₃₃-Lys-Gly-Gly-Pro-Xaa₃₈- | |
| Xaa₃₉-Xaa₄₀-Xaa₄₁-Xaa₄₂-Xaa₄₃-Xaa₄₄- | |
| Xaa₄₅-Xaa₄₆-Xaa₄₇-Xaa₄₈ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Gly, Val, Leu, Ile, Ser, or Thr;
Xaa₂₂ is: Gly, Glu, Asp, or Lys;
Xaa₂₅ is: Ala, Val, Ile, or Leu;
Xaa₃₃ is: Val or Ile;
Xaa₃₈ is: Ser, Pro, His, L-Cys, D-Cys, homocysteine, penicillamine, NH₂, or is absent;
Xaa₃₉ is: Ser, Arg, Thr, Trp, Lys, L-Cys, D-Cys, homocysteine, penicillamine, NH₂, or is absent;
Xaa₄₀ is: Ser, Gly, L-Cys, D-Cys, homocysteine, penicillamine, NH₂, or is absent;
Xaa₄₁ is: Ala, Asp, Arg, Glu, Lys, Gly, L-Cys, D-Cys, homocysteine, penicillamine, NH₂, or is absent;
Xaa₄₂ is: Pro, Ala, L-Cys, D-Cys, homocysteine, penicillamine, NH₂, or is absent;
Xaa₄₃ is: Pro, Ala, L-Cys, D-Cys, homocysteine, penicillamine, NH₂, or is absent;
Xaa₄₄ is: Pro, Ala, Arg, Lys, His, L-Cys, D-Cys, homocysteine, penicillamine, NH₂, or is absent;
Xaa₄₅ is: Ser, His, Pro, Lys, Arg, L-Cys, D-Cys, homocysteine, penicillamine, NH₂, or is absent;
Xaa₄₆ is: His, Ser, Arg, Lys, L-Cys, D-Cys, homocysteine, penicillamine, NH₂, or is absent;
Xaa₄₇ is: His, Ser, Arg, Lys, L-Cys, D-Cys, homocysteine, penicillamine, NH₂, or is absent; and
Xaa₄₈ is: L-Cys, D-Cys, homocysteine, penicillamine, NH₂, or is absent;
wherein said extended GLP-1 peptide contains a single L-Cys, D-Cys, homocysteine, or penicillamine which occurs at one of Xaa₃₈, Xaa₃₉, Xaa₄₀, Xaa₄₁, Xaa₄₂, Xaa₄₃, Xaa₄₄, Xaa₄₅, Xaa₄₆, Xaa₄₇, or Xaa₄₈, said GLP-1 is modified at said single L-Cys, D-Cys, homocysteine, or penicillamine; and
provided that if Xaa₃₈, Xaa₃₉, Xaa₄₀, Xaa₄₁, Xaa₄₂, Xaa₄₃, Xaa₄₄, Xaa₄₅, Xaa₄₆, or Xaa₄₇ is absent each amino acid downstream is absent.

In addition, the present invention encompasses an extended GLP-1 peptide comprising the amino acid sequence of formula 6 (SEQ ID NO:6)

Formula 6

| | |
|---|---|
| Xaa₇-Xaa₈-Glu-Gly-Thr-Xaa₁₂-Thr-Ser- | (SEQ ID NO:6) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Xaa₃₄-Gly- | |
| Xaa₃₆-Xaa₃₇-Xaa₃₈-Xaa₃₉-Xaa₄₀-Xaa₄₁- | |
| Xaa₄₂-Xaa₄₃-Xaa₄₄-Xaa₄₅-Xaa₄₆-Xaa₄₇- | |
| Xaa₄₈-Xaa₄₉-Xaa₅₀-Xaa₅₁ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, or Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val or Ile;
Xaa₃₄ is: Lys, Asp, Arg, or Glu;
Xaa₃₆ is: Gly, Pro, or Arg;
Xaa₃₇ is: Gly, Pro, or Ser;
Xaa₃₈ is: Ser, Pro, or His;
Xaa₃₉ is: Ser, Arg, Thr, Trp, or Lys;
Xaa₄₀ is: Ser or Gly;
Xaa₄₁ is: Ala, Asp, Arg, Glu, Lys, or Gly;
Xaa₄₂ is: Pro, Ala, L-Cys, D-Cys, homocysteine, penicillamine, NH₂, or is absent;
Xaa₄₃ is: Pro, Ala, L-Cys, D-Cys, homocysteine, penicillamine, NH₂, or is absent;
Xaa₄₄ is: Pro, Ala, Arg, Lys, His, NH₂, L-Cys, D-Cys, homocysteine, penicillamine, NH₂, or is absent;
Xaa₄₅ is: Ser, His, Pro, Lys, Arg, Gly, L-Cys, D-Cys, homocysteine, penicillamine, NH₂, or is absent;
Xaa₄₆ is: His, Ser, Arg, Lys, Pro, Gly, L-Cys, D-Cys, homocysteine, penicillamine, NH₂, or is absent;
Xaa₄₇ is: His, Ser, Arg, Lys, L-Cys, D-Cys, homocysteine, penicillamine, NH₂, or is absent;
Xaa₄₈ is: Gly, His, L-Cys, D-Cys, homocysteine, penicillamine, NH₂, or is absent;
Xaa₄₉ is: Pro, His, L-Cys, D-Cys, homocysteine, penicillamine, NH₂, or is absent;
Xaa₅₀ is: Ser, His, Ser-NH₂, His-NH₂, L-Cys, D-Cys, homocysteine, penicillamine, NH₂, or is absent; and
Xaa₅₁ is: L-Cys, D-Cys, homocysteine, penicillamine, NH₂, or is absent;
wherein said extended GLP-1 peptide contains a single L-Cys, D-Cys, homocysteine, or penicillamine which occurs at one of Xaa₄₂, Xaa₄₃, Xaa₄₄, Xaa₄₅, Xaa₄₆, Xaa₄₇, Xaa₄₈, Xaa_{49,} Xaa_{50,} or Xaa₅₁ said GLP-1 is modified at said single L-Cys, D-Cys, homocysteine, or penicillamine; and
provided that if Xaa₄₂, Xaa₄₃, Xaa₄₄, Xaa₄₅, Xaa₄₆, Xaa_{47,} Xaa_{48,} Xaa_{49,} or Xaa₅₀, is absent each amino acid downstream is absent and further provided that if Xaa₃₆ is Arg and Xaa₃₇ is Gly or Ser, the GLP-1 peptide does not have the following C-terminal amino acid extension beginning at Xaa₃₈: Ser-Ser-Gly-Ala-Pro-Pro-Pro-Ser-NH₂.

The present invention further encompasses an extended GLP-1 peptide comprising the amino acid sequence of 7 (SEQ ID NO:7)

Formula 7

| | |
|---|---|
| His-Ala-Glu-Gly-Thr-Phe-Thr-Ser-Asp- | (SEQ ID NO:7) |
| Val-Ser-Ser-Tyr-Leu-Glu-Gly-Gln-Ala- | |
| Ala-Lys-Glu-Phe-Ile-Ala-Trp-Leu-Val- | |
| Lys-Gly-Gly-Pro-Xaa₃₈-Xaa₃₉-Xaa₄₀- | |
| Xaa₄₁-Xaa₄₂-Xaa₄₃-Xaa₄₄-Xaa₄₅-Xaa₄₆- | |
| Xaa₄₇-Xaa₄₈-Xaa₄₉-Xaa₅₀-Xaa₅₁ | |

Wherein:
Xaa₃₈ is: Ser, Pro, orHis;
Xaa₃₉ is: Ser, Arg, Thr, Trp, or Lys;
Xaa₄₀ is: Ser or Gly;
Xaa₄₁ is: Ala, Asp, Arg, Glu, Lys, or Gly;
Xaa₄₂ is: Pro, Ala, L-Cys, D-Cys, homocysteine, penicillamine, NH₂, or is absent;
Xaa₄₃ is: Pro, Ala, L-Cys, D-Cys, homocysteine, penicillamine, NH₂, or is absent;
Xaa₄₄ is: Pro, Ala, Arg, Lys, His, NH₂, L-Cys, D-Cys, homocysteine, penicillamine, NH₂, or is absent;
Xaa₄₅ is: Ser, His, Pro, Lys, Arg, Gly, L-Cys, D-Cys, homocysteine, penicillamine, NH₂, or is absent;
Xaa₄₆ is: His, Ser, Arg, Lys, Pro, Gly, L-Cys, D-Cys, homocysteine, penicillamine, NH₂, or is absent;
Xaa₄₇ is: His, Ser, Arg, Lys, L-Cys, D-Cys, homocysteine, penicillamine, NH₂, or is absent;
Xaa₄₈ is: Gly, His, L-Cys, D-Cys, homocysteine, penicillamine, NH₂, or is absent;
Xaa₄₉ is: Pro, His, L-Cys, D-Cys, homocysteine, penicillamine, NH₂, or is absent;
Xaa₅₀ is: Ser, His, Ser-NH₂, His-NH₂, L-Cys, D-Cys, homocysteine, penicillamine, NH₂, or is absent; and
Xaa₅₁ is: L-Cys, D-Cys, homocysteine, penicillamine, NH₂, or is absent;
wherein said extended GLP-1 peptide contains a single L-Cys, D-Cys, homocysteine, or penicillamine which occurs at one of Xaa₄₂, Xaa₄₃, Xaa₄₄, Xaa₄₅, Xaa₄₆, Xaa₄₇, Xaa₄₈, Xaa_{49,} Xaa_{50,} or Xaa₅₁ said GLP-1 is modified at said single L-Cys, D-Cys, homocysteine, or penicillamine; and
provided that if Xaa₄₂, Xaa₄₃, Xaa₄₄, Xaa₄₅, Xaa₄₆, Xaa_{47,} Xaa_{48,} Xaa_{49,} or Xaa₅₀, is absent each amino acid downstream is absent.

The present invention further encompasses an extended GLP-1 peptide comprising the amino acid sequence of formula 10(SEQ ID NO:10)

Formula 10

| | |
|---|---|
| Xaa₇-Xaa₈-Glu-Gly-Thr-Xaa₁₂-Thr-Ser- | (SEQ ID NO:10) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Xaa₃₄-Gly- | |
| Xaa₃₆-Xaa₃₇-Xaa₃₈-Xaa₃₉-Xaa₄₀-Xaa₄₁- | |
| Xaa₄₂-Xaa₄₃-Xaa₄₄-Xaa₄₅-Xaa₄₆-Xaa₄₇- | |
| Xaa₄₈ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, or Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val or Ile;
Xaa₃₄ is: Lys, Asp, Arg, or Glu;
Xaa₃₆ is: Gly, Pro, or Arg;
Xaa₃₇ is: Gly, Pro, Ser, or Lys;
Xaa₃₈ is: Ser, Pro, His, Lys, NH₂;
Xaa₃₉ is: Ser, Arg, Thr, Trp, Lys, NH₂, or is absent;
Xaa₄₀ is: Ser, Gly, Lys, NH₂, or is absent;
Xaa₄₁ is: Ala, Asp, Arg, Glu, Lys, Gly, NH₂, or is absent;
Xaa₄₂ is: Pro, Ala, Lys, NH₂, or is absent;
Xaa₄₃ is: Pro, Ala, Lys, NH₂, or is absent;
Xaa₄₄ is: Pro, Ala, Arg, Lys, His, NH₂, or is absent;
Xaa₄₅ is: Ser, His, Pro, Lys, Arg, NH₂, or is absent;
Xaa₄₆ is: His, Ser, Arg, Lys, NH₂, or is absent;
Xaa₄₇ is: His, Ser, Arg, Lys, NH₂, or is absent; and
Xaa₄₈ is: Lys, NH₂, or is absent;
provided that if Xaa₃₉, Xaa₄₀, Xaa₄₁, Xaa₄₂, Xaa₄₃, Xaa₄₄, Xaa₄₅, Xaa₄₆, or Xaa₄₇ is absent each amino acid downstream is absent and further provided that the GLP-1 peptide does not have the following C-terminal amino acid extension beginning at Xaa₃₆: Gly-Pro-Ser-Ser-Gly-Ala-Pro-Pro-Pro-Ser-NH₂.

The present invention also encompasses an extended GLP-1 peptide comprising the amino acid sequence of formula 11 (SEQ ID NO:11)

Formula 11

| | |
|---|---|
| Xaa₇-Xaa₈-Glu-Gly-Thr-Phe-Thr-Ser- | (SEQ ID NO:11) |
| Asp-Xaa₁₆-Ser-Ser-Tyr-Lys-Glu-Xaa₂₂- | |
| Gln-Ala-Xaa₂₅-Lys-Glu-Phe-Ile-Ala- | |
| Trp-Leu-Xaa₃₃-Xaa₃₄-Gly-Xaa₃₆-Xaa₃₇- | |
| Xaa₃₈-Xaa₃₉-Xaa₄₀-Xaa₄₁-Xaa₄₂-Xaa₄₃- | |
| Xaa₄₄-Xaa₄₅-Xaa₄₆-Xaa₄₇-Xaa₄₈ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Gly, Val, Leu, Ile, Ser, or Thr;
Xaa₁₆ is: Val, Trp, Ilr, Leu, Phe, or Tyr;
Xaa₂₂ is: Gly, Glu, Asp, or Lys;
Xaa₂₅ is: Ala, Val, Ile, or Leu;
Xaa₃₃ is: Val or Ile;
Xaa₃₄ is: Lys, Asp, Arg, or Glu;
Xaa₃₆ is: Gly, Pro, or Arg;
Xaa₃₇ is: Gly, Pro, Ser, or Lys;
Xaa₃₈ is: Ser, Pro, His, Lys, NH₂, or is absent;
Xaa₃₉ is: Ser, Arg, Thr, Trp, Lys, NH₂, or is absent;
Xaa₄₀ is: Ser, Gly, Lys, NH₂, or is absent;
Xaa₄₁ is: Ala, Asp, Arg, Glu, Lys, Gly, NH₂, or is absent;
Xaa₄₂ is: Pro, Ala, Lys, NH₂, or is absent;
Xaa₄₃ is: Pro, Ala, Lys, NH₂, or is absent;
Xaa₄₄ is: Pro, Ala, Arg, Lys, His, NH₂, or is absent;
Xaa₄₅ is: Ser, His, Pro, Lys, Arg, Nh₂, or is absent;
Xaa₄₆ is: His, Ser, Arg, Lys, NH₂, or is absent
Xaa₄₇ is: His, Ser, Arg, Lys, NH₂, or is absent; and
Xaa₄₈ is: Lys, NH₂, or is absent;
   provided that if Xaa₃₉, Xaa₄₀, Xaa₄₁, Xaa₄₂, Xaa₄₃, Xaa₄₄, Xaa₄₅, Xaa₄₆, or Xaa₄₇ is absent each amino acid downstream is absent and further provided that the GLP-1 peptide does not have the following C-terminal amino acid extension beginning at Xaa₃₆: Gly-Pro-Ser-Ser-Gly-Ala-Pro-Pro-Pro-Ser-NH₂.

The present invention further encompasses an extended GLP-1 peptide comprising the amino acid sequence of formula 12 (SEQ ID NO:12)

Formula 12

| | |
|---|---|
| Xaa₇-Xaa₈-Glu-Gly-Thr-Phe-Thr-Ser- | (SEQ ID NO:12) |
| Asp-Val-Ser-Ser-Tyr-Lys-Glu-Xaa₂₂- | |
| Gln-Ala-Xaa₂₅-Lys-Glu-Phe-Ile-Ala- | |
| Trp-Leu-Xaa₃₃-Lys-Gly-Gly-Pro-Xaa₃₈- | |
| Xaa₃₉-Xaa₄₀-Xaa₄₁-Xaa₄₂-Xaa₄₃-Xaa₄₄- | |
| Xaa₄₅-Xaa₄₆-Xaa₄₇-Xaa₄₈ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Gly, Val, Leu, Ile, Ser, or Thr;
Xaa₂₂ is: Gly, Glu, Asp, or Lys;
Xaa₂₅ is: Ala, Val, Ile, or Leu;
Xaa₃₃ is: Val or Ile;
Xaa₃₈ is: Ser, Pro, His, Lys, NH₂, or is absent;
Xaa₃₉ is: Ser, Arg, Thr, Trp, Lys, NH₂, or is absent;
Xaa₄₀ is: Ser, Gly, Lys, NH₂,or is absent;
Xaa₄₁ is: Ala, Asp, Arg, Glu, Lys, Gly, NH₂, or is absent;
Xaa₄₂ is: Pro, Ala, Lys, NH₂, or is absent;
Xaa₄₃ is: Pro, Ala, Lys, NH₂, or is absent;
Xaa₄₄ is: Pro, Ala, Arg, Lys, His, NH₂, or is absent;
Xaa₄₅ is: Ser, His, Pro, Lys, Arg, NH₂, or is absent;
Xaa₄₆ is: His, Ser, Arg, Lys, NH₂, or is absent;
Xaa₄₇ is: His, Ser, Arg, Lys, NH₂, or is absent; and
Xaa₄₈ is: Lys, NH₂, or is absent;
   wherein said extended GLP-1 peptide is modified at a single Lys which occurs at one of Xaa₃₇, Xaa₃₈, Xaa₃₉, Xaa₄₀, Xaa₄₁, Xaa₄₂, Xaa₄₃, Xaa₄₄, Xaa₄₅, Xaa₄₆, Xaa₄₇, or Xaa₄₈; and
provided that if Xaa₃₈, Xaa₃₉, Xaa₄₀, Xaa₄₁, Xaa₄₂, Xaa₄₃, Xaa₄₄, Xaa₄₅, Xaa₄₆, or Xaa₄₇ is absent each amino acid downstream is absent.

In addition, the present invention encompasses an extended GLP-1 peptide comprising the amino acid sequence of formula 13 (SEQ ID NO:13)

Formula 13

| | |
|---|---|
| Xaa₇-Xaa₈-Glu-Gly-Thr-Xaa₁₂-Thr-Ser- | (SEQ ID NO:13) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Xaa₃₄-Gly- | |
| Xaa₃₆-Xaa₃₇-Xaa₃₈-Xaa₃₉-Xaa₄₀-Xaa₄₁- | |
| Xaa₄₂-Xaa₄₃-Xaa₄₄-Xaa₄₅-Xaa₄₆-Xaa₄₇- | |
| Xaa₄₈-Xaa₄₉-Xaa₅₀-Xaa₅₁ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, Phe;
Xaa₂₀ is: Leu, Phe, Tyr, or Trp;
Xaa₂₂ is: Gly, Glu, Asp, or Lys;
Xaa₂₅ is: Ala, Val, Ile, Leu;
Xaa₂₇ is: Glu, Ile, or Ala;
Xaa₃₀ is: Ala or Glu;
Xaa₃₃ is: Val or Ile;
Xaa₃₄ is: Lys, Asp, Arg, or Glu;
Xaa₃₆ is: Gly, Pro, Arg;
Xaa₃₇ is: Gly, Pro, or Ser;
Xaa₃₈ is: Ser, Pro, His;
Xaa₃₉ is: Ser, Arg, Thr, Trp, or Lys;
Xaa₄₀ is: Ser or Gly;
Xaa₄₁ is: Ala, Asp, Arg, Glu, Lys, or Gly;
Xaa₄₂ is: Pro, Ala, Lys, NH₂, or is absent;
Xaa₄₃ is: Pro, Ala, Lys, NH₂, or is absent;
Xaa₄₄ is: Pro, Ala, Arg, Lys, His, NH₂, or is absent;
Xaa₄₅ is: Ser, His, Pro, Lys, Arg, NH₂, or is absent;
Xaa₄₆ is: His, Ser, Arg, Lys, NH₂, or is absent;
Xaa₄₇ is: His, Ser, Arg, Lys, NH₂, or is absent; and
Xaa₄₈ is: Lys, NH₂, or is absent;
Xaa₄₉ is: Pro, His, Lys, NH₂, or is absent;
Xaa₅₀ is: Ser, His, Lys, NH₂, or is absent; and
Xaa₅₁ is: Lys, NH₂, or is absent;
   wherein said extended GLP-1 peptide is modified at a single Lys which occurs at one of Xaa₄₂, Xaa₄₃, Xaa₄₄, Xaa₄₅, Xaa₄₆, Xaa₄₇, Xaa₄₈, Xaa_{49,} Xaa_{50,} or Xaa₅₁; and provided that if Xaa₃₈, Xaa₃₉, Xaa40, Xaa₄₁, Xaa₄₂, Xaa₄₃, Xaa₄₄, Xaa₄₅, Xaa₄₆, Xaa_{47,} Xaa_{48,} Xaa_{49,} or Xaa₅₀, is absent each amino acid downstream is absent.

The present invention further encompasses an extended GLP-1 peptide comprising the amino acid sequence of formula 14 (SEQ ID NO:14)

Formula 14

| | |
|---|---|
| His-Ala-Glu-Gly-Thr-Phe-Thr-Ser-Asp- | (SEQ ID NO:14) |
| Val-Ser-Ser-Tyr-Leu-Glu-Gly-Gln-Ala- | |
| Ala-Lys-Glu-Phe-Ile-Ala-Trp-Leu-Val- | |
| Lys-Gly-Gly-Pro-Xaa₃₈-Xaa₃₉-Xaa₄₀- | |
| Xaa₄₁-Xaa₄₂-Xaa₄₃-Xaa₄₄-Xaa₄₅-Xaa₄₆- | |
| Xaa₄₇-Xaa₄₈-Xaa₄₉-Xaa₅₀-Xaa₅₁ | |

Wherein:
Xaa₃₈ is: Ser, Pro, or His;
Xaa₃₉ is: Ser, Arg, Thr, Trp, or Lys;
Xaa₄₀ is: Ser or Gly;
Xaa₄₁ is: Ala, Asp, Arg, Glu, Lys, or Gly;
Xaa₄₂ is: Pro, Ala, Lys, NH₂, or is absent;
Xaa₄₃ is: Pro, Ala, Lys, NH₂, or is absent;
Xaa₄₄ is: Pro, Ala, Arg, Lys, His, NH₂, or is absent;
Xaa₄₅ is: Ser, His, Pro, Lys, Arg, NH₂, or is absent;
Xaa₄₆ is: His, Ser, Arg, Lys, NH₂, or is absent;
Xaa₄₇ is: His, Ser, Arg, Lys, NH₂, or is absent; and
Xaa₄₈ is: Lys, NH₂, or is absent;
Xaa₄₉ is: Pro, His, Lys, NH₂, or is absent;
Xaa₅₀ is: Ser, His, Lys, NH₂, or is absent; and
Xaa₅₁ is: Lys, NH₂, or is absent;
   wherein said extended GLP-1 peptide is modified at a single Lys which occurs at one of Xaa₄₂, Xaa₄₃, Xaa₄₄, Xaa₄₅, Xaa₄₆, Xaa₄₇, Xaa₄₈, Xaa_{49,} Xaa_{50,} or Xaa₅₁; and provided that if Xaa₄₂, Xaa₄₃, Xaa₄₄, Xaa₄₅, Xaa₄₆, Xaa_{47,} Xaa_{48,} Xaa_{49,} or Xaa₅₀, is absent each amino acid downstream is absent.

The present invention encompasses the discovery that specific amino acids added to the C-terminus of a GLP-1 peptide provide specific structural features that protect the peptide from degradation by various proteases yet do not negatively impact the biological activity of the peptide. Further, many of the extended peptides disclosed herein are actually more potent than DPP-IV resistant GLP-1 analogs such as Val⁸-GLP-1(7-37)OH.

Reactive Groups

A GLP-1 compound of the present invention encompasses a GLP-1 peptide or an extended GLP-1 peptide that has been modified by attaching or coupling a reactive group to the GLP-1 peptide. A GLP-1 compound is thereby capable of covalently binding to a blood component through the reactive group. The reactive group typically will covalently bond with an amino group, a hydroxyl group, or a thiol group on a blood component, thereby covalently linking the GLP-1 peptide to the blood component. Preferably, the reactive group will react with a thiol group on a blood component. More preferably, the reactive group will react with a thiol group on blood serum albumin.

The reactive group may contain any of a number of chemically reactive entities that are capable of forming a covalent bond. Preferably, the reactive group will be capable of reacting with a thiol group on a blood component to form a disulfide bond. Reactive groups that are capable of forming disulfide bonds with thiol groups include those having an activated disulfide bond or an S-sulfonate. Reactive groups having an activated disulfide bond can be derived by coupling a GLP-1 peptide cysteine (or cysteine analog) with an activating group, such as 2,2′-dithiodipyridine (DTDP), 2,2′-dithiobis(5-Nitropyridine) (NPYS), 5,5′-dithiobis(2-nitrobenzoic acid) (Ellman's reagent), or 6,6′-dithiodinicotinic acid. Reactive groups containing an activated disulfide bond are herein referred to as activated disulfide bond groups.

In addition, an activated disulfide bond group can be derived by acylating a lysine side chain of a GLP-1 peptide with a mercapto-activated carboxylic acid. Alternatively, a lysine side chain of a GLP-1 peptide can be modified with either an activated disulfide bond group or an S-sulfonate in a step-wise manner. The lysine of the GLP-1 peptide could be first acylated with a protected thiol-containing carboxylic acid. The protected thiol of the acylated GLP-1 subsequently could be deprotected and modified to yield an activated disulfide bond or S-sulfonate, as described above for the modification of a cysteine thiol. A reactive group derived by modifying a lysine side chain with an activated disulfide bond group or S-sulfonate is respectively termed a modified lysine with an activated disulfide bond group or a modified lysine with a S-sulfonate.

Another preferred embodiment of the present invention is to utilize a reactive group that is capable of reacting with a thiol group on a blood component to form a thioether linkage. Preferably, such a reactive group will be derived by coupling a GLP-1 peptide with a chemically reactive entity from a maleimido-containing group, such as gamma-maleimide-butyrylamide (GMBA), maleimide-benzoyl-succinimide (MBS), gamma-maleimido-butyryloxy succinimide ester (GMBS), and maleimidopropionic acid (MPA). These and other maleimide containing groups are herein referred to as maleimido groups.

In an alternative embodiment of the present invention, the reactive group of a GLP-1 compound will be capable of covalently bonding to a primary amine on a blood component to form an amide bond. Preferably, such reactive groups will be derived by coupling a GLP-1 peptide with N-hydroxysuccinimide (NHS) or N-hydroxy-sulfosuccinimide (sulfo-NHS) to form an NHS or sulfo-NHS ester. These succinimide containing reactive groups are herein referred to as succinimidyl groups. These succinimidyl groups may potentially react with a-amine groups on the N-termini of blood component proteins, provided that such a-amine groups are accessible or available to the reactive group. Preferably, these succinimidyl groups will react with the ε-amine of lysine in blood component proteins, since the 6-amine of lysine is the only amino acid side chain that reacts significantly with NHS esters.

Specific Binding to Serum Albumin

The preferred GLP-1 compounds of the present invention contain reactive groups that are designed to covalently bond with thiol groups on blood components. Binding to thiol groups is preferred over binding to amino groups, because thiol groups are less abundant in vivo than are amino groups. Fewer blood components are thereby targeted through binding to thiol groups compared to binding to amino groups, resulting in greater specificity of binding. Accordingly, the preferred GLP-1 compounds will contain GLP-1 peptides modified with a maleimido group or more preferably, an S-sulfonate or an activated disulfide bond group.

While the GLP-1 compounds of the present invention may bind to any of several blood components that contain a free thiol group, the GLP-1 compounds preferably will covalently bond with the thiol group on serum albumin. Serum albumin is the most abundant blood protein, and contains a single thiol group, located at amino acid residue 34 in the protein (Cys³⁴), which is highly conserved among species. This amino acid has a relatively high level of reactivity compared to free thiols on other free-thiol containing proteins, which is primarily attributed to two of its properties. First, the serum albumin Cys³⁴ residue has a pK value of 5.5, whereas most protein cysteines typically have a pK value of about 8. This low pK value causes Cys³⁴ to predominantly reside in an ionized form under normal physiological conditions, which significantly increases the reactivity of Cys³⁴ compared to free-thiols on other proteins. Second, the structural location of Cys³⁴ in serum albumin protein also contributes to its reactivity. This amino acid resides in a crevice close to the surface of a loop of region V of the protein, such that Cys³⁴ is readily available for interaction. These properties of the Cys³⁴ residue of serum albumin render the protein highly reactive to GLP-1 compounds that contain reactive groups that specifically interact with thiols, such as an activated disulfide bond group, an S-sulfonate, or a maleimido group.

The binding of GLP-1 compounds to serum albumin not only provides specificity of binding, but also provides a reproducible formation of conjugates having a 1:1 binding of GLP-1 compound to serum albumin. The reproducibility of this 1:1 ratio is desirable for use of a GLP-1 compound as a therapeutic, since reproducible conjugates of GLP-1 compound and serum albumin will result upon administration of the GLP-1 compound. Furthermore, the reproducibility of 1:1 conjugates of GLP-1 compound and serum albumin is desirable for ex vivo or in vitro approaches to formations of conjugates. Conjugates can be formed ex vivo by combining GLP-1 compounds of the present invention with blood, allowing formation of the conjugates, and then administering the conjugate-containing blood to the host. GLP-1 compound-serum albumin conjugates can also be formed in vitro, by combining GLP-1 compound with recombinant serum albumin to form conjugates which can be administered. The reproducibility of 1:1 conjugates of GLP-1 compound and serum albumin provides for reproducible conjugates from ex vivo administration to administration or in vitro batch to batch preparation.

Peptide Synthesis

The GLP-1 peptides of the present invention can be prepared using recombinant DNA technology or by using standard methods of solid-phase peptide synthesis techniques. Peptide synthesizers are commercially available from, for example, Applied Biosystems in Foster City Calif. Reagents for solid phase synthesis are commercially available, for example, from Midwest Biotech (Fishers, Ind.). Solid phase peptide synthesizers can be used according to manufacturers instructions for blocking interfering groups, protecting the amino acid to be reacted, coupling, decoupling, and capping of unreacted amino acids.

Typically, an <-N-carbamoyl protected amino acid and the N-terminal amino acid on the growing peptide chain on a resin is coupled at room temperature in an inert solvent such as dimethylformamide, N-methylpyrrolidone or methylene chloride in the presence of coupling agents such as dicyclohexylcarbodiimide and 1-hydroxybenzotriazole and a base such as diisopropylethylamine. The <-N-carbamoyl protecting group is removed from the resulting peptide resin using a reagent such as trifluoroacetic acid or piperidine, and the coupling reaction repeated with the next desired N-protected amino acid to be added to the peptide chain. Suitable amine protecting groups are well known in the art and are described, for example, in Green and Wuts, “Protecting Groups in Organic Synthesis”, John Wiley and Sons, 1991, the entire teachings of which are incorporated by reference. Examples include t-butyloxycarbonyl (tBoc) and fluorenylmethoxycarbonyl (Fmoc).

After completion of synthesis, peptides are cleaved from the solid-phase support with simultaneous side-chain deprotection using standard hydrogen fluoride or trifluoroacetic acid cleavage protocols. Crude peptides are then further purified using Reversed-Phase Chromatography on Vydac C18 columns employing linear water-acetonitrile gradients with all solvents containing 0.1% trifluoroacetic acid (TFA). To remove acetonitrile, peptides are lyophilized from a solution containing 0.1% TFA, acetonitrile and water. Purity can be verified by analytical reversed phase chromatography. Identity of peptides can be verified by mass spectrometry. Peptides can be solubilized in aqueous buffers at neutral pH.

Modification of GLP-1 Peptides

A GLP-1 compound of the present invention is formed by modifying a GLP-1 peptide with a reactive group, wherein the reactive group is coupled to the GLP-1 peptide by a variety of methods, depending upon the nature of a given GLP-1 peptide to be modified and the reactive group. In some instances, a GLP-1 peptide may be first produced recombinantly or synthetically, and then subsequently coupled with the reactive group. In other instances, a GLP-1 peptide may be synthesized, and then coupled with a reactive group while the peptide is still attached to a resin support used in the synthesis. Specific methods for coupling various reactive groups to GLP-1 compounds are described herein.

A GLP-1 peptide that is modified at a cysteine or cysteine analog (such as D-cysteine, homocysteine, or penicillamine) with an activated disulfide bond group or S-sulfonate may be coupled to a reactive group as a free peptide. A free GLP-1 peptide is produced either recombinantly or synthetically, and is “free” in the sense that it is not attached to a resin or other components used in the production of the peptide. The free GLP-1 peptide will contain a single cysteine or cysteine analog. Modification at a cysteine or cysteine analog in a GLP-1 peptide with an activated disulfide bond group or S-sulfonate according to the present invention may be made with any GLP-1 peptide having an amino acid sequence that contains a cysteine or cysteine analog. Accordingly, the amino acid sequence of a GLP-1 peptide containing a single cysteine or cysteine analog may be selected among all of the GLP-1 peptides encompassed by formulas 1 (SEQ ID NO:1), 2 (SEQ ID NO:2), 3 (SEQ ID NO:3), 4 (SEQ ID NO:4), 5 (SEQ ID NO:5), 6 (SEQ ID NO:6), or 7 (SEQ ID NO:7) including those peptides that have been removed from the formulas by proviso. Preferably, the amino acid sequence of a GLP-1 peptide containing a single cysteine or cysteine analog will be selected among the GLP-1 peptides encompassed by formulas 1 (SEQ ID NO:1), 2 (SEQ ID NO:2), 3 (SEQ ID NO:3), 4 (SEQ ID NO:4), 5 (SEQ ID NO:5), 6 (SEQ ID NO:6), or 7 (SEQ ID NO:7). For the extended GLP-1 peptides of formulas 3, 4, and 5, the cysteine or cysteine analog may occur at any of amino acid positions 37 through 48. For the extended GLP-1 peptides of formulas 6 and 7 the cysteine or cysteine analog may occur at any of amino acid positions 37 through 51. Preferably, the cysteine will be the C-terminal amino acid of the extended GLP-1 peptide.

An activated disulfide bond group is coupled to a GLP-1 peptide cysteine or cysteine analog through a method for the preferential formation of intermolecular disulfide bonds based on a selective thiol activation scheme. Methods based on the selective activation of one thiol with an activating group followed by a reaction with a second free thiol to form asymmetric disulfide bonds selectively between proteins or peptides have been described to alleviate the problem of reduced yields due to symmetric disulfide bond formation (D. Andreu, F. Albericio, N. A. Sole, M. C. Munson, M. Ferrer, and G. Barany, in “Methods in Molecular Biology” (M. W. Pennington and B. M. Dunn, eds.), Vol. 35, p. 91. Humana Press, Totowa, N.J., 1994). Preferably, such activating groups are those based on the pyridine-sulfenyl group (M. S. Bernatowicz, R. Matsueda, and G. R. Matsueda, Int. J. Pept. Protein Res. 28, 107(1986)). Preferably, 2,2′-dithiodipyridine (DTDP, J. Carlsson, H. Drevin, and R. Axen, Biochem. J. 173, 723(1978); L. H. Kondejewski, J. A. Kralovec, A. H. Blair, and T. Ghose, Biocojugate Chem. 5, 602(1994) or 2,2′-dithiobis(5-Nitropyridine) (NPYS, J Org. Chem. 56, 6477(1991)) is employed. In addition, 5,5′-dithiobis(2-nitrobenzoic acid) (Ellman's reagent) or 6,6′-dithiodinicotinic acid may be used as activating groups

In accordance with these methods, a disulfide bond activating group is first reacted with a GLP-1 peptide containing a cysteine or cysteine analog under conditions of excess activating group. These conditions highly favor the formation of the GLP-1 compound containing a GLP-1 peptide coupled with an activated disulfide group, with essentially no production of disulfide-bonded GLP-1 homodimers. Following the coupling reaction, the resulting GLP-1 compound is purified, such as by reversed phase-HPLC. A reaction with a second free thiol occurs when the GLP-1 compound is reacted with a blood component, preferably serum albumin, to form a conjugate between the GLP-1 compound and serum albumin. Formation of a GLP-1 compound containing an activated disulfide group coupled to a cysteine in a GLP-1 peptide is described below in Example 1.

A GLP-1 peptide cysteine or cysteine analog is converted to having an S-sulfonate through a sulfitolysis reaction scheme. In this scheme, a GLP-1 peptide is first synthesized either synthetically or recombinantly. A sulfitolysis reaction is then used to attach a S-sulfonate to the GLP-1 peptide through its cysteine or cysteine analog thiol. Following the sulfitolysis reaction, the GLP-1 compound is purified, such as by gradient column chromatography. The GLP-1 compound S-sulfonate is then used to form a conjugate between the GLP-1 compound and a blood component, preferably serum albumin. Preparation of a GLP-1 peptide containing a S-sulfonate attached to a cysteine is demonstrated in Example 2.

A GLP-1 peptide which is modified at a lysine with an activated disulfide bond group or a S-sulfonate is produced by attaching a reactive group to a chemically synthesized peptide. Modification at a lysine in a GLP-1 peptide with an activated disulfide bond group or S-sulfonate according to the present invention may be made with any GLP-1 peptide having an amino acid sequence that contains a lysine. Accordingly, the amino acid sequence of a GLP-1 peptide containing a lysine analog may be selected among all of the GLP-1 peptides encompassed by formulas 8 (SEQ ID NO:8), 9 (SEQ ID NO:9), 10 (SEQ ID NO:10), 11 (SEQ ID NO:11), 12 (SEQ ID NO:12), 13 (SEQ ID NO:13), and 14 (SEQ ID NO:14), including those peptides that have been removed from the formulas by proviso. Preferably, the amino acid sequence of a GLP-1 peptide containing a lysine to be modified will be selected among the GLP-1 peptides encompassed by formulas 8 (SEQ ID NO:8), 9 (SEQ ID NO:9), 10 (SEQ ID NO:10), 11 (SEQ ID NO:11), 12 (SEQ ID NO:12), 13 (SEQ ID NO:13), and 14 (SEQ ID NO:14). For the GLP-1 peptides of formulas 8 and 9, the peptide is modified at the lysine which occurs at amino acid position 37. For the extended GLP-1 peptides of formulas 10, 11 and 12, the peptide is modified at any of the lysines which may occur at any of amino acid positions 37 through 48, with only one lysine being modified within a given peptide. For the extended GLP-1 peptides of formulas 13 and 14, the peptide is modified at any of the lysines which may occur at any of amino acid positions 37 through 51, with only one lysine being modified within a given peptide. Preferably, the modified lysine will be the C-terminal amino acid of the extended GLP-1 peptide.

To produce a GLP-1 peptide having a modified lysine with an activated disulfide bond group, a GLP-1 peptide is first chemically synthesized such that the lysine to be modified has an orthogonal protecting group. For example, the majority of a GLP-1 peptide may be synthesized on mbha resin using t-butyloxycarbonyl (tBoc) protected amino acids, with the following side chain protecting groups: His(Bom), Glu(CHXL), Asp(CHXL), Ser(OBzl), Thr(OBzl), Tyr(Br-Z), Lys(Cl-Z), Trp(CHO), and Arg(Tos). The side chain of the lysine to be modified in this instance may be orthogonally protected with fluorenylmethoxycarbonyl (Fmoc). At the completion of the polypeptide chain synthesis, the peptidyl resin may be treated to selectively remove the orthogonal protecting group from the lysine to be modified (such as the orthogonal Fmoc above). The deprotected lysine side chain may then be acylated with a mercapto-activated carboxylic acid to render a modified GLP-1 peptide that will react with a thiol-containing blood component. The remainder of the GLP-1 compound is next deprotected, and then purified, such as by reverse phase column chromatography.

According to this method of coupling a lysine side chain amino group of a GLP-1 peptide with a mercapto-activated carboxylic acid, the lysine side chain could be acylated with any structure derived from the following general structure: 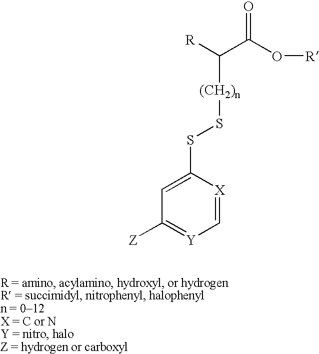
Preparation of a GLP-1 compound containing an activated disulfide bond group attached to a lysine in a GLP-1 peptide is provided below in Example 3.

A GLP-1 peptide having a modified lysine with an activated disulfide bond group or an S-sulfonate may be produced stepwise by first acylating a lysine side chain amino group with a protected thiol-containing carboxylic acid. After deprotection of this thiol, the peptide can then be modified with an activated disulfide bond group or an S-sulfonate. In this scenario, a GLP-1 peptide is first chemically synthesized such that the lysine to be modified has an orthogonal protecting group, as described above in the preceding paragraph. Following deprotection of the lysine side chain of interest, the deprotected lysine side chain may then be acylated with a structure derived from the following general structure:

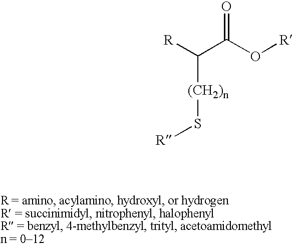

Following acylation of the lysine side chain with this structure, the thiol-containing structure is deprotected and the thiol is then coupled with an activating disulfide bond group or a S-sulfonate. The coupling of the activating disulfide bond group is carried out as described above in the method for producing a GLP-1 peptide that is modified at a cysteine with an activated disulfide bond group. Likewise, the attachment of the S-sulfonate is carried out as described above in the method for producing a GLP-1 peptide that is modified at a cysteine with a S-sulfonate.

In addition to GLP-1 peptides, the above described modifications to cysteine and lysine side chains may be made to Exendin 3 and Exendin 4 peptides and analogs thereof containing various natural or non-natural amino acid substitutions, deletions, and/or additions. Exendin 3 and exendin 4 are 39 amino acid peptides (differing at residues 2 and 3) which are approximately 53% homologous to GLP-1 and have insulinotropic activity. Exendin 3 has the sequence: HSDGTFTSDLSKQMEEEAVRLFIEWLKNGG PSSGAPPPS (SEQ ID NO:23) and exendin 4 has the sequence: HGEGTFTSDLSKQMEEEAVRLFIEWLKNGG PSSGAPPPS (SEQ ID NO:24).

A GLP-1 peptide modified with a maleimido group can be produced by attaching the reactive group to the carboxylic acid at the C-terminus of a chemically synthesized GLP-1 peptide. Preferably, the amino acid sequence of a GLP-1 peptide containing a maleimido group will be selected among the GLP-1 peptides encompassed by formulas 15 (SEQ ID NO:15), 10 (SEQ ID NO:10), and 13 (SEQ ID NO:13). Alternatively, a GLP-1 peptide may be modified with a maleimido group at a free amino group, such as on a lysine side chain. In this case of modifying a lysine, the amino acid sequence of a GLP-1 peptide of formula 15 (SEQ ID NO:15) will contain a lysine at amino acid position 37, and that lysine will be modified with a maleimido group. For modification of a lysine in an extended GLP-1 peptide of formula 10 (SEQ ID NO:10), the peptide is modified at any of the lysines which may occur at any of amino acid positions 37 through 48, with only one lysine being modified for a given peptide. For modification of a lysine in an extended GLP-1 peptide of formula 13 (SEQ ID NO:13), the peptide is modified at any of the lysines which may occur at any of amino acid positions 37 through 51, with only one lysine being modified for a given peptide. Preferably, the modified lysine will be the C-terminal amino acid of the extended GLP-1 peptide.

To synthesize a GLP-1 peptide that is modified at its C-terminal carboxylic acid with a maleimido group, the GLP-1 peptide is first synthesized as a fully protected peptide attached to a resin. The GLP-1 peptide is then cleaved from the resin, and the free C-terminus is reacted with a maleimido group, such as maleimidopropionic acid in the presence of ethylenediamine, as described in U.S. Pat. No. 6,329,336. The peptide protecting groups are then cleaved, and the GLP-1 compound is purified, such as by extraction, precipitation, and HPLC.

A GLP-1 peptide that is modified with a maleimido group at a free amino, such as on a lysine side chain, may be synthesized from a GLP-1 peptide containing a free amino group and a free carboxylic acid. In this case, a GLP-1 peptide is first chemically synthesized on a resin, with a lysine of interest having an orthogonal protecting group. The orthogonal protecting group is then selectively removed, and the peptide is cleaved from the resin. The peptide is then reacted to couple a maleimido group to the free amino group on the peptide. This reaction can be performed with N-[-maleimidobutyryloxy]succinimide ester (GMBS) and triethylamine in DMF. The succinimide ester group will react with the free amino and the modified GLP-1 peptide is subsequently purified from the reaction mixture by crystallization or by chromatography on silica or by HPLC.

A GLP-1 peptide that is modified with a maleimido group at a free amino, such as on a lysine side chain, also may be synthesized from an GLP-1 peptide containing a free amino group and no free carboxylic groups. For example, a GLP-1 peptide is first chemically synthesized on a resin with an orthogonal protecting group on a lysine of interest. After removal of the orthogonal protecting group, the free amino on the lysine side chain is reacted with a maleimido group, such as maleimidopropionic acid (MPA). The MPA can be coupled to the free amine to produce a maleimide derivative through reaction of the free amine with the carboxylic group of MPA using HBTU/HOBt/DIEA activation in DMF. The modified peptide is then cleaved from the resin, and purified, such as by precipitation followed by HPLC.

A GLP-1 peptide modified with a succinimidyl group may be produced by attaching the reactive group to the carboxylic acid at the C-terminus of a chemically synthesized GLP-1 peptide. Preferably, the amino acid sequence of a GLP-1 peptide containing a succinimidyl group will be selected from among the GLP-1 peptides encompassed by formulas 15 (SEQ ID NO:15), 10 (SEQ ID NO:10), and 13 (SEQ ID NO:13).

To produce a GLP-1 peptide that is modified with a succinimidyl group through attaching the reactive group to the carboxylic acid at the C-terminus, a fullly protected peptide is first synthesized on a resin. Preferably, the peptide contains no amino or thiol groups. In the instance where one or more amino or thiol groups is present in the peptide, it is necessary to protect these groups both prior to the succinimidyl attachment and after attachment, to prevent formation of covalently bonded peptide dimers. After peptide synthesis, the protected GLP-1 peptide is cleaved from the resin, and the succinimidyl is attached to the carboxyl group of the C-terminus. Specifically, the peptide is reacted with N-hydroxysuccinimide in anhydrous CH₂Cl₂ and EDC, and the product is purified by chromatography or recrystallized from the appropriate solvent system to yield the produced GLP-1 compound.

A GLP-1 peptide modified with a succinimidyl group may also be produced by attaching the reactive group to a GLP-1 peptide that contains a free amino in the absence of a free carboxylic acid. Preferably, the amino acid sequence of a GLP-1 peptide containing a succinimidyl group attached to an amino group will be selected from among the GLP-1 peptides encompassed by formulas 15 (SEQ ID NO:15), 10 (SEQ ID NO:10), 13 (SEQ ID NO:13). For the GLP-1 peptides of formula 15 (SEQ ID NO:15), the peptide contains a lysine at amino acid position 37 and is modified at that lysine. For an extended GLP-1 peptide of formula 10 (SEQ ID NO:10), the peptide is modified at any of the lysines which may occur at any of amino acid positions 37 through 48, with only one of these lysines being modified for a given peptide. For an extended GLP-1 peptide of formula 13 (SEQ ID NO:13), the peptide is modified at any of the lysines which may occur at any of amino acid positions 37 through 51, with only one of these lysines being modified for a given peptide. Preferably, the modified lysine will be the C-terminal amino acid of the extended GLP-1 peptide.

To produce a GLP-1 peptide that is modified with a succinimidyl group at an amino of a lysine side chain from a GLP-1 peptide that does not contain a free carboxylic acid, the peptide is first synthesized on a resin with appropriate protection groups. In particular, an orthogonal protection group is used on the lysine side chain of interest. Following synthesis of the peptide and removal of the orthogonal protection group, any of a number of succinimidyl groups may be used to modify the peptide. For example, addition of ethylene glycol-bis(succinimydylsuccinate) (EGS) and triethylamine dissolved in DMF to the free amino containing peptide (at a ratio of 10:1 in favor of EGS) will produce a modified GLP-1 peptide. The modified GLP-1 peptide is then cleaved from the resin and purified, such as by chromatography on silica or HPLC.

A GLP-1 peptide modified with a succinimidyl group alternatively may also be produced by attaching the reactive group to a GLP-1 peptide that contains a free thiol in the absence of a free carboxylic acid. Preferably, the amino acid sequence of a GLP-1 peptide containing a succinimidyl group attached to a thiol group will be selected from among the GLP-1 peptides encompassed by formulas 1 (SEQ ID NO:1), 3 (SEQ ID NO:3), and 6 (SEQ ID NO:6). For a GLP-1 peptide of formula 1 (SEQ ID NO:1), the peptide contains a cysteine or cysteine analog at amino acid position 37 and is modified at that position. For an extended GLP-1 peptide of formula 3 (SEQ ID NO:3), the peptide is modified at any of the cysteines or cysteine analogs which may occur at any of amino acid positions 37 through 48, with only one of these cysteines being modified for a given peptide. For an extended GLP-1 peptide of formula 6 (SEQ ID NO:6), the peptide is modified at any of the cysteines or cysteine analogs which may occur at any of amino acid positions 37 through 51, with only one of these lysines being modified for a given peptide. Preferably, the modified cysteine or cysteine analog will be the C-terminal amino acid of the extended GLP-1 peptide.

To produce a GLP-1 peptide that is modified with a succinimidyl group at a cysteine thiol from a GLP-1 peptide that does not contain a free carboxylic acid, the peptide is first synthesized on a resin with appropriate protection groups. An orthogonal protection group is used on the cysteine or cysteine analog side chain of interest, to enable its specific deprotection. After the peptide is synthesized and the orthogonal protection group is removed, any of a number of succinimidyl groups may be used to modify the free thiol on the peptide. For example, N-[gamma-maleimidobutyryloxy]succinimide ester (GMBS) and triethylamine in DMF can be used. The modified GLP-1 peptide is then cleaved from the resin and purified, such as by chromatography on silica or HPLC.

Each of the specific methods for coupling reactive groups to GLP-1 peptides provided above describe coupling a reactive group directly to a GLP-1 peptide. In addition to direct coupling, a reactive group may be attached to a GLP-1 peptide through a linking group, which effectively provides a spacer between the GLP-1 peptide and the reactive group. Suitable linking groups may comprise one or more alkyl groups such as methyl, ethyl, propyl, butyl, etc. groups, alkoxy groups, alkenyl groups, alkynyl groups or amino group substituted by alkyl groups, cycloalkyl groups, polycyclic groups, aryl groups, polyaryl groups, substituted aryl groups, heterocyclic groups, and substituted heterocyclic groups. Linking groups may also comprise poly ethoxy aminoacids such as (2-amino) ethoxy acetic acid or [2-(2-amino)ethoxy)]ethoxy acetic acid.

GLP-1 Compound Properties

For the purposes of the present invention an in vitro GLP-1 receptor signaling assay is used to determine whether a particular GLP-1 compound will exhibit insulinotropic activity in vivo. GLP-1 compounds encompassed by the present invention have an in vitro potency that is not less than 1/10 the in vitro potency of the DPP-IV resistant GLP-1 analog known as Val⁸-GLP-1(7-37)OH. More preferably, the extended GLP-1 peptides of the present invention are as potent or more potent than Val⁸-GLP-1(7-37)OH.

“In vitro potency” as used herein is the measure of the ability of a peptide to activate the GLP-1 receptor in a cell-based assay. In vitro potency is expressed as the “EC₅₀” which is the effective concentration of compound that results in 50% activity in a single dose-response experiment. For the purposes of the present invention, in vitro potency is determined using a fluorescence assay that employs HEK-293 Aurora CRE-BLAM cells that stably express the human GLP-1 receptor. These HEK-293 cells have stably integrated a DNA vector having a cAMP response element (CRE) driving expression of the β-lactamase (BLAM) gene. The interaction of a GLP-1 agonist with the receptor initiates a signal that results in activation of the cAMP response element and subsequent expression of β-lactamase. The β-lactamase CCF2/AM substrate that emits fluorescence when it is cleaved by β-lactamase (Aurora Biosciences Corp.) can then be added to cells that have been exposed to a specific amount of GLP-1 agonist to provide a measure of GLP-1 agonist potency. The assay is further described in Zlokarnik et al. (1998) Science 279:84-88 (See also Example 4). Relative in vitro potency values are established by running Val⁸-GLP-1(7-37)OH as a control and assigning the control a reference value of 1.

The GLP-1 compounds of the present invention provide for increased half-lives of the GLP-1 peptides contained within the compounds through conjugation of the GLP-1 peptides to a blood component, preferably serum albumin. Without being limited to any particular theories, conjugation of the GLP-1 peptide to serum albumin is anticipated to reduce the peptide's susceptibility to protease degradation. A measure of protease insensitivity is determined by exposing a GLP-1 compound-serum albumin conjugate and Val⁸-GLP-1 (7-37)OH to α-chymotrypsin and then plotting the progress of the enzymatic reaction, as described in Example 5.

GLP-1 Compound Administration and Therapeutic Use

The GLP-1 compounds of the present invention are suited for parenteral administration. Parenteral administration can include, for example, systemic administration, such as by intramuscular, intravenous, subcutaneous, or intraperitoneal injection. The GLP-1 compounds can be administered to the subject in conjunction with an acceptable pharmaceutical carrier, diluent or excipient as part of a pharmaceutical composition for treating various diseases and conditions discussed herein. The pharmaceutical composition can be a solution or a suspension. Suitable pharmaceutical carriers may contain inert ingredients which do not interact with the peptide or peptide derivative. Standard pharmaceutical formulation techniques may be employed such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Some examples of suitable excipients include lactose, dextrose, sucrose, trehalose, sorbitol, and mannitol.

The GLP-1 compounds described herein can be used to treat subjects with a wide variety of diseases and conditions. The GLP-1 compounds encompassed by the present invention exert their biological effects by acting at a receptor referred to as the “GLP-1 receptor” (see U.S. Pat. No. 5,670,360 to Thorrens). Subjects with diseases and/or conditions that respond favorably-to GLP-1 receptor stimulation or to the administration of GLP-1 compounds can therefore be treated. These subjects are said to “be in need of treatment with GLP-1 compounds” or “in need of GLP-1 receptor stimulation”.

Included are subjects with non-insulin dependent diabetes, insulin dependent diabetes, stress-induced hyperglycemia, stroke (see WO 00/16797 by Efendic), myocardial infarction (see WO 98/08531 by Efendic), obesity (see WO 98/19698 by Efendic), catabolic changes after surgery (see U.S. Pat. No. 6,006,753 to Efendic), functional dyspepsia and irritable bowel syndrome (see WO 99/64060 by Efendic). Also included are subjects requiring prophylactic treatment with a GLP-1 peptide, e.g., subjects at risk for developing non-insulin dependent diabetes (see WO 00/07617). Additional subjects include those with impaired glucose tolerance or impaired fasting glucose, subjects whose body weight is about 25% above normal body weight for the subject's height and body build, subjects with a partial pancreatectomy, subjects having one or more parents with non-insulin dependent diabetes, subjects who have had gestational diabetes and subjects who have had acute or chronic pancreatitis and are at risk for developing non-insulin dependent diabetes.

The GLP-1 compounds of the present invention can be used to normalize blood glucose levels, prevent pancreatic β-cell deterioration, induce β-cell proliferation, stimulate insulin gene transcription, up-regulate IDX-1/PDX-1 or other growth factors, improve β-cell function, activate dormant β-cells, differentiate cells into β-cells, stimulate β-cell replication, inhibit β-cell apoptosis, regulate body weight, and induce weight loss.

An “effective amount” of a GLP-1 compound is the quantity which results in a desired therapeutic and/or prophylactic effect without causing unacceptable side-effects when administered to a subject in need of GLP-1 receptor stimulation. A “desired therapeutic effect” includes one or more of the following: 1) an amelioration of the symptom(s) associated with the disease or condition; 2) a delay in the onset of symptoms associated with the disease or condition; 3) increased longevity compared with the absence of the treatment; and 4) greater quality of life compared with the absence of the treatment. For example, an “effective amount” of a GLP-1 compound for the treatment of type 2 diabetes is the quantity that would result in greater control of blood glucose concentration than in the absence of treatment, thereby resulting in a delay in the onset of diabetic complications such as retinopathy, neuropathy or kidney disease. An “effective amount” of a GLP-1 compound for the prevention of diabetes is the quantity that would delay, compared with the absence of treatment, the onset of elevated blood glucose levels that require treatment with drugs such as sulfonylureas, thiazolidinediones, insulin and/or bisguanidines.

A typical dose range for the GLP-1 compounds of the present invention will range from about 1 μg to about 100 mg per day. Preferably, the dose range is about 5 μg to about 1 mg per day. Even more preferably the dose is about 10 μg to about 100 μg per day.

A “subject” is a mammal, preferably a human, but can also be an animal, e.g., companion animals (e.g., dogs, cats, and the like), farm animals (e.g., cows, sheep, pigs, horses, and the like) and laboratory animals (e.g., rats, mice, guinea pigs, and the like).

The invention is illustrated by the following examples, which are not intended to be limiting in any way.

### EXAMPLES

### Example 1

### Preparation of a GLP-1 Compound Containing an Activated Disulfide Group Coupled to a Cysteine Thiol in a GLP-1 Peptide and Subsequent Conjugation to Human Serum Albumin

A GLP-1 compound containing an activated disulfide group coupled to the extended GLP-1 peptide HVEGTFTSDVSSYLEEQAAKEFIAWLIKGGPSSGAPPPC (SEQ ID NO:17) was synthesized and then conjugated to human serum albumin (HSA) according to the following reaction scheme:

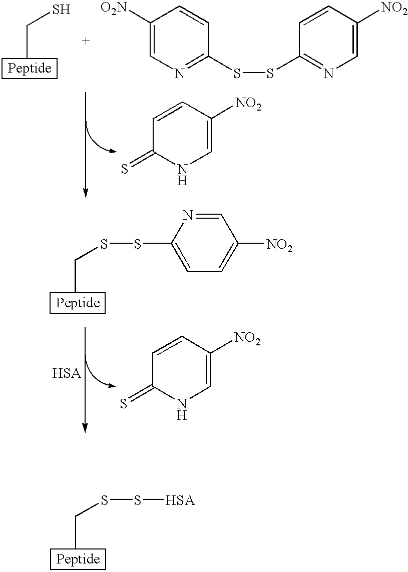

Specifically, the GLP-1 compound was formed by the following reaction scheme: 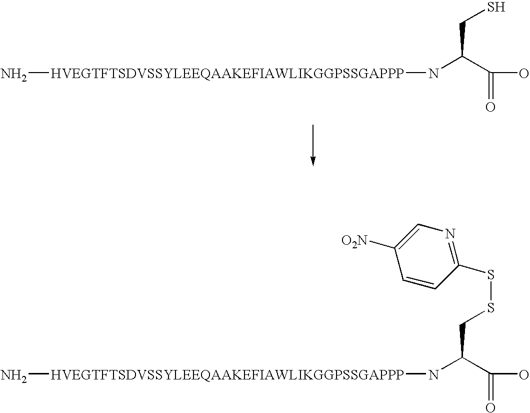
The cysteine-containing GLP-1 peptide was dissolved in methanol (DMF also may be used) at a concentration of 1 mg/mL and 3-fold molar excess of NPYS (DTP or Ellman's reagent alternatively may be used) was added. The solution was incubated at room temperature for 30 minutes. On completion of the reaction (which was confirmed by LC-MS), organic solvent was removed and the derivatized NPYS-peptide (SEQ ID NO:18) was isolated by RP-HPLC. The lyophilized NPYS-peptide and the underivatized human serum albumin were dissolved separately in degassed 50 mM sodium phosphate buffer, pH 7, containing 1 mM EDTA at a concentration of 1 mg/mL. The NPYS-peptide was slowly titrated with the human serum albumin solution. The optimal ratio for peptide/human serum albumin is 1 to 0.98. The progress of the reaction is monitored by RP-HPLC and the identity of the products confirmed by mass spectrometry (MALDI).

### Example 2

### Preparation of GLP-1 Compound Containing a S-Sulfonate Attached to a Cysteine Thiol in a GLP-1 Peptide and Subsequent Conjugation to Human Serum Albumin

0.5 gm of mbha-resin (Advanced ChemTech) was placed in a standard 60 ml reaction vessel and the GLP-1 extended peptide sequence below was entered and run on an ABI 430A peptide synthesizer using Boc amino acids and symmetric anhydride and HOBt activated double couplings.

(SEQ ID NO:19)

Boc-HVEGTFTSDVSSYLEEQAAKEFIAWLIKGRGC-mbha

Side chain protecting groups used include: His(Bom), Glu(CHXL), Asp(CHXL), Arg(Tos), Ser(OBzl), Thr(OBzl), Tyr(Br-Z), Lys(Cl-Z), Trp(CHO), and Cys(pMeBzl). The completed peptidyl resin was treated with 20% piperidine in DMF to deformylate the Trp, then washed with DMF, with DCM, transferred to a 200 ml Teflon HF reaction vessel and dried in vacuo to give 2.26 gm. 2 ml m-cresol, 0.5 gm p-thiocresol, and a magnetic stir bar were added. The vessel was attached to the HF apparatus (Peninsula Labs), cooled to −78° C., evacuated, and 20 ml liquid hydrogen fluoride was condensed in. The reaction was stirred 1 hour in an ice bath then the HF was distilled off. The residue was suspended in 180 ml ethyl ether and the resin/peptide solids were filtered and washed with ether 3-4 times.

The peptide was extracted into 100 ml of freshly prepared sulfitolysis solution (6 M guanidine/0.1 M tris, 35 mg sodium sulfite and 25 mg of sodium tetrathionate per 150 ml water (pH 8.6). The sulfitolysis reaction mixture was stirred at room temperature for 1 hour and then diluted with 100 ml of 10% aqueous acetic acid. This solution was loaded onto a 2.2×25 cm TosoHaas CG-71 column. A gradient of 20%-100% B (A=0.1% TFA, B=0.1% TFA/50% acetonitrile) at a flow rate of 4 ml/min was run over 15 hours using a Pharmacia FPLC pumps/controller. Five minute fractions were collected while monitoring the UV absorbance at 214 nm (2.0A). Based on the UV trace, the following fractions were combined and lyophilized into the following pools: A (fractions 83-90), B (fractions 93-100, 64.2 mg), C (fractions 101-112, 134.6 mg), D (fractions 113-124, 96.7 mg), and E (fractions 125-137, 78.1 mg). HPLC analysis showed that pools B through E each contained one major co-eluting peak having an approximate purity of 90%. Mass spectral analysis of pool B showed ions that were consistent with the theoretical molecular weight of 3652.02 for

| | |
|---|---|
| HVEGTFTSDVSSYLEEQAAKEFIA | (GLP-1 V8E22I33C38(SSO3)- |
| WLIKGRGC(SSO3)-amide. | amide; SEQ ID NO:20) |

The GLP-1 V8E22I33C38(SSO3)-amide compound (SEQ ID NO:20) was conjugated to human serum albumin. Specifically, 1.3 mg (0.35 μmole) GLP V8E22I33C38(SSO3)-amide (from pool D) and 22 mg (0.33 μmole) human serum albumin (Calbiochem) were dissolved in 1 ml PBS (phosphate buffered saline) containing 10 mg/ml EDTA (ethylene diamine tetraacetic acid). The reaction (at pH 7.5) was mixed and allowed to set at room temperature for approximately 50 hours. The reaction mixture was loaded onto a Pharmacia mono Q (HR16/10) ion exchange column equilibrated in buffer A (0.025M tris (pH 8.5), 30% acetonitrile). Using Pharmacia FPLC pumps, a gradient of 20% to 100% buffer B (buffer B=0.025M tris (pH8.5), 30% acetonitrile, 0.5M NaCl) was run over 160 minutes at a flow rate of 2 ml/minute and 2 minute fractions were collected while monitoring the UV absorbance at 214 nm (1.0A). The fractions associated with a large peak were combined into three pools and lyophilized: A (fractions 30-34), B (fractions 38-44), and C (fractions 45-54). Pools A and B were lyophilized to yield 0.9 mg A and 0.6 mg B. The product from A was re-dissolved in 2 ml water and was loaded onto a 1.0×25 cm Zorbax C8 column for desalting. A gradient of 20% to 80% B was run at 1 ml/min (A=0.1% TFA; B=0.1% TFA/90% acetonitrile), and the UV absorbance was monitored at 214 nm (1.0A) while collecting 2 ml/min fractions. MALDI mass spectral analysis of pool A showed an approximate ratio of HSA to GLP-C38-SS-HSA conjugate of 60:40.

### Example 3

### Preparation of GLP-1 Compound Containing an Activated Disulfide Group Coupled to a Lysine in a GLP-1 Peptide and Subsequent Conjugation to Human Serum Albumin

0.69 gm (0.43 mmole) mbha-resin (4-methyl benzhydrylamine) (Advanced ChemTech) was placed in a 60 ml reaction vessel and the extended GLP-1 sequence below was entered and run on an Applied Biosystems 430A peptide synthesizer using either symmetric anhydride or 1-hydroxybenzotriazole active ester double couplings with Boc protected amino acids.

(SEQ ID NO:21)

Boc-HVEGTFTSDVSSYLEEQAAKEFIAWLIKGRGK-mbha

Side chain protecting groups used were His(Bom), Glu(CHXL), Asp(CHXL), Ser(OBzl), Thr(OBzl), Tyr(Br-Z), Lys(Cl-Z), Trp(CHO), and Arg(Tos). The side chain of the C-terminal Lys was protected with an FMOC group. At the completion of the peptide chain assembly, the peptidyl resin was treated with 20% piperidine in dimethylformamide to selectively remove the lysine-FMOC group.

After washing the resin, it was treated with 100 mg (0.32 mmole) N-succinimidyl-3-(2-pyridyldithio) propionate (Pierce). The reaction was mixed at room temperature overnight, then filtered, washed with DMF, DCM, treated with 50% trifluoroacetic acid in DCM, transferred to a 200 ml Teflon HF reaction vessel and dried in vacuo to give 0.94 gm. 1 ml m-cresol and a magnetic stir bar were added; the vessel was attached to a HF apparatus (Penninsula Labs), cooled to −78° C., evacuated, and 10 ml liquid hydrogen fluoride was condensed in. The reaction was stirred for 1 hour in an ice bath, then the HF was distilled off. The residue was suspended in 150 ml ethyl ether, the resin/peptide mixture was filtered, and washed with ether 2-3 times. The peptide was extracted into aqueous acetic acid which was loaded onto a 2.2×25 cm Vydac C18 reverse phase column. A gradient of 30% to 70% B was run over 450 minutes using Pharmacia FPLC pumps at 4 ml/min (A=0.1% TFA; B=0.1% TFA,50% acetonitrile). Five minute fractions were collected while monitoring the UV absorbance at 214 nm (2.0A). The appropriate fractions (96-100) were combined, frozen and lyophilized, The molecular weight of the material in the combined fractions was determined by LC-mass spectral analysis to be consistent with the correct theoretical molecular weight of 3794.3 for the modified peptide, HVEGTFTSDVSSYLEEQAAKEFIAWLIKGRGK[3-(2-pyridyldithio)propanamide]-amide (SEQ ID NO:22). The purity of the GLP-1 compound from the combined HPLC fractions was approximately 95%.

The GLP-1 compound (SEQ ID NO:22) was conjugated to human serum albumin. 1.3 mg (0.34 μmole) of the GLP-1 compound and 18 mg (0.27 μmole) HSA (Calbiochem) were dissolved in 1 ml phosphate buffered saline (containing 10 mg/ml EDTA), mixed and allowed to set at room temperature. After approximately 18 hours, the reaction mixture was loaded onto a 1.0×25 cm Zorbax C8 reverse phase column and a gradient of 20% to 60% B was run using FPLC pumps at 1 ml/min (A=0.1% TFA; B=0.1% TFA, 90% acetonitrile). Two minute fractions were collected while monitoring the UV absorbance at 214 nm (2.0A). Based on the UV trace, the following fractions were combined in pools, frozen, and lyophilized: A (fractions 53-54, 1.4 mg), B (fractions 55-56, 2.6 mg), C (fractions 57-58, 2.2 mg), D (fractions 59-60), and E (fractions 61-62). MALDI mass spectral analysis of pool C showed a 60:40 ratio HSA to GLP-HSA conjugate (MW=69,700)

The C8 purification products from above pools A-C were re-dissolved in 25 ml water and loaded onto a Pharmacia mono Q HR16/10 column equilibrated in A buffer (0.025 M tris (pH 8.5), 30% acetonitrile). A gradient of 20% to 100% buffer B (buffer B=0.025 M tris (pH 8.5), 30% acetonitrile/0.5M NaCl) was run over 80 minutes at a flow rate of 2 ml/minute while monitoring the UV absorbance at 214 nm (1.0A). Fractions 40-46, comprising the front half of a large peak, were combined, frozen and lyophilized. The dried product was re-dissolved in 2 ml water and was loaded onto a 1.0×25 cm Zorbax C8 column for desalting. A gradient of 20% to 100% B (A=0.1% TFA; B=0.1% TFA, 90% acetonitrile) was run over 80 minutes at a flow rate of 1 ml/minute. Two minute fractions were collected while monitoring the UV absorbance at 214 nm. Fractions 18-19 were combined, frozen and lyophilized to give 1.3 mg of GLP-1 compound-HSA conjugate. MALDI mass spectral analysis showed a significant enhancement of GLP-1 compound-HSA conjugate (90:10) over HSA.

### Example 4

### In Vitro Potency

HEK-293 Aurora CRE-BLAM cells expressing the human GLP-1 receptor are seeded at 20,000 to 40,000 cells/well/100 μl into a 96 well black clear bottom plate. The day after seeding, the medium is replaced with plasma free medium. On the third day after seeding, 20 μl of plasma free medium containing different concentrations of GLP-1 agonist is added to each well to generate a dose response curve. Generally, fourteen dilutions containing from 3 nanomolar to 30 nanomolar GLP-1 compound were used to generate a dose response curve from which EC₅₀ values could be determined. After 5 hours of incubation with GLP-1 compound, 20 μl of β-lactamase substrate (CCF2-AM—Aurora Biosciences—product code 100012) was added and incubation was continued for 1 hour at which point the fluorescence was determined on a cytofluor. The GLP-1 compound-HSA conjugate of Example 3 (HVEGTFTSDVSSYLEEQAAKEFIAWLIKGRGK[3-(2-pyridyldithio)propanamide]-amide (SEQ ID NO:22)) was tested and had EC₅₀ values that were about the same as the activity of Val⁸-GLP-1(7-37)OH.

### Example 5

### Proteolytic Stability

The relative susceptibility of GLP-1 compounds to α-chymotrypsin is assessed in a reaction mixture against the control peptide Val⁸-GLP-1(7-37)OH. A 10 mM phosphate/citrate solution, pH 7.4, is prepared containing a GLP-1 compound at a concentration of 100 μM. A 10 μl aliquot of this solution is then incubated at 4° C. in a 200 ul 10 mM phosphate/citrate solution, pH 7.4, containing 10 mM CaCl₂. Alpha-Chymotrypsin (SIGMA, C-3142 lot 89F8155) is then added to a final concentration of 250 ng/ml. A 10 μl aliquot is removed before addition of the enzyme as well as 20, 40, 60, 80, and 100 minutes following addition of the enzyme. At each time point the aliquot is quenched by adding 90 μl of 20% acetonitrile/0.1% TFA. The proteolytic reaction is followed by injection of 20 μl of the quenched reaction samples onto an analytical Zorbax 300SB-C8 (4.6 mm i.d.×50 mm) column at a 1 ml/min flow rate in 10% acetonitrile/0.075% TFA. Peaks are separated with a gradient of 10 to 90% acetonitrile/0.075% TFA over 15 min. The progress of the enzymatic reaction is followed by plotting loss of peak area of the starting material over time. The rate of proteolytic degradation is calculated from the initial rate of cleavage (timepoint 0 and 20 min) and directly compared to the rate of cleavage of the control peptide Val⁸-GLP-1(7-37)OH. Values above 1 indicate slower rates of initial proteolytic processing as compared to Val⁸-GLP-1(7-37)OH.

1. A GLP-1 compound comprising a GLP-1 peptide modified with a reactive group that reacts with a thiol group on a blood component to form a covalent bond, wherein said reactive group is selected from the group consisting of an activated disulfide bond group or an S-sulfonate.

2. The GLP-1 compound of claim 1, said GLP-1 peptide having the amino acid sequence of formula 1 (SEQ ID NO:1)
Formula 1

| | |
|---|---|
| Xaa₇-Xaa₈-Glu-Gly-Thr-Xaa₁₂-Thr-Ser- | (SEQ ID NO: 1) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Lys-Gly-Arg- | |
| Xaa₃₇ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val, or Ile;
Xaa₃₇ is: L-Cys, D-Cys, homocysteine, or penicillamine;
wherein said GLP-1 peptide is modified at Xaa₃₇; and
provided that the GLP-1 compound does not have the sequence of GLP-1(7-37)OH, GLP-1(7-36)-NH₂, Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser⁸-GLP-1(7-37)OH, Ser⁸-GLP-1(7-36)NH₂, Thr⁸-GLP-1(7-37)OH, Thr⁸-GLP-1(7-36)NH₂, Val⁸-Tyr¹²-GLP-1(7-37)OH, Val⁸-Tyr¹²-GLP-1(7-36)NH₂, Val⁸-Tyr¹⁶-GLP-1(7-37)OH, Val⁸-Tyr¹⁶-GLP-1(7-36)NH₂, Val⁸-Glu²²-GLP-1(7-37)OH, Val⁸-Glu²²-GLP-1(7-36)NH₂, Gly⁸-Glu²²-GLP-1(7-37)OH, Gly⁸-Glu²²-GLP-1(7-36)NH₂, Val⁸-Asp²²-GLP-1(7-37)OH, Val⁸-Asp²²-GLP-1(7-36)NH₂, Gly⁸-Asp²²-GLP-1(7-37)OH, Gly⁸-Asp²²-GLP-1(7-36)NH₂, Val⁸-Lys²²-GLP-1(7-37)OH, Val⁸-Lys²²-GLP-1(7-36)NH₂, Gly⁸-Lys²²-GLP-1(7-37)OH, Gly⁸-Lys²²-GLP-1(7-36)NH₂, Leu⁸-Glu²²-GLP-1(7-37)OH, Leu⁸-Glu²²-GLP-1(7-36)NH₂, Ile⁸-Glu²²-GLP-1(7-37)OH, Ile⁸-Glu²²-GLP-1(7-36)NH₂, Leu⁸-Asp²²-GLP-1(7-37)OH, Leu⁸-Asp²²-GLP-1(7-36)NH₂, Ile⁸-Asp²²-GLP-1(7-37)OH, Ile⁸-Asp²²-GLP-1(7-36)NH₂, Leu⁸-Lys²²-GLP-1(7-37)OH, Leu⁸-Lys²²-GLP-1(7-36)NH₂, Ile⁸-Lys²²-GLP-1(7-37)OH, Ile⁸-Lys²²-GLP-1(7-36)NH₂, Ser⁸-Glu²²-GLP-1(7-37)OH, Ser⁸-Glu²²-GLP-1(7-36)NH₂, Thr⁸-Glu²²-GLP-1(7-37)OH, Thr⁸-Glu²²-GLP-1(7-36)NH₂, Ser⁸-Asp²²-GLP-1(7-37)OH, Ser⁸-Asp²²-GLP-1(7-36)NH₂, Thr⁸-Asp²²-GLP-1(7-37)OH, Thr⁸-Asp²²-GLP-1(7-36)NH₂, Ser⁸-Lys²²-GLP-1(7-37)OH, Ser⁸-Lys²²-GLP-1(7-36)NH₂, Thr⁸-Lys²²-GLP-1(7-37)OH, Thr⁸-Lys²²-GLP-1(7-36)NH₂, Glu²²-GLP-1(7-37)OH, Glu²²-GLP-1(7-36)NH₂, Asp²²-GLP-1(7-37)OH, Asp²²-GLP-1(7-36)NH₂, Lys²²-GLP-1(7-37)OH, Lys²²-GLP-1(7-36)NH₂, Val⁸-Ala²⁷-GLP-1(7-37)OH, Val⁸-Glu²²-Ala²⁷-GLP-1(7-37)OH, Val⁸-Glu³⁰-GLP-1(7-37)OH, Val⁸-Glu³⁰-GLP-1(7-36)NH₂, Gly⁸-Glu³⁰-GLP-1(7-37)OH, Gly⁸-Glu³⁰-GLP-1(7-36)NH₂, Leu⁸-Glu³⁰-GLP-1(7-37)OH, Leu⁸-Glu³⁰-GLP-1(7-36)NH₂, Ile⁸-Glu³⁰-GLP-1(7-37)OH, Ile⁸-Glu³⁰-GLP-1(7-36)NH₂, Ser⁸-Glu³⁰-GLP-1(7-37)OH, Ser⁸-Glu³⁰-GLP-1(7-36)NH₂, Thr⁸-Glu³⁰-GLP-1(7-37)OH, Thr⁸-Glu³⁰-GLP-1(7-36)NH₂, Val⁸-His³⁷-GLP-1(7-37)OH, Val⁸-His³⁷-GLP-1(7-36)NH₂, Gly⁸-His³⁷-GLP-1(7-37)OH, Gly⁸-His³⁷-GLP-1(7-36)NH_{2,} Leu⁸-His³⁷-GLP-1(7-37)OH, Leu⁸-His³⁷-GLP-1(7-36)NH_{2,} Ile⁸-His³⁷-GLP-1(7-37)OH, Ile⁸-His³⁷-GLP-1(7-36)NH_{2,} Ser⁸-His³⁷-GLP-1(7-37)OH, Ser⁸-His³⁷-GLP-1(7-36)NH_{2,} Thr⁸-His³⁷-GLP-1(7-37)OH, Thr⁸-His³⁷-GLP-1(7-36)NH₂.

3. The GLP-1 compound of claim 1, said GLP-1 peptide having the amino acid sequence of formula 2 (SEQ ID NO:2)
Formula 2

| | |
|---|---|
| Xaa₇-Xaa₈-Glu-Gly-Thr-Phe-Thr-Ser- | (SEQ ID NO:2) |
| Asp-Xaa₁₆-Ser-Xaa₁₈-Tyr-Leu-Glu- | |
| Xaa₂₂-Gln-Ala-Xaa₂₅-Lys-Glu-Phe-Ile- | |
| Ala-Trp-Leu-Xaa₃₃-Lys-Gly-Arg-Xaa₃₇ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Gly, Ala, Val, Leu, Ile, Ser, or Thr;
Xaa₁₆ is: Val, Phe, Tyr, or Trp;
Xaa₁₈ is: Ser, Tyr, Trp, Phe, Lys, Ile, Leu, or Val;
Xaa₂₂ is: Gly, Glu, Asp, or Lys;
Xaa₂₅ is: Ala, Val, Ile, or Leu;
Xaa₃₃ is: Val or Ile; and
Xaa₃₇ is: L-Cys, D-Cys, homocysteine, or penicillamine;
wherein said GLP-1 peptide is modified at Xaa₃₇; and
provided that the GLP-1 compound does not have the sequence of GLP-1(7-37)OH, GLP-1(7-36)-NH₂, Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser⁸-GLP-1(7-37)OH, Ser⁸-GLP-1(7-36)NH₂, Thr⁸-GLP-1(7-37)OH, Thr⁸-GLP-1(7-36)NH₂, Val⁸-Tyr¹⁶-GLP-1(7-37)OH, Val⁸-Tyr¹⁶-GLP-1(7-36)NH₂, Val⁸-Glu²²-GLP-1(7-37)OH, Val⁸-Glu²²-GLP-1(7-36)NH₂, Gly⁸-Glu²²-GLP-1(7-37)OH, Gly⁸-Glu²²-GLP-1(7-36)NH₂, Val⁸-Asp²²-GLP-1(7-37)OH, Val⁸-Asp²²-GLP-1(7-36)NH₂, Gly⁸-Asp²²-GLP-1(7-37)OH, Gly⁸-Asp²²-GLP-1(7-36)NH₂, Val⁸-Lys²²-GLP-1(7-37)OH, Val⁸-Lys²²-GLP-1(7-36)NH₂, Gly⁸-Lys²²-GLP-1(7-37)OH, Gly⁸-Lys²²-GLP-1(7-36)NH₂, Leu⁸-Glu²²-GLP-1(7-37)OH, Leu⁸-Glu²²-GLP-1(7-36)NH₂, Ile⁸-Glu²²-GLP-1(7-37)OH, Ile⁸-Glu²²-GLP-1(7-36)NH₂, Leu⁸-Asp²²-GLP-1(7-37)OH, Leu⁸-Asp²²-GLP-1(7-36)NH₂, Ile⁸-Asp²²-GLP-1(7-37)OH, Ile⁸-Asp²²-GLP-1(7-36)NH₂, Leu⁸-Lys²²-GLP-1(7-37)OH, Leu⁸-Lys²²-GLP-1(7-36)NH₂, Ile⁸-Lys²²-GLP-1(7-37)OH, Ile⁸-Lys²² -GLP-1(7-36)NH₂, Ser⁸-Glu²²-GLP-1(7-37)OH, Ser⁸-Glu²²-GLP-1(7-36)NH₂, Thr⁸-Glu²²-GLP-1(7-37)OH, Thr⁸-Glu²²-GLP-1(7-36)NH₂, Ser⁸-Asp²²-GLP-1(7-37)OH, Ser⁸-Asp²²-GLP-1(7-36)NH₂, Thr⁸-Asp²²-GLP-1(7-37)OH, Thr⁸-Asp²²-GLP-1(7-36)NH₂, Ser⁸-Lys²²-GLP-1(7-37)OH, Ser⁸-Lys²²-GLP-1(7-36)NH₂, Thr⁸-Lys²²-GLP-1(7-37)OH, Thr⁸-Lys²²-GLP-1(7-36)NH₂, Glu²²-GLP-1(7-37)OH, Glu²²-GLP-1(7-36)NH₂, Asp²²-GLP-1(7-37)OH, Asp²²-GLP-1(7-36)NH₂, Lys²²-GLP-1(7-37)OH, Lys²²-GLP-1(7-36)NH₂.

4. The GLP-1 compound of claim 1, wherein said GLP-1 peptide is an extended GLP-1 peptide having the amino acid sequence of formula 3 (SEQ ID NO:3)
Formula 3

| | |
|---|---|
| Xaa₇-Xaa₈-Glu-Gly-Thr-Xaa₁₂-Thr-Ser- | (SEQ ID NO:3) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Xaa₃₄-Gly- | |
| Xaa₃₆-Xaa₃₇-Xaa₃₈-Xaa₃₉-Xaa₄₀-Xaa₄₁- | |
| Xaa₄₂-Xaa₄₃-Xaa₄₄-Xaa₄₅-Xaa₄₆-Xaa₄₇- | |
| Xaa₄₈ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val or Ile;
Xaa₃₄ is: Lys, Asp, Arg, or Glu;
Xaa₃₆ is: Gly, Pro, or Arg;
Xaa₃₇ is: Gly, Pro, Ser, L-Cys, D-Cys, homocysteine, or penicillamine;
Xaa₃₈ is: Ser, Pro, His, L-Cys, D-Cys, homocysteine, penicillamine, NH₂;
Xaa₃₉ is: Ser, Arg, Thr, Trp, Lys, L-Cys, D-Cys, homocysteine, penicillamine, NH₂, or is absent;
Xaa₄₀ is: Ser, Gly, L-Cys, D-Cys, homocysteine, penicillamine, NH₂, or is absent;
Xaa₄₁ is: Ala, Asp, Arg, Glu, Lys, Gly, L-Cys, D-Cys, homocysteine, penicillamine, NH₂, or is absent;
Xaa₄₂ is: Pro, Ala, L-Cys, D-Cys, homocysteine, penicillamine, NH₂, or is absent;
Xaa₄₃ is: Pro, Ala, L-Cys, D-Cys, homocysteine, penicillamine, NH₂, or is absent;
Xaa₄₄ is: Pro, Ala, Arg, Lys, His, L-Cys, D-Cys, homocysteine, penicillamine, NH₂, or is absent;
Xaa₄₅ is: Ser, His, Pro, Lys, Arg, L-Cys, D-Cys, homocysteine, penicillamine, NH₂, or is absent;
Xaa₄₆ is: His, Ser, Arg, Lys, L-Cys, D-Cys, homocysteine, penicillamine, NH₂, or is absent;
Xaa₄₇ is: His, Ser, Arg, Lys, L-Cys, D-Cys, homocysteine, penicillamine, NH₂, or is absent; and
Xaa₄₈ is: L-Cys, D-Cys, homocysteine, penicillamine, NH₂, or is absent;
wherein said extended GLP-1 peptide contains a single L-Cys, D-Cys, homocysteine, or penicillamine which occurs at one of Xaa₃₇, Xaa₃₈, Xaa₃₉, Xaa₄₀, Xaa₄₁, Xaa₄₂, Xaa₄₃, Xaa₄₄, Xaa₄₅, Xaa₄₆, Xaa₄₇, or Xaa₄₈, said GLP-1 is modified at said single L-Cys, D-Cys, homocysteine, or penicillamine; and
provided that if Xaa₃₉, Xaa₄₀, Xaa₄₁, Xaa₄₂, Xaa₄₃, Xaa₄₄, Xaa₄₅, Xaa₄₆, or Xaa₄₇ is absent each amino acid downstream is absent and further provided that the GLP-1 peptide does not have the following C-terminal amino acid extension beginning at Xaa₃₆: Gly-Pro-Ser-Ser-Gly-Ala-Pro-Pro-Pro-Ser-NH₂.

5. The GLP-1 compound of claim 1, wherein said GLP-1 peptide is an extended GLP-1 peptide having the amino acid sequence of formula 4 (SEQ ID NO:4)
Formula 4

| | |
|---|---|
| Xaa₇-Xaa₈-Glu-Gly-Thr-Phe-Thr-Ser- | (SEQ ID NO: 4) |
| Asp-Xaa₁₆-Ser-Ser-Tyr-Lys-Glu-Xaa₂₂- | |
| Gln-Ala-Xaa₂₅-Lys-Glu-Phe-Ile-Ala- | |
| Trp-Leu-Xaa₃₃-Xaa₃₄-Gly-Xaa₃₆-Xaa₃₇- | |
| Xaa₃₈-Xaa₃₉-Xaa₄₀-Xaa₄₁-Xaa₄₂-Xaa₄₃- | |
| Xaa₄₄-Xaa₄₅-Xaa₄₆-Xaa₄₇-Xaa₄₈ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Gly, Val, Leu, Ile, Ser, or Thr;
Xaa₁₆ is: Val, Trp, Ile, Leu, Phe, or Tyr;
Xaa₂₂ is: Gly, Glu, Asp, or Lys;
Xaa₂₅ is: Ala, Val, Ile, or Leu;
Xaa₃₃ is: Val or Ile;
Xaa₃₄ is: Lys, Asp, Arg, or Glu;
Xaa₃₆ is: Gly, Pro, or Arg;
Xaa₃₇ is: Gly, Pro, Ser, L-Cys, D-Cys, homocysteine, or penicillamine;
Xaa₃₈ is: Ser, Pro, His, L-Cys, D-Cys, homocysteine, penicillamine, NH₂, or is absent;
Xaa₃₉ is: Ser, Arg, Thr, Trp, Lys, L-Cys, D-Cys, homocysteine, penicillamine, NH₂, or is absent;
Xaa₄₀ is: Ser, Gly, L-Cys, D-Cys, homocysteine, penicillamine, NH₂, or is absent;
Xaa₄₁ is: Ala, Asp, Arg, Glu, Lys, Gly, L-Cys, D-Cys, homocysteine, penicillamine, NH₂, or is absent;
Xaa₄₂ is: Pro, Ala, L-Cys, D-Cys, homocysteine, penicillamine, NH₂, or is absent;
Xaa₄₃ is: Pro, Ala, L-Cys, D-Cys, homocysteine, penicillamine, NH₂, or is absent;
Xaa₄₄ is: Pro, Ala, Arg, Lys, His, L-Cys, D-Cys, homocysteine, penicillamine, NH₂, or is absent;
Xaa₄₅ is: Ser, His, Pro, Lys, Arg, L-Cys, D-Cys, homocysteine, penicillamine, NH₂, or is absent;
Xaa₄₆ is: His, Ser, Arg, Lys, L-Cys, D-Cys, homocysteine, penicillamine, NH₂, or is absent;
Xaa₄₇ is: His, Ser, Arg, Lys, L-Cys, D-Cys, homocysteine, penicillamine, NH₂, or is absent; and
Xaa₄₈ is: L-Cys, D-Cys, homocysteine, penicillamine, NH₂, or is absent;
wherein said extended GLP-1 peptide contains a single L-Cys, D-Cys, homocysteine, or penicillamine which occurs at one of Xaa₃₇, Xaa₃₈, Xaa₃₉, Xaa₄₀, Xaa₄₁, Xaa₄₂, Xaa₄₃, Xaa₄₄, Xaa₄₅, Xaa₄₆, Xaa₄₇, or Xaa₄₈, said GLP-1 is modified at said single L-Cys, D-Cys, homocysteine, or penicillamine; and provided that if Xaa₄₂, Xaa₄₃, Xaa₄₄, Xaa₄₅, Xaa₄₆, or Xaa₄₇ is absent each amino acid downstream is absent and further provided that the GLP-1 peptide does not have the following C-terminal amino acid extension beginning at Xaa₃₆: Gly-Pro-Ser-Ser-Gly-Ala-Pro-Pro-Pro-Ser-NH₂.

6. The GLP-1 compound of claim 1, wherein said GLP-1 peptide is an extended GLP-1 peptide having the amino acid sequence of formula 5 (SEQ ID NO:5)
Formula 5

| | |
|---|---|
| Xaa₇-Xaa₈-Glu-Gly-Thr-Phe-Thr-Ser- | (SEQ ID NO:5) |
| Asp-Val-Ser-Ser-Tyr-Lys-Glu-Xaa₂₂- | |
| Gln-Ala-Xaa₂₅-Lys-Glu-Phe-Ile-Ala- | |
| Trp-Leu-Xaa₃₃-Lys-Gly-Gly-Pro-Xaa₃₈- | |
| Xaa₃₉-Xaa₄₀-Xaa₄₁-Xaa₄₂-Xaa₄₃-Xaa₄₄- | |
| Xaa₄₅-Xaa₄₆-Xaa₄₇-Xaa₄₈ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Gly, Val, Leu, Ile, Ser, or Thr;
Xaa₂₂ is: Gly, Glu, Asp, or Lys;
Xaa₂₅ is: Ala, Val, Ile, or Leu;
Xaa₃₃ is: Val or Ile;
Xaa₃₈ is: Ser, Pro, His, L-Cys, D-Cys, homocysteine, penicillamine, NH₂, or is absent;
Xaa₃₉ is: Ser, Arg, Thr, Trp, Lys, L-Cys, D-Cys, homocysteine, penicillamine, NH₂, or is absent;
Xaa₄₀ is: Ser, Gly, L-Cys, D-Cys, homocysteine, penicillamine, NH₂, or is absent;
Xaa₄₁ is: Ala, Asp, Arg, Glu, Lys, Gly, L-Cys, D-Cys, homocysteine, penicillamine, NH₂, or is absent;
Xaa₄₂ is: Pro, Ala, L-Cys, D-Cys, homocysteine, penicillamine, NH₂, or is absent;
Xaa₄₃ is: Pro, Ala, L-Cys, D-Cys, homocysteine, penicillamine, NH₂, or is absent;
Xaa₄₄ is: Pro, Ala, Arg, Lys, His, L-Cys, D-Cys, homocysteine, penicillamine, NH₂, or is absent;
Xaa₄₅ is: Ser, His, Pro, Lys, Arg, L-Cys, D-Cys, homocysteine, penicillamine, NH₂, or is absent;
Xaa₄₆ is: His, Ser, Arg, Lys, L-Cys, D-Cys, homocysteine, penicillamine, NH₂, or is absent;
Xaa₄₇ is: His, Ser, Arg, Lys, L-Cys, D-Cys, homocysteine, penicillamine, NH₂, or is absent; and
Xaa₄₈ is: L-Cys, D-Cys, homocysteine, penicillamine, NH₂, or is absent;
wherein said extended GLP-1 peptide contains a single L-Cys, D-Cys, homocysteine, or penicillamine which occurs at one of Xaa₃₈, Xaa₃₉, Xaa₄₀, Xaa₄₁, Xaa₄₂, Xaa₄₃, Xaa₄₄, Xaa₄₅, Xaa₄₆, Xaa₄₇, or Xaa₄₈, said GLP-1 is modified at said single L-Cys, D-Cys, homocysteine, or penicillamine; and provided that if Xaa₃₈, Xaa₃₉, Xaa₄₀, Xaa₄₁, Xaa₄₂, Xaa₄₃, Xaa₄₄, Xaa₄₅, Xaa₄₆, or Xaa₄₇ is absent each amino acid downstream is absent.

7. The GLP-1 compound of claim 1, wherein said GLP-1 peptide is an extended GLP-1 peptide having the amino acid sequence of formula 6 (SEQ ID NO:6)
Formula 6

| | |
|---|---|
| Xaa₇-Xaa₈-Glu-Gly-Thr-Xaa₁₂-Thr-Ser- | (SEQ ID NO:6) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Xaa₃₄-Gly- | |
| Xaa₃₆-Xaa₃₇-Xaa₃₈-Xaa₃₉-Xaa₄₀-Xaa₄₁- | |
| Xaa₄₂-Xaa₄₃-Xaa₄₄-Xaa₄₅-Xaa₄₆-Xaa₄₇- | |
| Xaa₄₈-Xaa₄₉-Xaa₅₀-Xaa₅₁ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, or Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val or Ile;
Xaa₃₄ is: Lys, Asp, Arg, or Glu;
Xaa₃₆ is: Gly, Pro, Arg;
Xaa₃₇ is: Gly, Pro, Ser;
Xaa₃₈ is: Ser, Pro, or His;
Xaa₃₉ is: Ser, Arg, Thr, Trp, or Lys;
Xaa₄₀ is: Ser or Gly;
Xaa₄₁ is: Ala, Asp, Arg, Glu, Lys, or Gly;
Xaa₄₂ is: Pro, Ala, L-Cys, D-Cys, homocysteine, penicillamine, NH₂, or is absent;
Xaa₄₃ is: Pro, Ala, L-Cys, D-Cys, homocysteine, penicillamine, NH₂, or is absent;
Xaa₄₄ is: Pro, Ala, Arg, Lys, His, NH₂, L-Cys, D-Cys, homocysteine, penicillamine, NH₂, or is absent;
Xaa₄₅ is: Ser, His, Pro, Lys, Arg, Gly, L-Cys, D-Cys, homocysteine, penicillamine, NH₂, or is absent;
Xaa₄₆ is: His, Ser, Arg, Lys, Pro, Gly, L-Cys, D-Cys, homocysteine, penicillamine, NH₂, or is absent;
Xaa₄₇ is: His, Ser, Arg, Lys, L-Cys, D-Cys, homocysteine, penicillamine, NH₂, or is absent;
Xaa₄₈ is: Gly, His, L-Cys, D-Cys, homocysteine, penicillamine, NH₂, or is absent;
Xaa₄₉ is: Pro, His, L-Cys, D-Cys, homocysteine, penicillamine, NH₂, or is absent;
Xaa₅₀ is: Ser, His, Ser-NH₂, His-NH₂, L-Cys, D-Cys, homocysteine, penicillamine, NH₂, or is absent; and
Xaa₅₁ is: L-Cys, D-Cys, homocysteine, penicillamine, NH₂, or is absent;
wherein said extended GLP-1 peptide contains a single L-Cys, D-Cys, homocysteine, or penicillamine which occurs at one of Xaa₄₂, Xaa₄₃, Xaa₄₄, Xaa₄₅, Xaa₄₆, Xaa₄₇, Xaa₄₉, Xaa_{50,} or Xaa₅₁ said GLP-1 is modified at said single L-Cys, D-Cys, homocysteine, or penicillamine; and provided that if Xaa₄₂, Xaa₄₃, Xaa₄₄, Xaa₄₅, Xaa₄₆, Xaa₄₇, Xaa₄₈, Xaa₄₉, or Xaa₅₀, is absent each amino acid downstream is absent and
further provided that if Xaa₃₆ is Arg and Xaa₃₇ is Gly or Ser, the GLP-1 peptide does not have the following C-terminal amino acid extension beginning at Xaa₃₈: Ser-Ser-Gly-Ala-Pro-Pro-Pro-Ser-NH₂.

8. The GLP-1 compound of claim 1, wherein said GLP-1 peptide is an extended GLP-1 peptide having the amino acid sequence of formula 7 (SEQ ID NO:7)
Formula 7

| | |
|---|---|
| His-Ala-Glu-Gly-Thr-Phe-Thr-Ser-Asp- | (SEQ ID NO:7) |
| Val-Ser-Ser-Tyr-Leu-Glu-Gly-Gln-Ala- | |
| Ala-Lys-Glu-Phe-Ile-Ala-Trp-Leu-Val- | |
| Lys-Gly-Gly-Pro-Xaa₃₈-Xaa₃₉-Xaa₄₀- | |
| Xaa₄₁-Xaa₄₂-Xaa₄₃-Xaa₄₄-Xaa₄₅-Xaa₄₆- | |
| Xaa₄₇-Xaa₄₈-Xaa₄₉-Xaa₅₀-Xaa₅₁ | |

Wherein:
Xaa₃₈ is: Ser, Pro, or His;
Xaa₃₉ is: Ser, Arg, Thr, Trp, or Lys;
Xaa₄₀ is: Ser or Gly;
Xaa₄₁ is: Ala, Asp, Arg, Glu, Lys, or Gly;
Xaa₄₂ is: Pro, Ala, L-Cys, D-Cys, homocysteine, penicillamine, NH₂, or is absent;
Xaa₄₃ is: Pro, Ala, L-Cys, D-Cys, homocysteine, penicillamine, NH₂, or is absent;
Xaa₄₄ is: Pro, Ala, Arg, Lys, His, NH₂, L-Cys, D-Cys, homocysteine, penicillamine, NH₂, or is absent;
Xaa₄₅ is: Ser, His, Pro, Lys, Arg, Gly, L-Cys, D-Cys, homocysteine, penicillamine, NH₂, or is absent;
Xaa₄₆ is: His, Ser, Arg, Lys, Pro, Gly, L-Cys, D-Cys, homocysteine, penicillamine, NH₂, or is absent;
Xaa₄₇ is: His, Ser, Arg, Lys, L-Cys, D-Cys, homocysteine, penicillamine, NH₂, or-is absent;
Xaa₄₈ is: Gly, His, L-Cys, D-Cys, homocysteine, penicillamine, NH₂, or is absent;
Xaa₄₉ is: Pro, His, L-Cys, D-Cys, homocysteine, penicillamine, NH₂, or is absent;
Xaa₅₀ is: Ser, His, Ser-NH₂, His-NH₂, L-Cys, D-Cys, homocysteine, penicillamine, NH₂, or is absent; and
Xaa₅₁ is: L-Cys, D-Cys, homocysteine, penicillamine, NH₂, or is absent;
wherein said extended GLP-1 peptide contains a single L-Cys, D-Cys, homocysteine, or penicillamine which occurs at one of Xaa₄₂, Xaa₄₃, Xaa₄₄, Xaa₄₅, Xaa₄₆, Xaa₄₇, Xaa₄₈, Xaa_{49,} Xaa_{50,} or Xaa₅₁ said GLP-1 is modified at said single L-Cys, D-Cys, homocysteine, or penicillamine; and provided that if Xaa₄₂, Xaa₄₃, Xaa₄₄, Xaa₄₅, Xaa₄₆, Xaa_{47,} Xaa_{48,} Xaa_{49,} or Xaa₅₀, is absent each amino acid downstream is absent.

9. The GLP-1 compound of claim 1, said GLP-1 peptide having the amino acid sequence of formula 8 (SEQ ID NO:8)
Formula 8

| | |
|---|---|
| Xaa₇-Xaa₈-Glu-Gly-Thr-Xaa₁₂-Thr-Ser- | (SEQ ID NO:8) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Lys-Gly-Arg- | |
| Lys | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, Lys;
Xaa₂₅ is: Ala, Val, Ile, or Leu;
Xaa₂₇ is: Glu, Ile, or Ala;
Xaa₃₀ is: Ala or Glu; and
Xaa₃₃ is: Val, or Ile;
wherein said GLP-1 peptide is modified at Lys³⁷; and,
provided that the GLP-1 compound does not have the sequence of GLP-1(7-37)OH, GLP-1(7-36)-NH₂, Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser⁸-GLP-1(7-37)OH, Ser⁸-GLP-1(7-36)NH₂, Thr⁸-GLP-1(7-37)OH, Thr⁸-GLP-1(7-36)NH₂, Val⁸-Tyr¹²-GLP-1(7-37)OH, Val⁸-Tyr¹²-GLP-1(7-36)NH₂, Val⁸-Tyr¹⁶-GLP-1(7-37)OH, Val⁸-Tyr¹⁶-GLP-1(7-36)NH₂, Val⁸-Glu²²-GLP-1(7-37)OH, Val⁸-Glu²²-GLP-1(7-36)NH₂, Gly⁸-Glu²²-GLP-1(7-37)OH, Gly⁸-Glu²²-GLP-1(7-36)NH₂, Val⁸-Asp²²-GLP-1(7-37)OH, Val⁸-Asp²²-GLP-1(7-36)NH₂, Gly⁸-Asp²²-GLP-1(7-37)OH, Gly⁸-Asp²²-GLP-1(7-36)NH₂, Val⁸-Lys²²-GLP-1(7-37)OH, Val⁸-Lys²²-GLP-1(7-36)NH₂, Gly⁸-Lys²²-GLP-1(7-37)OH, Gly⁸-Lys²²-GLP-1(7-36)NH₂, Leu⁸-Glu²²-GLP-1(7-37)OH, Leu⁸-Glu²²-GLP-1(7-36)NH₂, Ile⁸-Glu²²-GLP-1(7-37)OH, Ile⁸-Glu²²-GLP-1(7-36)NH₂, Leu⁸-Asp²²-GLP-1(7-37)OH, Leu⁸-Asp²²-GLP-1(7-36)NH₂, Ile⁸-Asp²²-GLP-1(7-37)OH, Ile⁸-Asp²²-GLP-1(7-36)NH₂, Leu⁸-Lys²²-GLP-1(7-37)OH, Leu⁸-Lys²²-GLP-1(7-36)NH₂, Ile⁸-Lys²²-GLP-1(7-37)OH, Ile⁸-Lys²²-GLP-1(7-36)NH₂, Ser⁸-Glu²²-GLP-1(7-37)OH, Ser⁸-Glu²²-GLP-1(7-36)NH₂, Thr⁸-Glu²²-GLP-1(7-37)OH, Thr⁸-Glu²²-GLP-1(7-36)NH₂, Ser⁸-Asp²²-GLP-1(7-37)OH, Ser⁸-Asp²²-GLP-1(7-36)NH₂, Thr⁸-Asp²²-GLP-1(7-37)OH, Thr⁸-Asp²²-GLP-1(7-36)NH₂, Ser⁸-Lys²²-GLP-1(7-37)OH, Ser⁸-Lys²²-GLP-1(7-36)NH₂, Thr⁸-Lys²²-GLP-1(7-37)OH, Thr⁸-Lys²²-GLP-1(7-36)NH₂, Glu²²-GLP-1(7-37)OH, Glu²²-GLP-1(7-36)NH₂, Asp²²-GLP-1(7-37)OH, Asp²²-GLP-1(7-36)NH₂, Lys²²-GLP-1(7-37)OH, Lys²²-GLP-1(7-36)NH₂, Val⁸-Ala²⁷-GLP-1(7-37)OH, Val⁸-Glu²²-Ala²⁷-GLP-1(7-37)OH, Val⁸-Glu³⁰-GLP-1(7-37)OH, Val⁸-Glu³⁰-GLP-1(7-36)NH₂, Gly⁸-Glu³⁰-GLP-1(7-37)OH, Gly⁸-Glu³⁰-GLP-1(7-36)NH₂, Leu⁸-Glu³⁰-GLP-1(7-37)OH, Leu⁸-Glu³⁰-GLP-1(7-36)NH₂, Ile⁸-Glu³⁰-GLP-1(7-37)OH, Ile⁸-Glu³⁰-GLP-1(7-36)NH₂, Ser⁸-Glu³⁰-GLP-1(7-37)OH, Ser⁸-Glu³⁰-GLP-1(7-36)NH₂, Thr⁸-Glu³⁰-GLP-1(7-37)OH, Thr⁸-Glu³⁰-GLP-1(7-36)NH₂, Val⁸-His³⁷-GLP-1(7-37)OH, Val⁸-His³⁷-GLP-1(7-36)NH₂, Gly⁸-His³⁷-GLP-1(7-37)OH, Gly⁸-His³⁷-GLP-1(7-36)NH_{2,} Leu⁸-His³⁷-GLP-1(7-37)OH, Leu⁸-His³⁷-GLP-1(7-36)NH_{2,} Ile⁸-His³⁷-GLP-1(7-37)OH, Ile⁸-His³⁷-GLP-1(7-36)NH_{2,} Ser⁸-His³⁷-GLP-1(7-37)OH, Ser⁸-His³⁷-GLP-1(7-36)NH_{2,} Thr⁸-His³⁷-GLP-1(7-37)OH, Thr⁸-His³⁷-GLP-1(7-36)NH₂, Lys³⁷-GLP-1(7-37)OH.

10. The GLP-1 compound of claim 1, said GLP-1 peptide having the amino acid sequence of formula 9 (SEQ ID NO:9)
Formula 9

| | |
|---|---|
| Xaa₇-Xaa₈-Glu-Gly-Thr-Phe-Thr-Ser- | (SEQ ID NO:9) |
| Asp-Xaa₁₆-Ser-Xaa₁₈-Tyr-Leu-Glu- | |
| Xaa₂₂-Gln-Ala-Xaa₂₅-Lys-Glu-Phe-Ile- | |
| Ala-Trp-Leu-Xaa₃₃-Lys-Gly-Arg-Lys | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Gly, Ala, Val, Leu, Ile, Ser, or Thr;
Xaa₁₆ is: Val, Phe, Tyr, or Trp;
Xaa₁₈ is: Ser, Tyr, Trp, Phe, Lys, Ile, Leu, or Val;
Xaa₂₂ is: Gly, Glu, Asp, or Lys;
Xaa₂₅ is: Ala, Val, Ile, or Leu; and
Xaa₃₃ is: Val or Ile;
wherein said GLP-1 peptide is modified at Lys³⁷; and,
provided that the GLP-1 compound does not have the sequence of GLP-1(7-37)OH, GLP-1(7-36)-NH₂, Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸ -GLP-1(7-36)NH₂, Ser⁸ -GLP-1(7-37)OH, Ser⁸-GLP-1(7-36)NH₂, Thr⁸-GLP-1(7-37)OH, Thr⁸-GLP-1(7-36)NH₂, Val⁸-Tyr¹²-GLP-1(7-37)OH, Val⁸-Tyr¹²-GLP-1(7-36)NH₂, Val⁸-Tyr¹⁶-GLP-1(7-37)OH, Val⁸-Tyr¹⁶-GLP-1(7-36)NH₂, Val⁸-Glu²²-GLP-1(7-37)OH, Val⁸-Glu²²-GLP-1(7-36)NH₂, Gly⁸-Glu²²-GLP-1(7-37)OH, Gly⁸-Glu²²-GLP-1(7-36)NH₂, Val⁸-Asp²²-GLP-1(7-37)OH, Val⁸-Asp²²-GLP-1(7-36)NH₂, Gly⁸-Asp²²-GLP-1(7-37)OH, Gly⁸-Asp²²-GLP-1(7-36)NH₂, Val⁸-Lys²²-GLP-1(7-37)OH, Val⁸-Lys²²-GLP-1(7-36)NH₂, Gly⁸-Lys²²-GLP-1(7-37)OH, Gly⁸-Lys²²-GLP-1(7-36)NH₂, Leu⁸-Glu²²-GLP-1(7-37)OH, Leu⁸-Glu²²-GLP-1(7-36)NH₂, Ile⁸-Glu²²-GLP-1(7-37)OH, Ile⁸-Glu²²-GLP-1(7-36)NH₂, Leu⁸-Asp²²-GLP-1(7-37)OH, Leu⁸-Asp²²-GLP-1(7-36)NH₂, Ile⁸-Asp²²-GLP-1(7-37)OH, Ile⁸-Asp²²-GLP-1(7-36)NH₂, Leu⁸-Lys²²-GLP-1(7-37)OH, Leu⁸-Lys²²-GLP-1(7-36)NH₂, Ile⁸-Lys²²-GLP-1(7-37)OH, Ile⁸-Lys²²-GLP-1(7-36)NH₂, Ser⁸-Glu²²-GLP-1(7-37)OH, Ser⁸-Glu²²-GLP-1(7-36)NH₂, Thr⁸-Glu²²-GLP-1(7-37)OH, Thr⁸-Glu²²-GLP-1(7-36)NH₂, Ser⁸-Asp²²-GLP-1(7-37)OH, Ser⁸-Asp²²-GLP-1(7-36)NH₂, Thr⁸-Asp²²-GLP-1(7-37)OH, Thr⁸-Asp²²-GLP-1(7-36)NH₂, Ser⁸-Lys²²-GLP-1(7-37)OH, Ser⁸-Lys²²-GLP-1(7-36)NH₂, Thr⁸-Lys²²-GLP-1(7-37)OH, Thr⁸-Lys²²-GLP-1(7-36)NH₂, Glu²²-GLP-1(7-37)OH, Glu²²-GLP-1(7-36)NH₂, Asp²²-GLP-1(7-37)OH, Asp²²-GLP-1(7-36)NH₂, Lys²²-GLP-1(7-37)OH, Lys²²-GLP-1(7-36)NH₂, Val⁸-Ala²⁷-GLP-1(7-37)OH, Val⁸-Glu²²-Ala²⁷-GLP-1(7-37)OH, Val⁸-Glu³⁰-GLP-1(7-37)OH, Val⁸-Glu³⁰-GLP-1(7-36)NH₂, Gly⁸-Glu³⁰-GLP-1(7-37)OH, Gly⁸-Glu³⁰-GLP-1(7-36)NH₂, Leu⁸-Glu³⁰-GLP-1(7-37)OH, Leu⁸-Glu³⁰-GLP-1(7-36)NH₂, Ile⁸-Glu³⁰-GLP-1(7-37)OH, Ile⁸-Glu³⁰-GLP-1(7-36)NH₂, Ser⁸-Glu³⁰-GLP-1(7-37)OH, Ser⁸-Glu³⁰-GLP-1(7-36)NH₂, Thr⁸-Glu³⁰-GLP-1(7-37)OH, Thr⁸-Glu³⁰-GLP-1(7-36)NH₂, Val⁸-His³⁷-GLP-1(7-37)OH, Val⁸-His³⁷-GLP-1(7-36)NH₂, Gly⁸-His³⁷-GLP-1(7-37)OH, Gly⁸-His³⁷-GLP-1(7-36)NH_{2,} Leu⁸-His³⁷-GLP-1(7-37)OH, Leu⁸-His³⁷-GLP-1(7-36)NH_{2,} Ile⁸-His³⁷-GLP-1(7-37)OH, Ile⁸-His³⁷-GLP-1(7-36)NH_{2,} Ser⁸-His³⁷-GLP-1(7-37)OH, Ser⁸-His³⁷-GLP-1(7-36)NH_{2,} Thr⁸-His³⁷-GLP-1(7-37)OH, Thr⁸-His³⁷-GLP-1(7-36)NH₂, Lys³⁷-GLP-1(7-37)OH.

11. The GLP-1 compound of claim 1, wherein said GLP-1 peptide is an extended GLP-1 peptide having the amino acid sequence of formula 10 (SEQ ID NO:10)
Formula 10

| | |
|---|---|
| Xaa₇-Xaa₈-Glu-Gly-Thr-Xaa₁₂-Thr-Ser- | (SEQ ID NO:10) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Xaa₃₄-Gly- | |
| Xaa₃₆-Xaa₃₇-Xaa₃₈-Xaa₃₉-Xaa₄₀-Xaa₄₁- | |
| Xaa₄₂-Xaa₄₃-Xaa₄₄-Xaa₄₅-Xaa₄₆-Xaa₄₇- | |
| Xaa₄₈ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, or Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val or Ile;
Xaa₃₄ is: Lys, Asp, Arg, or Glu;
Xaa₃₆ is: Gly, Pro, or Arg;
Xaa₃₇ is: Gly, Pro, Ser, or Lys;
Xaa₃₈ is: Ser, Pro, His, Lys, NH₂;
Xaa₃₉ is: Ser, Arg, Thr, Trp, Lys, NH₂, or is absent;
Xaa₄₀ is: Ser, Gly, Lys, NH₂, or is absent;
Xaa₄₁ is: Ala, Asp, Arg, Glu, Lys, Gly, NH₂, or is absent;
Xaa₄₂ is: Pro, Ala, Lys, NH₂, or is absent;
Xaa₄₃ is: Pro, Ala, Lys, NH₂, or is absent;
Xaa₄₄ is: Pro, Ala, Arg, Lys, His, NH₂, or is absent;
Xaa₄₅ is: Ser, His, Pro, Lys, Arg, NH₂, or is absent;
Xaa₄₆ is: His, Ser, Arg, Lys, NH₂, or is absent;
Xaa₄₇ is: His, Ser, Arg, Lys, NH₂, or is absent; and
Xaa₄₈ is: Lys, NH₂, or is absent;
wherein said extended GLP-1 peptide is modified at a single Lys which occurs at one of Xaa₃₇, Xaa₃₈, Xaa₃₉, Xaa₄₀, Xaa₄₁, Xaa₄₂, Xaa₄₃, Xaa₄₄, Xaa₄₅, Xaa₄₆, Xaa₄₇, or Xaa₄₈; and provided that if Xaa₃₉, Xaa₄₀, Xaa₄₁, Xaa₄₂, Xaa₄₃, Xaa₄₄, Xaa₄₅, Xaa₄₆, or Xaa₄₇ is absent each amino acid downstream is absent and further provided that the GLP-1 peptide does not have the following C-terminal amino acid extension beginning at Xaa₃₆: Gly-Pro-Ser-Ser-Gly-Ala-Pro-Pro-Pro-Ser-NH₂.

12. The GLP-1 compound of claim 1, wherein said GLP-1 peptide is an extended GLP-1 peptide having the amino acid sequence of formula 11 (SEQ ID NO:11)
Formula 11

| | |
|---|---|
| Xaa₇-Xaa₈-Glu-Gly-Thr-Phe-Thr-Ser- | (SEQ ID NO:11) |
| Asp-Xaa₁₆-Ser-Ser-Tyr-Lys-Glu-Xaa₂₂- | |
| Gln-Ala-Xaa₂₅-Lys-Glu-Phe-Ile-Ala- | |
| Trp-Leu-Xaa₃₃-Xaa₃₄-Gly-Xaa₃₆-Xaa₃₇- | |
| Xaa₃₈-Xaa₃₉-Xaa₄₀-Xaa₄₁-Xaa₄₂-Xaa₄₃- | |
| Xaa₄₄-Xaa₄₅-Xaa₄₆-Xaa₄₇-Xaa₄₈ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₁₆ is: Val, Trp, Ile, Leu, Phe, or Tyr;
Xaa₂₂ is: Gly, Glu, Asp, or Lys;
Xaa₂₅ is: Ala, Val, Ile, or Leu;
Xaa₃₃ is: Val or Ile;
Xaa₃₄ is: Lys, Asp, Arg, or Glu;
Xaa₃₆ is: Gly, Pro, or Arg;
Xaa₃₇ is: Gly, Pro, Ser, Lys;
Xaa₃₈ is: Ser, Pro, His, Lys, NH₂, or is absent;
Xaa₃₉ is: Ser, Arg, Thr, Trp, Lys, NH₂, or is absent;
Xaa₄₀ is: Ser, Gly, Lys, NH₂, or is absent;
Xaa₄₁ is: Ala, Asp, Arg, Glu, Lys, Gly, NH₂, or is absent;
Xaa₄₂ is: Pro, Ala, Lys, NH₂, or is absent;
Xaa₄₃ is: Pro, Ala, Lys, NH₂, or is absent;
Xaa₄₄ is: Pro, Ala, Arg, Lys, His, NH₂, or is absent;
Xaa₄₅ is: Ser, His, Pro, Lys, Arg, NH₂, or is absent;
Xaa₄₆ is: His, Ser, Arg, Lys, NH₂, or is absent;
Xaa₄₇ is: His, Ser, Arg, Lys, NH₂, or is absent; and
Xaa₄₈ is: Lys, NH₂, or is absent;
wherein said extended GLP-1 peptide is modified at a single Lys which occurs at one of Xaa₃₇, Xaa₃₈, Xaa₃₉, Xaa₄₀, Xaa₄₁, Xaa₄₂, Xaa₄₃, Xaa₄₄, Xaa₄₅, Xaa₄₆, Xaa₄₇, or Xaa₄₈; and provided that if Xaa₃₉, Xaa₄₀, Xaa₄₁, Xaa₄₂, Xaa₄₃, Xaa₄₄, Xaa₄₅, Xaa₄₆, or Xaa₄₇ is absent each amino acid downstream is absent and further provided that the GLP-1 peptide does not have the following C-terminal amino acid extension beginning at Xaa₃₆: Gly-Pro-Ser-Ser-Gly-Ala-Pro-Pro-Pro-Ser-NH₂.

13. The GLP-1 compound of claim 1, wherein said GLP-1 peptide is an extended GLP-1 peptide having the amino acid sequence of formula 12 (SEQ ID NO:12)
Formula 12

| | |
|---|---|
| Xaa₇-Xaa₈-Glu-Gly-Thr-Phe-Thr-Ser- | (SEQ ID NO:12) |
| Asp-Val-Ser-Ser-Tyr-Lys-Glu-Xaa₂₂- | |
| Gln-Ala-Xaa₂₅-Lys-Glu-Phe-Ile-Ala- | |
| Trp-Leu-Xaa₃₃-Lys-Gly-Gly-Pro-Xaa₃₈- | |
| Xaa₃₉-Xaa₄₀-Xaa₄₁-Xaa₄₂-Xaa₄₃-Xaa₄₄- | |
| Xaa₄₅-Xaa₄₆-Xaa₄₇-Xaa₄₈ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Gly, Val, Leu, Ile, Ser, or Thr;
Xaa₂₂ is: Gly, Glu, Asp, Lys;
Xaa₂₅ is: Ala, Val, Ile, or Leu;
Xaa₃₃ is: Val or Ile;
Xaa₃₈ is: Ser, Pro, His, Lys, NH₂, or is absent;
Xaa₃₉ is: Ser, Arg, Thr, Trp, Lys, NH₂, or is absent;
Xaa₄₀ is: Ser, Gly, Lys, NH₂, or is absent;
Xaa₄₁ is: Ala, Asp, Arg, Glu, Lys, Gly, NH₂, or is absent;
Xaa₄₂ is: Pro, Ala, Lys, NH₂, or is absent;
Xaa₄₃ is: Pro, Ala, Lys, NH₂, or is absent;
Xaa₄₄ is: Pro, Ala, Arg, Lys, His, NH₂, or is absent;
Xaa₄₅ is: Ser, His, Pro, Lys, Arg, NH₂, or is absent;
Xaa₄₆ is: His, Ser, Arg, Lys, NH₂, or is absent;
Xaa₄₇ is: His, Ser, Arg, Lys, NH₂, or is absent; and
Xaa₄₈ is: Lys, NH₂, or is absent;
wherein said extended GLP-1 peptide is modified at a single Lys which occurs at one of Xaa₃₇, Xaa₃₈, Xaa₃₉, Xaa₄₀, Xaa₄₁, Xaa₄₂, Xaa₄₃, Xaa₄₄, Xaa₄₅, Xaa₄₆, Xaa₄₇, or Xaa₄₈; and provided that if Xaa₃₈, Xaa₃₉, Xaa₄₀, Xaa₄₁, Xaa₄₂, Xaa₄₃, Xaa₄₄, Xaa₄₅, Xaa₄₆, or Xaa₄₇ is absent each amino acid downstream is absent.

14. The GLP-1 compound of claim 1, wherein said GLP-1 peptide is an extended GLP-1 peptide having the amino acid sequence of formula 13 (SEQ ID NO:13)
Formula 13

| | |
|---|---|
| Xaa₇-Xaa₈-Glu-Gly-Thr-Xaa₁₂-Thr-Ser- | (SEQ ID NO:13) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Xaa₃₄-Gly- | |
| Xaa₃₆-Xaa₃₇-Xaa₃₈-Xaa₃₉-Xaa₄₀-Xaa₄₁- | |
| Xaa₄₂-Xaa₄₃-Xaa₄₄-Xaa₄₅-Xaa₄₆-Xaa₄₇- | |
| Xaa₄₈-Xaa₄₉-Xaa₅₀-Xaa₅₁ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, or Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val or Ile;
Xaa₃₄ is: Lys, Asp, Arg, or Glu;
Xaa₃₆ is: Gly, Pro, or Arg;
Xaa₃₇ is: Gly, Pro, Ser;
Xaa₃₈ is: Ser, Pro, or His;
Xaa₃₉ is: Ser, Arg, Thr, Trp, or Lys;
Xaa₄₀ is: Ser or Gly;
Xaa₄₁ is: Ala, Asp, Arg, Glu, Lys, or Gly;
Xaa₄₂ is: Pro, Ala, Lys, NH₂, or is absent;
Xaa₄₃ is: Pro, Ala, Lys, NH₂, or is absent;
Xaa₄₄ is: Pro, Ala, Arg, Lys, His, NH₂, or is absent;
Xaa₄₅ is: Ser, His, Pro, Lys, Arg, NH₂, or is absent;
Xaa₄₆ is: His, Ser, Arg, Lys, NH₂, or is absent;
Xaa₄₇ is: His, Ser, Arg, Lys, NH₂, or is absent; and
Xaa₄₈ is: Lys, NH₂, or is absent;
Xaa₄₉ is: Pro, His, Lys, NH₂, or is absent;
Xaa₅₀ is: Ser, His, Lys, NH₂, or is absent; and
Xaa₅₁ is: Lys, NH₂, or is absent;
wherein said extended GLP-1 peptide is modified at a single Lys which occurs at one of Xaa₄₂, Xaa₄₃, Xaa₄₄, Xaa₄₅, Xaa₄₆, Xaa₄₇, Xaa₄₈, Xaa_{49,} Xaa_{50,} or Xaa₅₁; and
provided that if Xaa₃₈, Xaa₃₉, Xaa₄₀, Xaa₄₁, Xaa₄₂, Xaa₄₃, Xaa₄₄, Xaa₄₅, Xaa₄₆, Xaa_{47,} Xaa_{48,} Xaa_{49,} or Xaa₅₀, is absent each amino acid downstream is absent.

15. The GLP-1 compound of claim 1, wherein said GLP-1 peptide is an extended GLP-1 peptide having the amino acid sequence of formula 14 (SEQ ID NO:14)
Formula 14

| | |
|---|---|
| His-Ala-Glu-Gly-Thr-Phe-Thr-Ser-Asp- | (SEQ ID NO:14) |
| Val-Ser-Ser-Tyr-Leu-Glu-Gly-Gln-Ala- | |
| Ala-Lys-Glu-Phe-Ile-Ala-Trp-Leu-Val- | |
| Lys-Gly-Gly-Pro-Xaa₃₈-Xaa₃₉-Xaa₄₀- | |
| Xaa₄₁-Xaa₄₂-Xaa₄₃-Xaa₄₄-Xaa₄₅-Xaa₄₆- | |
| Xaa₄₇-Xaa₄₈-Xaa₄₉-Xaa₅₀-Xaa₅₁ | |

Wherein:
Xaa₃₈ is: Ser, Pro, His;
Xaa₃₉ is: Ser, Arg, Thr, Trp, or Lys;
Xaa₄₀ is: Ser or Gly;
Xaa₄₁ is: Ala, Asp, Arg, Glu, Lys, or Gly;
Xaa₄₂ is: Pro, Ala, Lys, NH₂, or is absent;
Xaa₄₃ is: Pro, Ala, Lys, NH₂, or is absent;
Xaa₄₄ is: Pro, Ala, Arg, Lys, His, NH₂, or is absent;
Xaa₄₅ is: Ser, His, Pro, Lys, Arg, His, Nh₂, or is absent;
Xaa₄₆ is: His, Ser, Arg, Lys, NH₂, or is absent;
Xaa₄₇ is: His, Ser, Arg, Lys, NH₂, or is absent; and
Xaa₄₈ is: Lys, NH₂, or is absent;
Xaa₄₉ is: Pro, His, Lys, NH₂, or is absent;
Xaa₅₀ is: Ser, His, Lys, NH₂, or is absent; and
Xaa₅₁ is: Lys, NH₂, or is absent;
wherein said extended GLP-1 peptide is modified at a single Lys which occurs at one of Xaa₄₂, Xaa₄₃, Xaa₄₄, Xaa₄₅, Xaa₄₆, Xaa₄₇, Xaa₄₈, Xaa_{49,} Xaa_{50,} or Xaa₅₁; and
provided that if Xaa₄₂, Xaa₄₃, Xaa₄₄, Xaa₄₅, Xaa₄₆, Xaa_{47,} Xaa_{48,} Xaa_{49,} or Xaa₅₀, is absent each amino acid downstream is absent.

16. The GLP-1 compound of any of claim 1 wherein said reactive group is an activated disulfide bond group.

17. The GLP-1 compound of claim 1 wherein said reactive group is an S-sulfonate.

18. A GLP-1 compound comprising a GLP-1 peptide modified with a reactive group that reacts with an amino group, a hydroxyl group, or a thiol group on a blood component to form a covalent bond, wherein said reactive group is selected from the group consisting of a succinimidyl group and a maleimido group, said GLP-1 peptide having the amino acid sequence of formula 15 (SEQ ID NO:15)
Formula 15

| | |
|---|---|
| Xaa₇-Xaa₈-Glu-Gly-Thr-Xaa₁₂-Thr-Ser- | (SEQ ID NO:15) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Lys-Gly-Arg- | |
| Xaa₃₇ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val, or Ile; and
Xaa₃₇ is: Gly, His, Lys, or NH₂, or is absent,
provided that the GLP-1 compound does not have the sequence of GLP-1(7-37)OH, GLP-1(7-36)-NH₂, Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser⁸-GLP-1(7-37)OH, Ser⁸-GLP-1(7-36)NH₂, Thr⁸-GLP-1(7-37)OH, Thr⁸-GLP-1(7-36)NH₂, Val⁸-Tyr¹²-GLP-1(7-37)OH, Val⁸-Tyr¹²-GLP-1(7-36)NH₂, Val⁸-Tyr¹⁶-GLP-1(7-37)OH, Val⁸-Tyr¹⁶-GLP-1(7-36)NH₂, Val⁸-Glu²²-GLP-1(7-37)OH, Val⁸-Glu²²-GLP-1(7-36)NH₂, Gly⁸-Glu²²-GLP-1(7-37)OH, Gly⁸-Glu²²-GLP-1(7-36)NH₂, Val⁸-Asp²²-GLP-1(7-37)OH, Val⁸-Asp²²-GLP-1(7-36)NH₂, Gly⁸-Asp²²-GLP-1(7-37)OH, Gly⁸-Asp²²-GLP-1(7-36)NH₂, Val⁸-Lys²²-GLP-1(7-37)OH, Val⁸-Lys²²-GLP-1(7-36)NH₂, Gly⁸-Lys²²-GLP-1(7-37)OH, Gly⁸-Lys²²-GLP-1(7-36)NH₂, Leu⁸-Glu²²-GLP-1(7-37)OH, Leu⁸-Glu²²-GLP-1(7-36)NH₂, Ile⁸-Glu²²-GLP-1(7-37)OH, Ile⁸-Glu²²-GLP-1(7-36)NH₂, Leu⁸-Asp²²-GLP-1(7-37)OH, Leu⁸-Asp²²-GLP-1(7-36)NH₂, Ile⁸-Asp²²-GLP-1(7-37)OH, Ile⁸-Asp²²-GLP-1(7-36)NH₂, Leu⁸-Lys²²-GLP-1(7-37)OH, Leu⁸-Lys²²-GLP-1(7-36)NH₂, Ile⁸-Lys²²-GLP-1(7-37)OH, Ile⁸-Lys²²-GLP-1(7-36)NH₂, Ser⁸-Glu²²-GLP-1(7-37)OH, Ser⁸-Glu²²-GLP-1(7-36)NH₂, Thr⁸-Glu²²-GLP-1(7-37)OH, Thr⁸-Glu²²-GLP-1(7-36)NH₂, Ser⁸-Asp²²-GLP-1(7-37)OH, Ser⁸-Asp²²-GLP-1(7-36)NH₂, Thr⁸-Asp²²-GLP-1(7-37)OH, Thr⁸-Asp²²-GLP-1(7-36)NH₂, Ser⁸-Lys²²-GLP-1(7-37)OH, Ser⁸-Lys²²-GLP-1(7-36)NH₂, Thr⁸-Lys²²-GLP-1(7-37)OH, Thr⁸-Lys²²-GLP-1(7-36)NH₂, Glu²²-GLP-1(7-37)OH, Glu²²-GLP-1(7-36)NH₂, Asp²²-GLP-1(7-37)OH, Asp²²-GLP-1(7-36)NH₂, Lys²²-GLP-1(7-37)OH, Lys²²-GLP-1(7-36)NH₂, Val⁸-Ala²⁷-GLP-1(7-37)OH, Val⁸-Glu²²-Ala²⁷-GLP-1(7-37)OH, Val⁸-Glu³⁰-GLP-1(7-37)OH, Val⁸-Glu³⁰-GLP-1(7-36)NH₂, Gly⁸-Glu³⁰-GLP-1(7-37)OH, Gly⁸-Glu³⁰-GLP-1(7-36)NH₂, Leu⁸-Glu³⁰-GLP-1(7-37)OH, Leu⁸-Glu³⁰-GLP-1(7-36)NH₂, Ile⁸-Glu³⁰-GLP-1(7-37)OH, Ile⁸-Glu³⁰-GLP-1(7-36)NH₂, Ser⁸-Glu³⁰-GLP-1(7-37)OH, Ser⁸-Glu³⁰-GLP-1(7-36)NH₂, Thr⁸-Glu³⁰-GLP-1(7-37)OH, Thr⁸-Glu³⁰-GLP-1(7-36)NH₂, Val⁸-His³⁷-GLP-1(7-37)OH, Val⁸-His³⁷-GLP-1(7-36)NH_{2,} Gly⁸-His³⁷-GLP-1(7-37)OH, Gly⁸-His³⁷-GLP-1(7-36)NH_{2,} Leu⁸-His³⁷-GLP-1(7-37)OH, Leu⁸-His³⁷-GLP-1(7-36)NH_{2,} Ile⁸-His³⁷-GLP-1(7-37)OH, Ile⁸-His³⁷-GLP-1(7-36)NH_{2,} Ser⁸-His³⁷-GLP-1(7-37)OH, Ser⁸-His³⁷-GLP-1(7-36)NH_{2,} Thr⁸-His³⁷-GLP-1(7-37)OH, Thr⁸-His³⁷-GLP-1(7-36)NH₂, Lys³⁷-GLP-1(7-37)OH.

19. The GLP-1 compound of claim 18, wherein Xaa₃₇ of said GLP-1 peptide is Lys and said GLP-1 peptide is modified at Xaa₃₇.

20. A GLP-1 compound comprising an extended GLP-1 peptide modified with a reactive group that reacts with an amino group, a hydroxyl group, or a thiol group on a blood component to form a covalent bond, wherein said reactive group is selected from the group consisting of a succinimidyl group and a maleimido group, said extended GLP-1 peptide having the amino acid sequence of formula 10 (SEQ ID NO:10)
Formula 10

| | |
|---|---|
| Xaa₇-Xaa₈-Glu-Gly-Thr-Xaa₁₂-Thr-Ser- | (SEQ ID NO:10) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Xaa₃₄-Gly- | |
| Xaa₃₆-Xaa₃₇-Xaa₃₈-Xaa₃₉-Xaa₄₀-Xaa₄₁- | |
| Xaa₄₂-Xaa₄₃-Xaa₄₄-Xaa₄₅-Xaa₄₆-Xaa₄₇- | |
| Xaa₄₈ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, or Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val or Ile;
Xaa₃₄ is: Lys, Asp, Arg, or Glu;
Xaa₃₆ is: Gly, Pro, or Arg;
Xaa₃₇ is: Ser, Pro, His, or Lys;
Xaa₃₈ is: Ser, Pro, His, or Lys;
Xaa₃₉ is: Ser, Arg, Thr, Trp, Lys, NH₂, or is absent;
Xaa₄₀ is: Ser, Gly, Lys, NH₂, or is absent;
Xaa₄₁ is: Ala, Asp, Arg, Glu, Lys, Gly, Lys, NH₂, or is absent;
Xaa₄₃ is: Pro, Ala, Lys, NH₂, or is absent;
Xaa₄₄ is: Pro, Ala, Arg, Lys, His, NH₂, or is absent;
Xaa₄₅ is: Ser, His, Pro, Lys, Arg, NH₂ or is absent;
Xaa₄₆ is: His, Ser, Arg, Lys, NH₂ or is absent;
Xaa₄₇ is: His, Ser, Arg, Lys, NH₂ or is absent; and
Xaa₄₈ is Lys, NH₂, or is absent;
provided that if Xaa_{39,} Xaa₄₀, Xaa_{41,} Xaa₄₂, Xaa₄₃, Xaa₄₄, Xaa₄₅, Xaa₄₆, or Xaa₄₇ is absent each amino acid downstream is absent and further provided that the GLP-1 peptide does not have the following C-terminal amino acid extension beginning at Xaa₃₆: Gly-Pro-Ser-Ser-Gly-Ala-Pro-Pro-Pro-Ser-NH₂.

21. The GLP-1 compound of claim 20, wherein said GLP-1 peptide is modified at a Lys, and said Lys occurs at either Xaa₃₇, Xaa₃₈, Xaa₃₉, Xaa₄₀, Xaa₄₁, Xaa₄₂, Xaa₄₃, Xaa₄₄, Xaa₄₅, Xaa₄₆, Xaa₄₇, or Xaa₄₈.

22. A GLP-1 compound comprising an extended GLP-1 peptide modified with a reactive group that reacts with an amino group, a hydroxyl group, or a thiol group on a blood component to form a covalent bond, wherein said reactive group is selected from the group consisting of a succinimidyl group and a maleimido group, said extended GLP-1 peptide having the amino acid sequence of formula 13 (SEQ ID NO:13)
Formula 13

| | |
|---|---|
| Xaa₇-Xaa₈-Glu-Gly-Thr-Xaa₁₂-Thr-Ser- | (SEQ ID NO:13) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Xaa₃₄-Gly- | |
| Xaa₃₆-Xaa₃₇-Xaa₃₈-Xaa₃₉-Xaa₄₀-Xaa₄₁- | |
| Xaa₄₂-Xaa₄₃-Xaa₄₄-Xaa₄₅-Xaa₄₆-Xaa₄₇- | |
| Xaa₄₈-Xaa₄₉-Xaa₅₀-Xaa₅₁ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, or Lys;
Xaa₂₅ is: Ala, Val, Ile, or Leu;
Xaa₂₇ is: Glu, Ile, or, Ala;
Xaa₃₀ is: Ala or, Glu
Xaa₃₃ is: Val or, Ile;
Xaa₃₄ is: Lys, Asp, Arg, or Glu;
Xaa₃₆ is: Gly, Pro, or Arg;
Xaa₃₇ is: Gly, Pro, or Ser;
Xaa₃₈ is: Ser, Pro, or His;
Xaa₃₉ is: Ser, Arg, Thr, Trp, or Lys;
Xaa₄₀ is: Ser or Gly;
Xaa₄₁ is: Ala, Asp, Arg, Glu, Lys, or Gly;
Xaa₄₂ is: Pro, Ala, Lys, NH₂, or is absent;
Xaa₄₃ is: Pro, Ala, Lys, NH₂, or is absent;
Xaa₄₄ is: Pro, Ala, Arg, Lys, His, NH₂, or is absent;
Xaa₄₅ is: Ser, His, Pro, Lys, Arg, NH₂, or is absent;
Xaa₄₆ is: His, Ser, Arg, Lys, NH₂, or is absent;
Xaa₄₇ is: His, Ser, Arg, Lys, NH₂, or is absent; and
Xaa₄₈ is: Lys, NH₂, or is absent;
Xaa₄₉ is: Pro, His, Lys, NH₂, or is absent;
Xaa₅₀ is: Ser, His, Lys, NH₂, or is absent; and
Xaa₅₁ is: Lys, NH₂, or is absent;
wherein said extended GLP-1 peptide is modified at a single Lys which occurs at one of Xaa₄₂, Xaa₄₃, Xaa₄₄, Xaa₄₅, Xaa₄₆, Xaa₄₇, Xaa₄₈, Xaa₄₉, Xaa₅₀, or Xaa₅₁; and
provided that if Xaa₃₈, Xaa₃₉, Xaa₄₀, Xaa₄₁, Xaa₄₂, Xaa₄₃, Xaa₄₄, Xaa₄₅, Xaa₄₆, Xaa₄₇, Xaa_{48,} Xaa_{49,} or Xaa₅₀, is absent each amino acid downstream is absent.

23. The GLP-1 compound of claim 22, wherein said GLP-1 peptide is modified at a Lys, and said Lys occurs at either Xaa₃₇, Xaa₃₈, Xaa₃₉, Xaa₄₀, Xaa₄₁, Xaa₄₂, Xaa₄₃, Xaa₄₄, Xaa₄₅, Xaa₄₆, Xaa₄₇, Xaa₄₈, Xaa₄₉, Xaa₅₀ or Xaa₅₁.

24-25. (canceled)

26. A GLP-1 compound comprising a GLP-1 peptide modified with a reactive group that reacts with a thiol group on a blood component to form a covalent bond, wherein said reactive group is a succinimidyl group, said GLP-1 peptide having the amino acid sequence of formula 1 (SEQ ID NO:1)
Formula 1

| | |
|---|---|
| Xaa₇-Xaa₈-Glu-Gly-Thr-Xaa₁₂-Thr-Ser- | (SEQ ID NO:1) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Lys-Gly-Arg- | |
| Xaa₃₇ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val, or Ile; and
Xaa₃₇ is: L-Cys, D-Cys, homocysteine, or penicillamine;
wherein said GLP-1 peptide is modified at Xaa₃₇; and
provided that the GLP-1 compound does not have the sequence of GLP-1(7-37)OH, GLP-1(7-36)-NH₂, Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser⁸-GLP-1(7-37)OH, Ser⁸GLP-1(7-36)NH₂, Thr⁸-GLP-1(7-37)OH, Thr⁸-GLP-1(7-36)NH₂, Val⁸-Tyr¹²-GLP-1(7-37)OH, Val⁸-Tyr¹²-GLP-1(7-36)NH₂, Val⁸-Tyr¹⁶-GLP-1(7-37)OH, Val⁸-Tyr¹⁶-GLP-1(7-36)NH₂, Val⁸-Glu²²-GLP-1(7-37)OH, Val⁸-Glu²²-GLP-1(7-36)NH₂, Gly⁸-Glu²²-GLP-1(7-37)OH, Gly⁸-Glu²²-GLP-1(7-36)NH₂, Val⁸-Asp²²-GLP-1(7-37)OH, Val⁸-Asp²²-GLP-1(7-36)NH₂, Gly⁸-Asp²²-GLP-1(7-37)OH, Gly⁸-Asp²²-GLP-1(7-36)NH₂, Val⁸-Lys²²-GLP-1(7-37)OH, Val⁸-Lys²²-GLP-1(7-36)NH₂, Gly⁸-Lys²²-GLP-1(7-37)OH, Gly⁸-Lys²²-GLP-1(7-36)NH₂, Leu⁸-Glu²²-GLP-1(7-37)OH, Leu⁸-Glu²²-GLP-1(7-36)NH₂, Ile⁸-Glu²²-GLP-1(7-37)OH, Ile⁸-Glu²²-GLP-1(7-36)NH₂, Leu⁸-Asp²²-GLP-1(7-37)OH, Leu⁸-Asp²²-GLP-1(7-36)NH₂, Ile⁸-Asp²²-GLP-1(7-37)OH, Ile⁸-Asp²²-GLP-1(7-36)NH₂, Leu⁸-Lys²²-GLP-1(7-37)OH, Leu⁸-Lys²²-GLP-1(7-36)NH₂, Ile⁸-Lys²²-GLP-1(7-37)OH, Ile⁸-Lys²²-GLP-1(7-36)NH₂, Ser⁸-Glu²²-GLP-1(7-37)OH, Ser⁸-Glu²²-GLP-1(7-36)NH₂, Thr⁸-Glu²²-GLP-1(7-37)OH, Thr⁸-Glu²²-GLP-1(7-36)NH₂, Ser⁸-Asp²²-GLP-1(7-37)OH, Ser⁸-Asp²²-GLP-1(7-36)NH₂, Thr⁸-Asp²²-GLP-1(7-37)OH, Thr⁸-Asp²²-GLP-1(7-36)NH₂, Ser⁸-Lys²²-GLP-1(7-37)OH, Ser⁸-Lys²²-GLP-1(7-36)NH₂, Thr⁸-Lys²²-GLP-1(7-37)OH, Thr⁸-Lys²²-GLP-1(7-36)NH₂, Glu²²-GLP-1(7-37)OH, Glu²²-GLP-1(7-36)NH₂, Asp²²-GLP-1(7-37)OH, Asp²²-GLP-1(7-36)NH₂, Lys²²-GLP-1(7-37)OH, Lys²²-GLP-1(7-36)NH₂, Val⁸-Ala²⁷-GLP-1(7-37)OH, Val⁸-Glu²²-Ala²⁷-GLP-1(7-37)OH, Val⁸-Glu³⁰-GLP-1(7-37)OH, Val⁸-Glu³⁰-GLP-1(7-36)NH₂, Gly⁸-Glu³⁰-GLP-1(7-37)OH, Gly⁸-Glu³⁰-GLP-1(7-36)NH₂, Leu⁸-Glu³⁰-GLP-1(7-37)OH, Leu⁸-Glu³⁰-GLP-1(7-36)NH₂, Ile⁸-Glu³⁰-GLP-1(7-37)OH, Ile⁸-Glu³⁰-GLP-1(7-36)NH₂, Ser⁸-Glu³⁰-GLP-1(7-37)OH, Ser⁸-Glu³⁰-GLP-1(7-36)NH₂, Thr⁸-Glu³⁰-GLP-1(7-37)OH, Thr⁸-Glu³⁰-GLP-1(7-36)NH₂, Val⁸-His³⁷-GLP-1(7-37)OH, Val⁸-His³⁷-GLP-1(7-36)NH₂, Gly⁸-His³⁷-GLP-1(7-37)OH, Gly⁸-His³⁷-GLP-1(7-36)NH_{2,} Leu⁸-His³⁷-GLP-1(7-37)OH, Leu⁸-His³⁷-GLP-1(7-36)NH_{2,} Ile⁸-His³⁷-GLP-1(7-37)OH, Ile⁸-His³⁷-GLP-1(7-36)NH_{2,} Ser⁸-His³⁷-GLP-1(7-37)OH, Ser⁸-His³⁷-GLP-1(7-36)NH_{2,} Thr⁸-His³⁷-GLP-1(7-37)OH, Thr⁸-His³⁷-GLP-1(7-36)NH₂.

27. A GLP-1 compound comprising an extended GLP-1 peptide modified with a reactive group that reacts with a thiol group on a blood component to form a covalent bond, wherein said reactive group is a succinimidyl group, said extended GLP-1 peptide having the amino acid sequence of formula 3 (SEQ ID NO:3)
Formula 3

| | |
|---|---|
| Xaa₇-Xaa₈-Glu-Gly-Thr-Xaa₁₂-Thr-Ser- | (SEQ ID NO:3) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Xaa₃₄-Gly- | |
| Xaa₃₆-Xaa₃₇-Xaa₃₈-Xaa₃₉-Xaa₄₀-Xaa₄₁- | |
| Xaa₄₂-Xaa₄₃-Xaa₄₄-Xaa₄₅-Xaa₄₆-Xaa₄₇- | |
| Xaa₄₈ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Tyr, or Trp;
Xaa₂₂ is: Gly, Glu, Asp, or Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val or Ile;
Xaa₃₄ is: Lys, Asp, Arg, or Glu;
Xaa₃₆ is: Gly, Pro, or Arg;
Xaa₃₇ is: Gly, Pro, Ser, L-Cys, D-Cys, homocysteine, or penicillamine;
Xaa₃₈ is: Ser, Pro, His, L-Cys, D-Cys, homocysteine, penicillamine, NH₂;
Xaa₃₉ is: Ser, Arg, Thr, Trp, Lys, L-Cys, D-Cys, homocysteine, penicillamine, NH₂, or is absent;
Xaa₄₀ is: Ser, Gly, L-Cys, D-Cys, homocysteine, penicillamine, NH₂, or is absent;
Xaa₄₁ is: Ala, Asp, Arg, Glu, Lys, Gly, L-Cys, D-Cys, homocysteine, penicillamine, NH₂, or is absent;
Xaa₄₂ is: Pro, Ala, L-Cys, D-Cys, homocysteine, penicillamine, NH₂, or is absent;
Xaa₄₃ is: Pro, Ala, L-Cys, D-Cys, homocysteine, penicillamine, NH₂, or is absent;
Xaa₄₄ is: Pro, Ala, Arg, Lys, His, L-Cys, D-Cys, homocysteine, penicillamine, NH₂, or is absent;
Xaa₄₅ is: Ser, His, Pro, Lys, Arg, L-Cys, D-Cys, homocysteine, penicillamine, NH₂, or is absent;
Xaa₄₆ is: His, Ser, Arg, Lys, L-Cys, D-Cys, homocysteine, penicillamine, NH₂, or is absent;
Xaa₄₇ is: His, Ser, Arg, Lys, L-Cys, D-Cys, homocysteine, penicillamine, NH₂, or is absent; and
Xaa₄₈ is: L-Cys, D-Cys, homocysteine, penicillamine, NH₂, or is absent;
wherein said extended GLP-1 peptide contains a single L-Cys, D-Cys, homocysteine, or penicillamine which occurs at one of Xaa₃₇, Xaa₃₈, Xaa₃₉, Xaa₄₀, Xaa₄₁, Xaa₄₂, Xaa₄₃, Xaa₄₄, Xaa₄₅, Xaa₄₆, Xaa₄₇, or Xaa₄₈, said GLP-1 is modified at said single L-Cys, D-Cys, homocysteine, or penicillamine; and provided that if Xaa₃₉, Xaa₄₀, Xaa₄₁, Xaa₄₂, Xaa₄₃, Xaa₄₄, Xaa₄₅, Xaa₄₆, or Xaa₄₇ is absent each amino acid downstream is absent and further provided that the GLP-1 peptide does not have the following C-terminal amino acid extension beginning at Xaa₃₆: Gly-Pro-Ser-Ser-Gly-Ala-Pro-Pro-Pro-Ser-NH₂.

28. A GLP-1 compound comprising an extended GLP-1 peptide modified with a reactive group that reacts with a thiol group on a blood component to form a covalent bond, wherein said reactive group is a succinimidyl group, said extended GLP-1 peptide having the amino acid sequence of formula 6 (SEQ ID NO:6)
Formula 6

| | |
|---|---|
| Xaa₇-Xaa₈-Glu-Gly-Thr-Xaa₁₂-Thr-Ser- | (SEQ ID NO:6) |
| Asp-Xaa₁₆-Ser-Xaa₁₈-Xaa₁₉-Xaa₂₀-Glu- | |
| Xaa₂₂-Gln-Ala-Xaa₂₅-Lys-Xaa₂₇-Phe- | |
| Ile-Xaa₃₀-Trp-Leu-Xaa₃₃-Xaa₃₄-Gly- | |
| Xaa₃₆-Xaa₃₇-Xaa₃₈-Xaa₃₉-Xaa₄₀-Xaa₄₁- | |
| Xaa₄₂-Xaa₄₃-Xaa₄₄-Xaa₄₅-Xaa₄₆-Xaa₄₇- | |
| Xaa₄₈-Xaa₄₉-Xaa₅₀-Xaa₅₁ | |

wherein:
Xaa₇ is: L-histidine, D-histidine, desamino-histidine, 2-amino-histidine, β-hydroxy-histidine, homohistidine, α-fluoromethyl-histidine, or α-methyl-histidine;
Xaa₈ is: Ala, Gly, Val, Leu, Ile, Ser, or Thr;
Xaa₁₂ is: Phe, Trp, or Tyr;
Xaa₁₆ is: Val, Trp, Ile, Leu, Phe, or Tyr;
Xaa₁₈ is: Ser, Trp, Tyr, Phe, Lys, Ile, Leu, Val;
Xaa₁₉ is: Tyr, Trp, or Phe;
Xaa₂₀ is: Leu, Phe, Tyr, or Trp;
Xaa₂₂ is: Gly, Glu, Asp, or Lys;
Xaa₂₅ is: Ala, Val, Ile, or Leu;
Xaa₂₇ is: Glu, Ile, or Ala;
Xaa₃₀ is: Ala or Glu;
Xaa₃₃ is: Val or Ile;
Xaa₃₄ is: Lys, Asp, Arg, or Glu;
Xaa₃₆ is: Gly, Pro, or Arg;
Xaa₃₇ is: Gly, Pro, Ser;
Xaa₃₈ is: Ser, Pro, or His;
Xaa₃₉ is: Ser, Arg, Thr, Trp, or Lys;
Xaa₄₀ is: Ser or Gly;
Xaa₄₁ is: Ala, Asp, Arg, Glu, Lys, or Gly;
Xaa₄₂ is: Pro, Ala, L-Cys, D-Cys, homocysteine, penicillamine, NH₂, or is absent;
Xaa₄₃ is: Pro, Ala, L-Cys, D-Cys, homocysteine, penicillamine, NH₂, or is absent;
Xaa₄₄ is: Pro, Ala, Arg, Lys, His, NH₂, L-Cys, D-Cys, homocysteine, penicillamine, NH₂, or is absent;
Xaa₄₅ is: Ser, His, Pro, Lys, Arg, Gly, L-Cys, D-Cys, homocysteine, penicillamine, NH₂, or is absent;
Xaa₄₆ is: His, Ser, Arg, Lys, Pro, Gly, L-Cys, D-Cys, homocysteine, penicillamine, NH₂, or is absent;
Xaa₄₇ is: His, Ser, Arg, Lys, L-Cys, D-Cys, homocysteine, penicillamine, NH₂, or is absent;
Xaa₄₈ is: Gly, His, L-Cys, D-Cys, homocysteine, penicillamine, NH₂, or is absent;
Xaa₄₉ is: Pro, His, L-Cys, D-Cys, homocysteine, penicillamine, NH₂, or is absent;
Xaa₅₀ is: Ser, His, Ser-NH₂, His-NH₂, L-Cys, D-Cys, homocysteine, penicillamine, NH₂, or is absent; and
Xaa₅₁ is: L-Cys, D-Cys, homocysteine, penicillamine, NH₂, or is absent;
wherein said extended GLP-1 peptide contains a single L-Cys, D-Cys, homocysteine, or penicillamine which occurs at one of Xaa₄₂, Xaa₄₃, Xaa₄₄, Xaa₄₅, Xaa₄₆, Xaa₄₇, Xaa₄₈, Xaa_{49,} Xaa_{50,} or Xaa₅₁ said GLP-1 is modified at said single L-Cys, D-Cys, homocysteine, or penicillamine; and provided that if Xaa₄₂, Xaa₄₃, Xaa₄₄, Xaa₄₅, Xaa₄₆, Xaa_{47,} Xaa_{48,} Xaa_{49,} or Xaa₅₀, is absent each amino acid downstream is absent and further provided that if Xaa₃₆ is Arg and Xaa₃₇ is Gly or Ser, the GLP-1 peptide does not have the following C-terminal amino acid extension beginning at Xaa₃₈: Ser-Ser-Gly-Ala-Pro-Pro-Pro-Ser-NH₂.

29. The GLP-1 compound of claim 2 provided that the GLP-1 compound does not differ from GLP-1(7-37)OH or GLP-1(7-36)NH₂ by more than 5 amino acids.

30. The GLP-1 compound of claim 29 provided that the GLP-1 compound does not differ from GLP-1(7-37)OH or GLP-1(7-36)NH₂ by more than 4 amino acids.

31. The GLP-1 compound of claim 30 provided that the GLP-1 compound does not differ from GLP-1(7-37)OH or GLP-1(7-36)NH₂ by more than 3 amino acids.

32. The GLP-1 compound of claim 7 wherein the first 31 amino acids of the peptide do not differ from GLP-1(7-37) by more than 6 amino acids.

33. The GLP-1 compound of claim 32 wherein the first 31 amino acids of the peptide do not differ from GLP-1(7-37) by more than 5 amino acids.

34. The GLP-1 compound of claim 33 wherein the first 31 amino acids of the peptide do not differ from GLP-1(7-37) by more than 4 amino acids.

35. The GLP-1 compound of claim 34 wherein the first 31 amino acids of the peptide do not differ from GLP-1(7-37) by more than 3 amino acids.

36. A conjugate comprising a GLP-1 compound of claim 1 covalently bonded ex vivo to a blood component.

37. A conjugate comprising a GLP-1 compound of claim 1 covalently bonded ex vivo to a blood serum albumin.

38. A method for extending the in vivo half-life of a GLP-1 compound as claimed in claim 1, comprising reacting said reactive group of said pharmaceutical composition with a thiol group on a blood component in vivo.

39. A method for extending the in vivo half-life of a GLP-1 compound as claimed in claim 1, comprising reacting said reactive group of said pharmaceutical composition with a thiol group on blood serum albumin in vivo.

40. A method of stimulating the GLP-1 receptor in a subject in need of such stimulation, said method comprising the step of administering to the subject an effective amount of the GLP-1 compound of claim 1.

41. The method of claim 40 wherein the subject is being treated for non-insulin dependent diabetes.

42. The method of claim 40 wherein the subject is being treated prophylactically for non insulin dependent diabetes.

43. The method of claim 40 wherein the subject is being treated for obesity.

44. The method of claim 40 wherein the subject is being treated for stroke, myocardial infarction, stroke, stress-induced hyperglycemia, or irritable bowel syndrome.

45-47. (canceled)


SEQUENCE LISTING (24 sequences)

SEQ ID NO: 1 - protein, 31 aa, Artificial
  Synthetic constructs
XXEGTXTSDXSXXXEXQAXKXFIXWLXKGRX

SEQ ID NO: 2 - protein, 31 aa, Artificial
  Synthetic construct
XXEGTFTSDXSXYLEXQAXKEFIAWLXKGRX

SEQ ID NO: 3 - protein, 42 aa, Artificial
  Synthetic construct
XXEGTXTSDXSXXXEXQAXKXFIXWLXXGXXXXXXXXXXXXX

SEQ ID NO: 4 - protein, 42 aa, Artificial
  Synthetic construct
XXEGTFTSDXSSYKEXQAXKEFIAWLXXGXXXXXXXXXXXXX

SEQ ID NO: 5 - protein, 42 aa, Artificial
  Synthetic construct
XXEGTFTSDVSSYKEXQAXKEFIAWLXKGGPXXXXXXXXXXX

SEQ ID NO: 6 - protein, 45 aa, Artificial
  Synthetic construct
XXEGTXTSDXSXXXEXQAXKXFIXWLXXGXXXXXXXXXXXXXXXX

SEQ ID NO: 7 - protein, 45 aa, Artificial
  Synthetic construct
HAEGTFTSDVSSYLEGQAAKEFIAWLVKGGPXXXXXXXXXXXXXX

SEQ ID NO: 8 - protein, 31 aa, Artificial
  Synthetic construct
XXEGTXTSDXSXXXEXQAXKXFIXWLXKGRK

SEQ ID NO: 9 - protein, 31 aa, Artificial
  Synthetic construct
XXEGTFTSDXSXYLEXQAXKEFIAWLXKGRK

SEQ ID NO: 10 - protein, 42 aa, Artificial; identical to SEQ ID NO: 3
  Synthetic construct

SEQ ID NO: 11 - protein, 42 aa, Artificial; identical to SEQ ID NO: 4
  Synthetic construct

SEQ ID NO: 12 - protein, 42 aa, Artificial; identical to SEQ ID NO: 5
  Synthetic construct

SEQ ID NO: 13 - protein, 45 aa, Artificial; identical to SEQ ID NO: 6
  Synthetic construct

SEQ ID NO: 14 - protein, 45 aa, Artificial; identical to SEQ ID NO: 7
  Synthetic construct

SEQ ID NO: 15 - protein, 31 aa, Artificial; identical to SEQ ID NO: 1
  Synthetic construct

SEQ ID NO: 16 - protein, 31 aa, Artificial
  Synthetic construct
HAEGTFTSDVSSYLEGQAAKEFIAWLVKGRG

SEQ ID NO: 17 - protein, 39 aa, Artificial
  Synthetic construct
HVEGTFTSDVSSYLEEQAAKEFIAWLIKGGPSSGAPPPC

SEQ ID NO: 18 - protein, 39 aa, Artificial; identical to SEQ ID NO: 17
  Synthetic construct

SEQ ID NO: 19 - protein, 32 aa, Artificial
  Synthetic construct
HVEGTFTSDVSSYLEEQAAKEFIAWLIKGRGC

SEQ ID NO: 20 - protein, 32 aa, Artificial; identical to SEQ ID NO: 19
  Synthetic construct

SEQ ID NO: 21 - protein, 32 aa, Artificial
  Synthetic construct
HVEGTFTSDVSSYLEEQAAKEFIAWLIKGRGK

SEQ ID NO: 22 - protein, 32 aa, Artificial; identical to SEQ ID NO: 21
  Synthetic construct

SEQ ID NO: 23 - protein, 39 aa, Heloderma suspectum
  MISC_FEATURE (1)..(39): Exendin-3
HSDGTFTSDLSKQMEEEAVRLFIEWLKNGGPSSGAPPPS

SEQ ID NO: 24 - protein, 39 aa, Heloderma suspectum
  MISC_FEATURE (1)..(39): Exendin-4
HGEGTFTSDLSKQMEEEAVRLFIEWLKNGGPSSGAPPPS